SEQUENCE LISTING (854 sequences)

SEQ ID NO: 1 - DNA, 1543 bp, Homo sapiens
ggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttatgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatg

SEQ ID NO: 2 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 3 - DNA, 21 bp, Artificial Sequence
  Description of Artificial Sequence Synthetic oligonucleotide
guggcuucaaucuacgaugtt

SEQ ID NO: 4 - DNA, 21 bp, Artificial Sequence
  Description of Artificial Sequence Synthetic oligonucleotide
caucguagauugaagccactt

SEQ ID NO: 5 - DNA, 21 bp, Artificial Sequence
  Description of Artificial Sequence Synthetic oligonucleotide
uuuuuacaguguaucucgatt

SEQ ID NO: 6 - DNA, 21 bp, Artificial Sequence
  Description of Artificial Sequence Synthetic oligonucleotide
ucgagauacacuguaaaaatt

SEQ ID NO: 7 - DNA, 21 bp, Artificial Sequence
  Description of Artificial Sequence Synthetic oligonucleotide
guaucaagacuacgaaccutt

SEQ ID NO: 8 - DNA, 21 bp, Artificial Sequence
  Description of Artificial Sequence Synthetic oligonucleotide
agguucguagucuugauactt

SEQ ID NO: 9 - DNA, 21 bp, Artificial Sequence
  Description of Artificial Sequence Synthetic oligonucleotide
uuuuacaguguaucucgaatt

SEQ ID NO: 10 - DNA, 21 bp, Artificial Sequence
  Description of Artificial Sequence Synthetic oligonucleotide
uucgagauacacuguaaaatt

SEQ ID NO: 11 - DNA, 21 bp, Artificial Sequence
  Description of Artificial Sequence Synthetic oligonucleotide
acauuaacccuacacucggtt

SEQ ID NO: 12 - DNA, 21 bp, Artificial Sequence
  Description of Artificial Sequence Synthetic oligonucleotide
ccgaguguaggguuaaugutt

SEQ ID NO: 13 - DNA, 21 bp, Artificial Sequence
  Description of Artificial Sequence Synthetic oligonucleotide
uguggcuucaaucuacgautt

SEQ ID NO: 14 - DNA, 21 bp, Artificial Sequence
  Description of Artificial Sequence Synthetic oligonucleotide
aucguagauugaagccacatt

SEQ ID NO: 15 - DNA, 21 bp, Artificial Sequence
  Description of Artificial Sequence Synthetic oligonucleotide
cucagcauuucggugcuuctt

SEQ ID NO: 16 - DNA, 21 bp, Artificial Sequence
  Description of Artificial Sequence Synthetic oligonucleotide
gaagcaccgaaaugcugagtt

SEQ ID NO: 17 - DNA, 21 bp, Artificial Sequence
  Description of Artificial Sequence Synthetic oligonucleotide
uuaacccuacacucggaautt

SEQ ID NO: 18 - DNA, 21 bp, Artificial Sequence
  Description of Artificial Sequence Synthetic oligonucleotide
auuccgaguguaggguuaatt

SEQ ID NO: 19 - DNA, 21 bp, Artificial Sequence
  Description of Artificial Sequence Synthetic oligonucleotide
uuacaguguaucucgaagutt

SEQ ID NO: 20 - DNA, 21 bp, Artificial Sequence
  Description of Artificial Sequence Synthetic oligonucleotide
acuucgagauacacuguaatt

SEQ ID NO: 21 - DNA, 21 bp, Artificial Sequence
  Description of Artificial Sequence Synthetic oligonucleotide
acaguguaucucgaagucutt

SEQ ID NO: 22 - DNA, 21 bp, Artificial Sequence
  Description of Artificial Sequence Synthetic oligonucleotide
agacuucgagauacacugutt

SEQ ID NO: 23 - DNA, 21 bp, Artificial Sequence
  Description of Artificial Sequence Synthetic oligonucleotide
gcuucaaucuacgauguuatt

SEQ ID NO: 24 - DNA, 21 bp, Artificial Sequence
  Description of Artificial Sequence Synthetic oligonucleotide
uaacaucguagauugaagctt

SEQ ID NO: 25 - DNA, 21 bp, Artificial Sequence
  Description of Artificial Sequence Synthetic oligonucleotide
auuaacccuacacucggaatt

SEQ ID NO: 26 - DNA, 21 bp, Artificial Sequence
  Description of Artificial Sequence Synthetic oligonucleotide
uuccgaguguaggguuaautt

SEQ ID NO: 27 - DNA, 21 bp, Artificial Sequence
  Description of Artificial Sequence Synthetic oligonucleotide
uuaaugauacugucuaagatt

SEQ ID NO: 28 - DNA, 21 bp, Artificial Sequence
  Description of Artificial Sequence Synthetic oligonucleotide
ucuuagacaguaucauuaatt

SEQ ID NO: 29 - DNA, 21 bp, Artificial Sequence
  Description of Artificial Sequence Synthetic oligonucleotide
aucaagacuacgaaccugatt

SEQ ID NO: 30 - DNA, 21 bp, Artificial Sequence
  Description of Artificial Sequence Synthetic oligonucleotide
ucagguucguagucuugautt

SEQ ID NO: 31 - DNA, 21 bp, Artificial Sequence
  Description of Artificial Sequence Synthetic oligonucleotide
agugaauacaugguagcagtt

SEQ ID NO: 32 - DNA, 21 bp, Artificial Sequence
  Description of Artificial Sequence Synthetic oligonucleotide
cugcuaccauguauucacutt

SEQ ID NO: 33 - DNA, 21 bp, Artificial Sequence
  Description of Artificial Sequence Synthetic oligonucleotide
aauacaugguagcaggguctt

SEQ ID NO: 34 - DNA, 21 bp, Artificial Sequence
  Description of Artificial Sequence Synthetic oligonucleotide
gacccugcuaccauguauutt

SEQ ID NO: 35 - DNA, 21 bp, Artificial Sequence
  Description of Artificial Sequence Synthetic oligonucleotide
cuaagugacuaccacuuautt

SEQ ID NO: 36 - DNA, 21 bp, Artificial Sequence
  Description of Artificial Sequence Synthetic oligonucleotide
auaagugguagucacuuagtt

SEQ ID NO: 37 - DNA, 21 bp, Artificial Sequence
  Description of Artificial Sequence Synthetic oligonucleotide
agcaugaaacuaugcaccutt

SEQ ID NO: 38 - DNA, 21 bp, Artificial Sequence
  Description of Artificial Sequence Synthetic oligonucleotide
aggugcauaguuucaugcutt

SEQ ID NO: 39 - DNA, 21 bp, Artificial Sequence
  Description of Artificial Sequence Synthetic oligonucleotide
uuguggcuucaaucuacgatt

SEQ ID NO: 40 - DNA, 21 bp, Artificial Sequence
  Description of Artificial Sequence Synthetic oligonucleotide
ucguagauugaagccacaatt

SEQ ID NO: 41 - DNA, 21 bp, Artificial Sequence
  Description of Artificial Sequence Synthetic oligonucleotide
ggcuucaaucuacgauguutt

SEQ ID NO: 42 - DNA, 21 bp, Artificial Sequence
  Description of Artificial Sequence Synthetic oligonucleotide
aacaucguagauugaagcctt

SEQ ID NO: 43 - DNA, 21 bp, Artificial Sequence
  Description of Artificial Sequence Synthetic oligonucleotide
auaaugacguauugugaaatt

SEQ ID NO: 44 - DNA, 21 bp, Artificial Sequence
  Description of Artificial Sequence Synthetic oligonucleotide
uuucacaauacgucauuautt

SEQ ID NO: 45 - DNA, 21 bp, Artificial Sequence
  Description of Artificial Sequence Synthetic oligonucleotide
agugauuugcuaucauauatt

SEQ ID NO: 46 - DNA, 21 bp, Artificial Sequence
  Description of Artificial Sequence Synthetic oligonucleotide
uauaugauagcaaaucacutt

SEQ ID NO: 47 - DNA, 21 bp, Artificial Sequence
  Description of Artificial Sequence Synthetic oligonucleotide
caaucuacgauguuaaaactt

SEQ ID NO: 48 - DNA, 21 bp, Artificial Sequence
  Description of Artificial Sequence Synthetic oligonucleotide
guuuuaacaucguagauugtt

SEQ ID NO: 49 - DNA, 21 bp, Artificial Sequence
  Description of Artificial Sequence Synthetic oligonucleotide
acguauugugaaauuuguutt

SEQ ID NO: 50 - DNA, 21 bp, Artificial Sequence
  Description of Artificial Sequence Synthetic oligonucleotide
aacaaauuucacaauacgutt

SEQ ID NO: 51 - DNA, 21 bp, Artificial Sequence
  Description of Artificial Sequence Synthetic oligonucleotide
gacguauugugaaauuugutt

SEQ ID NO: 52 - DNA, 21 bp, Artificial Sequence
  Description of Artificial Sequence Synthetic oligonucleotide
acaaauuucacaauacguctt

SEQ ID NO: 53 - DNA, 21 bp, Artificial Sequence
  Description of Artificial Sequence Synthetic oligonucleotide
acgggugugacagcaguagtt

SEQ ID NO: 54 - DNA, 21 bp, Artificial Sequence
  Description of Artificial Sequence Synthetic oligonucleotide
cuacugcugucacacccgutt

SEQ ID NO: 55 - DNA, 21 bp, Artificial Sequence
  Description of Artificial Sequence Synthetic oligonucleotide
gguaucaagacuacgaacctt

SEQ ID NO: 56 - DNA, 21 bp, Artificial Sequence
  Description of Artificial Sequence Synthetic oligonucleotide
gguucguagucuugauacctt

SEQ ID NO: 57 - DNA, 21 bp, Artificial Sequence
  Description of Artificial Sequence Synthetic oligonucleotide
ugucuaagaauaaugacgutt

SEQ ID NO: 58 - DNA, 21 bp, Artificial Sequence
  Description of Artificial Sequence Synthetic oligonucleotide
acgucauuauucuuagacatt

SEQ ID NO: 59 - DNA, 21 bp, Artificial Sequence
  Description of Artificial Sequence Synthetic oligonucleotide
aacccuacacucggaauuctt

SEQ ID NO: 60 - DNA, 21 bp, Artificial Sequence
  Description of Artificial Sequence Synthetic oligonucleotide
gaauuccgaguguaggguutt

SEQ ID NO: 61 - DNA, 21 bp, Artificial Sequence
  Description of Artificial Sequence Synthetic oligonucleotide
aaaagaggguguucucuautt

SEQ ID NO: 62 - DNA, 21 bp, Artificial Sequence
  Description of Artificial Sequence Synthetic oligonucleotide
auagagaacacccucuuuutt

SEQ ID NO: 63 - DNA, 21 bp, Artificial Sequence
  Description of Artificial Sequence Synthetic oligonucleotide
ggguguucucuauguaggctt

SEQ ID NO: 64 - DNA, 21 bp, Artificial Sequence
  Description of Artificial Sequence Synthetic oligonucleotide
gccuacauagagaacaccctt

SEQ ID NO: 65 - DNA, 21 bp, Artificial Sequence
  Description of Artificial Sequence Synthetic oligonucleotide
ucucuauguaggcuccaaatt

SEQ ID NO: 66 - DNA, 21 bp, Artificial Sequence
  Description of Artificial Sequence Synthetic oligonucleotide
uuuggagccuacauagagatt

SEQ ID NO: 67 - DNA, 21 bp, Artificial Sequence
  Description of Artificial Sequence Synthetic oligonucleotide
gaaggguaucaagacuacgtt

SEQ ID NO: 68 - DNA, 21 bp, Artificial Sequence
  Description of Artificial Sequence Synthetic oligonucleotide
cguagucuugauacccuuctt

SEQ ID NO: 69 - DNA, 21 bp, Artificial Sequence
  Description of Artificial Sequence Synthetic oligonucleotide
aaggguaucaagacuacgatt

SEQ ID NO: 70 - DNA, 21 bp, Artificial Sequence
  Description of Artificial Sequence Synthetic oligonucleotide
ucguagucuugauacccuutt

SEQ ID NO: 71 - DNA, 21 bp, Artificial Sequence
  Description of Artificial Sequence Synthetic oligonucleotide
ggguaucaagacuacgaactt

SEQ ID NO: 72 - DNA, 21 bp, Artificial Sequence
  Description of Artificial Sequence Synthetic oligonucleotide
guucguagucuugauaccctt

SEQ ID NO: 73 - DNA, 21 bp, Artificial Sequence
  Description of Artificial Sequence Synthetic oligonucleotide
gaagccuaagaaauaucuutt

SEQ ID NO: 74 - DNA, 21 bp, Artificial Sequence
  Description of Artificial Sequence Synthetic oligonucleotide
aagauauuucuuaggcuuctt

SEQ ID NO: 75 - DNA, 21 bp, Artificial Sequence
  Description of Artificial Sequence Synthetic oligonucleotide
uuuacaguguaucucgaagtt

SEQ ID NO: 76 - DNA, 21 bp, Artificial Sequence
  Description of Artificial Sequence Synthetic oligonucleotide
cuucgagauacacuguaaatt

SEQ ID NO: 77 - DNA, 21 bp, Artificial Sequence
  Description of Artificial Sequence Synthetic oligonucleotide
uacaguguaucucgaaguctt

SEQ ID NO: 78 - DNA, 21 bp, Artificial Sequence
  Description of Artificial Sequence Synthetic oligonucleotide
gacuucgagauacacuguatt

SEQ ID NO: 79 - DNA, 21 bp, Artificial Sequence
  Description of Artificial Sequence Synthetic oligonucleotide
cacucagcauuucggugcutt

SEQ ID NO: 80 - DNA, 21 bp, Artificial Sequence
  Description of Artificial Sequence Synthetic oligonucleotide
agcaccgaaaugcugagugtt

SEQ ID NO: 81 - DNA, 21 bp, Artificial Sequence
  Description of Artificial Sequence Synthetic oligonucleotide
uggcuucaaucuacgaugutt

SEQ ID NO: 82 - DNA, 21 bp, Artificial Sequence
  Description of Artificial Sequence Synthetic oligonucleotide
acaucguagauugaagccatt

SEQ ID NO: 83 - DNA, 21 bp, Artificial Sequence
  Description of Artificial Sequence Synthetic oligonucleotide
cuucaaucuacgauguuaatt

SEQ ID NO: 84 - DNA, 21 bp, Artificial Sequence
  Description of Artificial Sequence Synthetic oligonucleotide
uuaacaucguagauugaagtt

SEQ ID NO: 85 - DNA, 21 bp, Artificial Sequence
  Description of Artificial Sequence Synthetic oligonucleotide
aaucuacgauguuaaaacatt

SEQ ID NO: 86 - DNA, 21 bp, Artificial Sequence
  Description of Artificial Sequence Synthetic oligonucleotide
uguuuuaacaucguagauutt

SEQ ID NO: 87 - DNA, 21 bp, Artificial Sequence
  Description of Artificial Sequence Synthetic oligonucleotide
aacaccuaagugacuaccatt

SEQ ID NO: 88 - DNA, 21 bp, Artificial Sequence
  Description of Artificial Sequence Synthetic oligonucleotide
ugguagucacuuagguguutt

SEQ ID NO: 89 - DNA, 21 bp, Artificial Sequence
  Description of Artificial Sequence Synthetic oligonucleotide
uaagugacuaccacuuauutt

SEQ ID NO: 90 - DNA, 21 bp, Artificial Sequence
  Description of Artificial Sequence Synthetic oligonucleotide
aauaagugguagucacuuatt

SEQ ID NO: 91 - DNA, 21 bp, Artificial Sequence
  Description of Artificial Sequence Synthetic oligonucleotide
uguucagaaguuguuagugtt

SEQ ID NO: 92 - DNA, 21 bp, Artificial Sequence
  Description of Artificial Sequence Synthetic oligonucleotide
cacuaacaacuucugaacatt

SEQ ID NO: 93 - DNA, 21 bp, Artificial Sequence
  Description of Artificial Sequence Synthetic oligonucleotide
aaguuguuagugauuugcutt

SEQ ID NO: 94 - DNA, 21 bp, Artificial Sequence
  Description of Artificial Sequence Synthetic oligonucleotide
agcaaaucacuaacaacuutt

SEQ ID NO: 95 - DNA, 21 bp, Artificial Sequence
  Description of Artificial Sequence Synthetic oligonucleotide
auaagauuuuuaggugucutt

SEQ ID NO: 96 - DNA, 21 bp, Artificial Sequence
  Description of Artificial Sequence Synthetic oligonucleotide
agacaccuaaaaaucuuautt

SEQ ID NO: 97 - DNA, 21 bp, Artificial Sequence
  Description of Artificial Sequence Synthetic oligonucleotide
aaugacguauugugaaauutt

SEQ ID NO: 98 - DNA, 21 bp, Artificial Sequence
  Description of Artificial Sequence Synthetic oligonucleotide
aauuucacaauacgucauutt

SEQ ID NO: 99 - DNA, 21 bp, Artificial Sequence
  Description of Artificial Sequence Synthetic oligonucleotide
gcaugaaacuaugcaccuatt

SEQ ID NO: 100 - DNA, 21 bp, Artificial Sequence
  Description of Artificial Sequence Synthetic oligonucleotide
uaggugcauaguuucaugctt

SEQ ID NO: 101 - DNA, 21 bp, Artificial Sequence
  Description of Artificial Sequence Synthetic oligonucleotide
uaugcaccuauaaauacuatt

SEQ ID NO: 102 - DNA, 21 bp, Artificial Sequence
  Description of Artificial Sequence Synthetic oligonucleotide
uaguauuuauaggugcauatt

SEQ ID NO: 103 - DNA, 21 bp, Artificial Sequence
  Description of Artificial Sequence Synthetic oligonucleotide
uaccauuuugcgauguguutt

SEQ ID NO: 104 - DNA, 21 bp, Artificial Sequence
  Description of Artificial Sequence Synthetic oligonucleotide
aacacaucgcaaaaugguatt

SEQ ID NO: 105 - DNA, 21 bp, Artificial Sequence
  Description of Artificial Sequence Synthetic oligonucleotide
auuuuuaucccaucucacutt

SEQ ID NO: 106 - DNA, 21 bp, Artificial Sequence
  Description of Artificial Sequence Synthetic oligonucleotide
agugagaugggauaaaaautt

SEQ ID NO: 107 - DNA, 21 bp, Artificial Sequence
  Description of Artificial Sequence Synthetic oligonucleotide
uuaucccaucucacuuuaatt

SEQ ID NO: 108 - DNA, 21 bp, Artificial Sequence
  Description of Artificial Sequence Synthetic oligonucleotide
uuaaagugagaugggauaatt

SEQ ID NO: 109 - DNA, 21 bp, Artificial Sequence
  Description of Artificial Sequence Synthetic oligonucleotide
uaacccuacacucggaauutt

SEQ ID NO: 110 - DNA, 21 bp, Artificial Sequence
  Description of Artificial Sequence Synthetic oligonucleotide
aauuccgaguguaggguuatt

SEQ ID NO: 111 - DNA, 21 bp, Artificial Sequence
  Description of Artificial Sequence Synthetic oligonucleotide
aggguguucucuauguaggtt

SEQ ID NO: 112 - DNA, 21 bp, Artificial Sequence
  Description of Artificial Sequence Synthetic oligonucleotide
ccuacauagagaacacccutt

SEQ ID NO: 113 - DNA, 21 bp, Artificial Sequence
  Description of Artificial Sequence Synthetic oligonucleotide
cucuauguaggcuccaaaatt

SEQ ID NO: 114 - DNA, 21 bp, Artificial Sequence
  Description of Artificial Sequence Synthetic oligonucleotide
uuuuggagccuacauagagtt

SEQ ID NO: 115 - DNA, 21 bp, Artificial Sequence
  Description of Artificial Sequence Synthetic oligonucleotide
aaccaaacaggguguggcatt

SEQ ID NO: 116 - DNA, 21 bp, Artificial Sequence
  Description of Artificial Sequence Synthetic oligonucleotide
ugccacacccuguuugguutt

SEQ ID NO: 117 - DNA, 21 bp, Artificial Sequence
  Description of Artificial Sequence Synthetic oligonucleotide
gaggguguucucuauguagtt

SEQ ID NO: 118 - DNA, 21 bp, Artificial Sequence
  Description of Artificial Sequence Synthetic oligonucleotide
cuacauagagaacacccuctt

SEQ ID NO: 119 - DNA, 21 bp, Artificial Sequence
  Description of Artificial Sequence Synthetic oligonucleotide
uucucuauguaggcuccaatt

SEQ ID NO: 120 - DNA, 21 bp, Artificial Sequence
  Description of Artificial Sequence Synthetic oligonucleotide
uuggagccuacauagagaatt

SEQ ID NO: 121 - DNA, 21 bp, Artificial Sequence
  Description of Artificial Sequence Synthetic oligonucleotide
gugacaaauguuggaggagtt

SEQ ID NO: 122 - DNA, 21 bp, Artificial Sequence
  Description of Artificial Sequence Synthetic oligonucleotide
cuccuccaacauuugucactt

SEQ ID NO: 123 - DNA, 21 bp, Artificial Sequence
  Description of Artificial Sequence Synthetic oligonucleotide
gucaaaaaggaccaguuggtt

SEQ ID NO: 124 - DNA, 21 bp, Artificial Sequence
  Description of Artificial Sequence Synthetic oligonucleotide
ccaacugguccuuuuugactt

SEQ ID NO: 125 - DNA, 21 bp, Artificial Sequence
  Description of Artificial Sequence Synthetic oligonucleotide
ucaagacuacgaaccugaatt

SEQ ID NO: 126 - DNA, 21 bp, Artificial Sequence
  Description of Artificial Sequence Synthetic oligonucleotide
uucagguucguagucuugatt

SEQ ID NO: 127 - DNA, 21 bp, Artificial Sequence
  Description of Artificial Sequence Synthetic oligonucleotide
cagcauuucggugcuuccctt

SEQ ID NO: 128 - DNA, 21 bp, Artificial Sequence
  Description of Artificial Sequence Synthetic oligonucleotide
gggaagcaccgaaaugcugtt

SEQ ID NO: 129 - DNA, 21 bp, Artificial Sequence
  Description of Artificial Sequence Synthetic oligonucleotide
guuguucagaaguuguuagtt

SEQ ID NO: 130 - DNA, 21 bp, Artificial Sequence
  Description of Artificial Sequence Synthetic oligonucleotide
cuaacaacuucugaacaactt

SEQ ID NO: 131 - DNA, 21 bp, Artificial Sequence
  Description of Artificial Sequence Synthetic oligonucleotide
gaaguuguuagugauuugctt

SEQ ID NO: 132 - DNA, 21 bp, Artificial Sequence
  Description of Artificial Sequence Synthetic oligonucleotide
gcaaaucacuaacaacuuctt

SEQ ID NO: 133 - DNA, 21 bp, Artificial Sequence
  Description of Artificial Sequence Synthetic oligonucleotide
aguuguuagugauuugcuatt

SEQ ID NO: 134 - DNA, 21 bp, Artificial Sequence
  Description of Artificial Sequence Synthetic oligonucleotide
uagcaaaucacuaacaacutt

SEQ ID NO: 135 - DNA, 21 bp, Artificial Sequence
  Description of Artificial Sequence Synthetic oligonucleotide
uuagugauuugcuaucauatt

SEQ ID NO: 136 - DNA, 21 bp, Artificial Sequence
  Description of Artificial Sequence Synthetic oligonucleotide
uaugauagcaaaucacuaatt

SEQ ID NO: 137 - DNA, 21 bp, Artificial Sequence
  Description of Artificial Sequence Synthetic oligonucleotide
gugauuugcuaucauauautt

SEQ ID NO: 138 - DNA, 21 bp, Artificial Sequence
  Description of Artificial Sequence Synthetic oligonucleotide
auauaugauagcaaaucactt

SEQ ID NO: 139 - DNA, 21 bp, Artificial Sequence
  Description of Artificial Sequence Synthetic oligonucleotide
uaaugacguauugugaaautt

SEQ ID NO: 140 - DNA, 21 bp, Artificial Sequence
  Description of Artificial Sequence Synthetic oligonucleotide
auuucacaauacgucauuatt

SEQ ID NO: 141 - DNA, 21 bp, Artificial Sequence
  Description of Artificial Sequence Synthetic oligonucleotide
cuaugcaccuauaaauacutt

SEQ ID NO: 142 - DNA, 21 bp, Artificial Sequence
  Description of Artificial Sequence Synthetic oligonucleotide
aguauuuauaggugcauagtt

SEQ ID NO: 143 - DNA, 21 bp, Artificial Sequence
  Description of Artificial Sequence Synthetic oligonucleotide
auuagccauggauguauuctt

SEQ ID NO: 144 - DNA, 21 bp, Artificial Sequence
  Description of Artificial Sequence Synthetic oligonucleotide
gaauacauccauggcuaautt

SEQ ID NO: 145 - DNA, 21 bp, Artificial Sequence
  Description of Artificial Sequence Synthetic oligonucleotide
uuagccauggauguauucatt

SEQ ID NO: 146 - DNA, 21 bp, Artificial Sequence
  Description of Artificial Sequence Synthetic oligonucleotide
ugaauacauccauggcuaatt

SEQ ID NO: 147 - DNA, 21 bp, Artificial Sequence
  Description of Artificial Sequence Synthetic oligonucleotide
gaaaggacuuucaaaggcctt

SEQ ID NO: 148 - DNA, 21 bp, Artificial Sequence
  Description of Artificial Sequence Synthetic oligonucleotide
ggccuuugaaaguccuuuctt

SEQ ID NO: 149 - DNA, 21 bp, Artificial Sequence
  Description of Artificial Sequence Synthetic oligonucleotide
caaaagaggguguucucuatt

SEQ ID NO: 150 - DNA, 21 bp, Artificial Sequence
  Description of Artificial Sequence Synthetic oligonucleotide
uagagaacacccucuuuugtt

SEQ ID NO: 151 - DNA, 21 bp, Artificial Sequence
  Description of Artificial Sequence Synthetic oligonucleotide
agaggguguucucuauguatt

SEQ ID NO: 152 - DNA, 21 bp, Artificial Sequence
  Description of Artificial Sequence Synthetic oligonucleotide
uacauagagaacacccucutt

SEQ ID NO: 153 - DNA, 21 bp, Artificial Sequence
  Description of Artificial Sequence Synthetic oligonucleotide
gguguucucuauguaggcutt

SEQ ID NO: 154 - DNA, 21 bp, Artificial Sequence
  Description of Artificial Sequence Synthetic oligonucleotide
agccuacauagagaacacctt

SEQ ID NO: 155 - DNA, 21 bp, Artificial Sequence
  Description of Artificial Sequence Synthetic oligonucleotide
guguucucuauguaggcuctt

SEQ ID NO: 156 - DNA, 21 bp, Artificial Sequence
  Description of Artificial Sequence Synthetic oligonucleotide
gagccuacauagagaacactt

SEQ ID NO: 157 - DNA, 21 bp, Artificial Sequence
  Description of Artificial Sequence Synthetic oligonucleotide
uguucucuauguaggcucctt

SEQ ID NO: 158 - DNA, 21 bp, Artificial Sequence
  Description of Artificial Sequence Synthetic oligonucleotide
ggagccuacauagagaacatt

SEQ ID NO: 159 - DNA, 21 bp, Artificial Sequence
  Description of Artificial Sequence Synthetic oligonucleotide
ggagcaguggugacgggugtt

SEQ ID NO: 160 - DNA, 21 bp, Artificial Sequence
  Description of Artificial Sequence Synthetic oligonucleotide
cacccgucaccacugcucctt

SEQ ID NO: 161 - DNA, 21 bp, Artificial Sequence
  Description of Artificial Sequence Synthetic oligonucleotide
caguggugacgggugugactt

SEQ ID NO: 162 - DNA, 21 bp, Artificial Sequence
  Description of Artificial Sequence Synthetic oligonucleotide
gucacacccgucaccacugtt

SEQ ID NO: 163 - DNA, 21 bp, Artificial Sequence
  Description of Artificial Sequence Synthetic oligonucleotide
uaucaagacuacgaaccugtt

SEQ ID NO: 164 - DNA, 21 bp, Artificial Sequence
  Description of Artificial Sequence Synthetic oligonucleotide
cagguucguagucuugauatt

SEQ ID NO: 165 - DNA, 21 bp, Artificial Sequence
  Description of Artificial Sequence Synthetic oligonucleotide
cucccaguuucuugagauctt

SEQ ID NO: 166 - DNA, 21 bp, Artificial Sequence
  Description of Artificial Sequence Synthetic oligonucleotide
gaucucaagaaacugggagtt

SEQ ID NO: 167 - DNA, 21 bp, Artificial Sequence
  Description of Artificial Sequence Synthetic oligonucleotide
gacagauguuccauccugutt

SEQ ID NO: 168 - DNA, 21 bp, Artificial Sequence
  Description of Artificial Sequence Synthetic oligonucleotide
acaggauggaacaucuguctt

SEQ ID NO: 169 - DNA, 21 bp, Artificial Sequence
  Description of Artificial Sequence Synthetic oligonucleotide
aaugugcccagucaugacatt

SEQ ID NO: 170 - DNA, 21 bp, Artificial Sequence
  Description of Artificial Sequence Synthetic oligonucleotide
ugucaugacugggcacauutt

SEQ ID NO: 171 - DNA, 21 bp, Artificial Sequence
  Description of Artificial Sequence Synthetic oligonucleotide
ugcccagucaugacauuuctt

SEQ ID NO: 172 - DNA, 21 bp, Artificial Sequence
  Description of Artificial Sequence Synthetic oligonucleotide
gaaaugucaugacugggcatt

SEQ ID NO: 173 - DNA, 21 bp, Artificial Sequence
  Description of Artificial Sequence Synthetic oligonucleotide
cagucaugacauuucucaatt

SEQ ID NO: 174 - DNA, 21 bp, Artificial Sequence
  Description of Artificial Sequence Synthetic oligonucleotide
uugagaaaugucaugacugtt

SEQ ID NO: 175 - DNA, 21 bp, Artificial Sequence
  Description of Artificial Sequence Synthetic oligonucleotide
aaguuuuuacaguguaucutt

SEQ ID NO: 176 - DNA, 21 bp, Artificial Sequence
  Description of Artificial Sequence Synthetic oligonucleotide
agauacacuguaaaaacuutt

SEQ ID NO: 177 - DNA, 21 bp, Artificial Sequence
  Description of Artificial Sequence Synthetic oligonucleotide
caguguaucucgaagucuutt

SEQ ID NO: 178 - DNA, 21 bp, Artificial Sequence
  Description of Artificial Sequence Synthetic oligonucleotide
aagacuucgagauacacugtt

SEQ ID NO: 179 - DNA, 21 bp, Artificial Sequence
  Description of Artificial Sequence Synthetic oligonucleotide
guaucucgaagucuuccautt

SEQ ID NO: 180 - DNA, 21 bp, Artificial Sequence
  Description of Artificial Sequence Synthetic oligonucleotide
auggaagacuucgagauactt

SEQ ID NO: 181 - DNA, 21 bp, Artificial Sequence
  Description of Artificial Sequence Synthetic oligonucleotide
cagcagugauugaaguauctt

SEQ ID NO: 182 - DNA, 21 bp, Artificial Sequence
  Description of Artificial Sequence Synthetic oligonucleotide
gauacuucaaucacugcugtt

SEQ ID NO: 183 - DNA, 21 bp, Artificial Sequence
  Description of Artificial Sequence Synthetic oligonucleotide
ccacucagcauuucggugctt

SEQ ID NO: 184 - DNA, 21 bp, Artificial Sequence
  Description of Artificial Sequence Synthetic oligonucleotide
gcaccgaaaugcugaguggtt

SEQ ID NO: 185 - DNA, 21 bp, Artificial Sequence
  Description of Artificial Sequence Synthetic oligonucleotide
ucagcauuucggugcuucctt

SEQ ID NO: 186 - DNA, 21 bp, Artificial Sequence
  Description of Artificial Sequence Synthetic oligonucleotide
ggaagcaccgaaaugcugatt

SEQ ID NO: 187 - DNA, 21 bp, Artificial Sequence
  Description of Artificial Sequence Synthetic oligonucleotide
aagugaauacaugguagcatt

SEQ ID NO: 188 - DNA, 21 bp, Artificial Sequence
  Description of Artificial Sequence Synthetic oligonucleotide
ugcuaccauguauucacuutt

SEQ ID NO: 189 - DNA, 21 bp, Artificial Sequence
  Description of Artificial Sequence Synthetic oligonucleotide
gaauacaugguagcagggutt

SEQ ID NO: 190 - DNA, 21 bp, Artificial Sequence
  Description of Artificial Sequence Synthetic oligonucleotide
acccugcuaccauguauuctt

SEQ ID NO: 191 - DNA, 21 bp, Artificial Sequence
  Description of Artificial Sequence Synthetic oligonucleotide
ugguagcagggucuuugugtt

SEQ ID NO: 192 - DNA, 21 bp, Artificial Sequence
  Description of Artificial Sequence Synthetic oligonucleotide
cacaaagacccugcuaccatt

SEQ ID NO: 193 - DNA, 21 bp, Artificial Sequence
  Description of Artificial Sequence Synthetic oligonucleotide
uuuuguggcuucaaucuactt

SEQ ID NO: 194 - DNA, 21 bp, Artificial Sequence
  Description of Artificial Sequence Synthetic oligonucleotide
guagauugaagccacaaaatt

SEQ ID NO: 195 - DNA, 21 bp, Artificial Sequence
  Description of Artificial Sequence Synthetic oligonucleotide
uuuguggcuucaaucuacgtt

SEQ ID NO: 196 - DNA, 21 bp, Artificial Sequence
  Description of Artificial Sequence Synthetic oligonucleotide
cguagauugaagccacaaatt

SEQ ID NO: 197 - DNA, 21 bp, Artificial Sequence
  Description of Artificial Sequence Synthetic oligonucleotide
aauuaaaaacaccuaagugtt

SEQ ID NO: 198 - DNA, 21 bp, Artificial Sequence
  Description of Artificial Sequence Synthetic oligonucleotide
cacuuagguguuuuuaauutt

SEQ ID NO: 199 - DNA, 21 bp, Artificial Sequence
  Description of Artificial Sequence Synthetic oligonucleotide
auuaaaaacaccuaagugatt

SEQ ID NO: 200 - DNA, 21 bp, Artificial Sequence
  Description of Artificial Sequence Synthetic oligonucleotide
ucacuuagguguuuuuaautt

SEQ ID NO: 201 - DNA, 21 bp, Artificial Sequence
  Description of Artificial Sequence Synthetic oligonucleotide
aaacaccuaagugacuacctt

SEQ ID NO: 202 - DNA, 21 bp, Artificial Sequence
  Description of Artificial Sequence Synthetic oligonucleotide
gguagucacuuagguguuutt

SEQ ID NO: 203 - DNA, 21 bp, Artificial Sequence
  Description of Artificial Sequence Synthetic oligonucleotide
gacuaccacuuauuucuaatt

SEQ ID NO: 204 - DNA, 21 bp, Artificial Sequence
  Description of Artificial Sequence Synthetic oligonucleotide
uuagaaauaagugguaguctt

SEQ ID NO: 205 - DNA, 21 bp, Artificial Sequence
  Description of Artificial Sequence Synthetic oligonucleotide
uuauuucuaaauccucacutt

SEQ ID NO: 206 - DNA, 21 bp, Artificial Sequence
  Description of Artificial Sequence Synthetic oligonucleotide
agugaggauuuagaaauaatt

SEQ ID NO: 207 - DNA, 21 bp, Artificial Sequence
  Description of Artificial Sequence Synthetic oligonucleotide
cagaaguuguuagugauuutt

SEQ ID NO: 208 - DNA, 21 bp, Artificial Sequence
  Description of Artificial Sequence Synthetic oligonucleotide
aaaucacuaacaacuucugtt

SEQ ID NO: 209 - DNA, 21 bp, Artificial Sequence
  Description of Artificial Sequence Synthetic oligonucleotide
uuauaagauuuuuaggugutt

SEQ ID NO: 210 - DNA, 21 bp, Artificial Sequence
  Description of Artificial Sequence Synthetic oligonucleotide
acaccuaaaaaucuuauaatt

SEQ ID NO: 211 - DNA, 21 bp, Artificial Sequence
  Description of Artificial Sequence Synthetic oligonucleotide
uauaagauuuuuagguguctt

SEQ ID NO: 212 - DNA, 21 bp, Artificial Sequence
  Description of Artificial Sequence Synthetic oligonucleotide
gacaccuaaaaaucuuauatt

SEQ ID NO: 213 - DNA, 21 bp, Artificial Sequence
  Description of Artificial Sequence Synthetic oligonucleotide
uaagaauaaugacguauugtt

SEQ ID NO: 214 - DNA, 21 bp, Artificial Sequence
  Description of Artificial Sequence Synthetic oligonucleotide
caauacgucauuauucuuatt

SEQ ID NO: 215 - DNA, 21 bp, Artificial Sequence
  Description of Artificial Sequence Synthetic oligonucleotide
aacuaugcaccuauaaauatt

SEQ ID NO: 216 - DNA, 21 bp, Artificial Sequence
  Description of Artificial Sequence Synthetic oligonucleotide
uauuuauaggugcauaguutt

SEQ ID NO: 217 - DNA, 21 bp, Artificial Sequence
  Description of Artificial Sequence Synthetic oligonucleotide
aaauuuuaccauuuugcgatt

SEQ ID NO: 218 - DNA, 21 bp, Artificial Sequence
  Description of Artificial Sequence Synthetic oligonucleotide
ucgcaaaaugguaaaauuutt

SEQ ID NO: 219 - DNA, 21 bp, Artificial Sequence
  Description of Artificial Sequence Synthetic oligonucleotide
aauuuuaccauuuugcgautt

SEQ ID NO: 220 - DNA, 21 bp, Artificial Sequence
  Description of Artificial Sequence Synthetic oligonucleotide
aucgcaaaaugguaaaauutt

SEQ ID NO: 221 - DNA, 21 bp, Artificial Sequence
  Description of Artificial Sequence Synthetic oligonucleotide
uuuuaccauuuugcgaugutt

SEQ ID NO: 222 - DNA, 21 bp, Artificial Sequence
  Description of Artificial Sequence Synthetic oligonucleotide
acaucgcaaaaugguaaaatt

SEQ ID NO: 223 - DNA, 21 bp, Artificial Sequence
  Description of Artificial Sequence Synthetic oligonucleotide
uugcgauguguuuuauucatt

SEQ ID NO: 224 - DNA, 21 bp, Artificial Sequence
  Description of Artificial Sequence Synthetic oligonucleotide
ugaauaaaacacaucgcaatt

SEQ ID NO: 225 - DNA, 21 bp, Artificial Sequence
  Description of Artificial Sequence Synthetic oligonucleotide
ugcgauguguuuuauucactt

SEQ ID NO: 226 - DNA, 21 bp, Artificial Sequence
  Description of Artificial Sequence Synthetic oligonucleotide
gugaauaaaacacaucgcatt

SEQ ID NO: 227 - DNA, 21 bp, Artificial Sequence
  Description of Artificial Sequence Synthetic oligonucleotide
cguuaucucauugcaaaaatt

SEQ ID NO: 228 - DNA, 21 bp, Artificial Sequence
  Description of Artificial Sequence Synthetic oligonucleotide
uuuuugcaaugagauaacgtt

SEQ ID NO: 229 - DNA, 21 bp, Artificial Sequence
  Description of Artificial Sequence Synthetic oligonucleotide
uaucccaucucacuuuaautt

SEQ ID NO: 230 - DNA, 21 bp, Artificial Sequence
  Description of Artificial Sequence Synthetic oligonucleotide
auuaaagugagaugggauatt

SEQ ID NO: 231 - DNA, 21 bp, Artificial Sequence
  Description of Artificial Sequence Synthetic oligonucleotide
aucccaucucacuuuaauatt

SEQ ID NO: 232 - DNA, 21 bp, Artificial Sequence
  Description of Artificial Sequence Synthetic oligonucleotide
uauuaaagugagaugggautt

SEQ ID NO: 233 - DNA, 21 bp, Artificial Sequence
  Description of Artificial Sequence Synthetic oligonucleotide
caucucacuuuaauaauaatt

SEQ ID NO: 234 - DNA, 21 bp, Artificial Sequence
  Description of Artificial Sequence Synthetic oligonucleotide
uuauuauuaaagugagaugtt

SEQ ID NO: 235 - DNA, 21 bp, Artificial Sequence
  Description of Artificial Sequence Synthetic oligonucleotide
aaaucaugcuuauaagcaatt

SEQ ID NO: 236 - DNA, 21 bp, Artificial Sequence
  Description of Artificial Sequence Synthetic oligonucleotide
uugcuuauaagcaugauuutt

SEQ ID NO: 237 - DNA, 21 bp, Artificial Sequence
  Description of Artificial Sequence Synthetic oligonucleotide
augcuuauaagcaacaugatt

SEQ ID NO: 238 - DNA, 21 bp, Artificial Sequence
  Description of Artificial Sequence Synthetic oligonucleotide
ucauguugcuuauaagcautt

SEQ ID NO: 239 - DNA, 21 bp, Artificial Sequence
  Description of Artificial Sequence Synthetic oligonucleotide
agaacugacacaaaggacatt

SEQ ID NO: 240 - DNA, 21 bp, Artificial Sequence
  Description of Artificial Sequence Synthetic oligonucleotide
uguccuuugugucaguucutt

SEQ ID NO: 241 - DNA, 21 bp, Artificial Sequence
  Description of Artificial Sequence Synthetic oligonucleotide
auaaaguuauuaauagccatt

SEQ ID NO: 242 - DNA, 21 bp, Artificial Sequence
  Description of Artificial Sequence Synthetic oligonucleotide
uggcuauuaauaacuuuautt

SEQ ID NO: 243 - DNA, 21 bp, Artificial Sequence
  Description of Artificial Sequence Synthetic oligonucleotide
aaaguuauuaauagccauutt

SEQ ID NO: 244 - DNA, 21 bp, Artificial Sequence
  Description of Artificial Sequence Synthetic oligonucleotide
aauggcuauuaauaacuuutt

SEQ ID NO: 245 - DNA, 21 bp, Artificial Sequence
  Description of Artificial Sequence Synthetic oligonucleotide
ggauguauucaugaaaggatt

SEQ ID NO: 246 - DNA, 21 bp, Artificial Sequence
  Description of Artificial Sequence Synthetic oligonucleotide
uccuuucaugaauacaucctt

SEQ ID NO: 247 - DNA, 21 bp, Artificial Sequence
  Description of Artificial Sequence Synthetic oligonucleotide
guucucuauguaggcuccatt

SEQ ID NO: 248 - DNA, 21 bp, Artificial Sequence
  Description of Artificial Sequence Synthetic oligonucleotide
uggagccuacauagagaactt

SEQ ID NO: 249 - DNA, 21 bp, Artificial Sequence
  Description of Artificial Sequence Synthetic oligonucleotide
guuccauccuguacaagugtt

SEQ ID NO: 250 - DNA, 21 bp, Artificial Sequence
  Description of Artificial Sequence Synthetic oligonucleotide
cacuuguacaggauggaactt

SEQ ID NO: 251 - DNA, 21 bp, Artificial Sequence
  Description of Artificial Sequence Synthetic oligonucleotide
cuaaauccucacuauuuuutt

SEQ ID NO: 252 - DNA, 21 bp, Artificial Sequence
  Description of Artificial Sequence Synthetic oligonucleotide
aaaaauagugaggauuuagtt

SEQ ID NO: 253 - DNA, 21 bp, Artificial Sequence
  Description of Artificial Sequence Synthetic oligonucleotide
cuauguaggcuccaaaacctt

SEQ ID NO: 254 - DNA, 21 bp, Artificial Sequence
  Description of Artificial Sequence Synthetic oligonucleotide
gguuuuggagccuacauagtt

SEQ ID NO: 255 - DNA, 21 bp, Artificial Sequence
  Description of Artificial Sequence Synthetic oligonucleotide
ggugcaugguguggcaacatt

SEQ ID NO: 256 - DNA, 21 bp, Artificial Sequence
  Description of Artificial Sequence Synthetic oligonucleotide
uguugccacaccaugcacctt

SEQ ID NO: 257 - DNA, 21 bp, Artificial Sequence
  Description of Artificial Sequence Synthetic oligonucleotide
ugagaucugcugacagaugtt

SEQ ID NO: 258 - DNA, 21 bp, Artificial Sequence
  Description of Artificial Sequence Synthetic oligonucleotide
caucugucagcagaucucatt

SEQ ID NO: 259 - DNA, 21 bp, Artificial Sequence
  Description of Artificial Sequence Synthetic oligonucleotide
cccacucagcauuucggugtt

SEQ ID NO: 260 - DNA, 21 bp, Artificial Sequence
  Description of Artificial Sequence Synthetic oligonucleotide
caccgaaaugcugagugggtt

SEQ ID NO: 261 - DNA, 21 bp, Artificial Sequence
  Description of Artificial Sequence Synthetic oligonucleotide
caugguagcagggucuuugtt

SEQ ID NO: 262 - DNA, 21 bp, Artificial Sequence
  Description of Artificial Sequence Synthetic oligonucleotide
caaagacccugcuaccaugtt

SEQ ID NO: 263 - DNA, 21 bp, Artificial Sequence
  Description of Artificial Sequence Synthetic oligonucleotide
agaauuaaaauaaaacguutt

SEQ ID NO: 264 - DNA, 21 bp, Artificial Sequence
  Description of Artificial Sequence Synthetic oligonucleotide
aacguuuuauuuuaauucutt

SEQ ID NO: 265 - DNA, 21 bp, Artificial Sequence
  Description of Artificial Sequence Synthetic oligonucleotide
aucucgaagucuuccaucatt

SEQ ID NO: 266 - DNA, 21 bp, Artificial Sequence
  Description of Artificial Sequence Synthetic oligonucleotide
ugauggaagacuucgagautt

SEQ ID NO: 267 - DNA, 21 bp, Artificial Sequence
  Description of Artificial Sequence Synthetic oligonucleotide
gugagcaugaaacuaugcatt

SEQ ID NO: 268 - DNA, 21 bp, Artificial Sequence
  Description of Artificial Sequence Synthetic oligonucleotide
ugcauaguuucaugcucactt

SEQ ID NO: 269 - RNA, 19 nt, Artificial Sequence
  Description of Artificial Sequence Synthetic oligonucleotide
guggcuucaaucuacgaug

SEQ ID NO: 270 - RNA, 19 nt, Artificial Sequence
  Description of Artificial Sequence Synthetic oligonucleotide
caucguagauugaagccac

SEQ ID NO: 271 - RNA, 19 nt, Artificial Sequence
  Description of Artificial Sequence Synthetic oligonucleotide
uuuuuacaguguaucucga

SEQ ID NO: 272 - RNA, 19 nt, Artificial Sequence
  Description of Artificial Sequence Synthetic oligonucleotide
ucgagauacacuguaaaaa

SEQ ID NO: 273 - RNA, 19 nt, Artificial Sequence
  Description of Artificial Sequence Synthetic oligonucleotide
guaucaagacuacgaaccu

SEQ ID NO: 274 - RNA, 19 nt, Artificial Sequence
  Description of Artificial Sequence Synthetic oligonucleotide
agguucguagucuugauac

SEQ ID NO: 275 - RNA, 19 nt, Artificial Sequence
  Description of Artificial Sequence Synthetic oligonucleotide
uuuuacaguguaucucgaa

SEQ ID NO: 276 - RNA, 19 nt, Artificial Sequence
  Description of Artificial Sequence Synthetic oligonucleotide
uucgagauacacuguaaaa

SEQ ID NO: 277 - RNA, 19 nt, Artificial Sequence
  Description of Artificial Sequence Synthetic oligonucleotide
acauuaacccuacacucgg

SEQ ID NO: 278 - RNA, 19 nt, Artificial Sequence
  Description of Artificial Sequence Synthetic oligonucleotide
ccgaguguaggguuaaugu

SEQ ID NO: 279 - RNA, 19 nt, Artificial Sequence
  Description of Artificial Sequence Synthetic oligonucleotide
uguggcuucaaucuacgau

SEQ ID NO: 280 - RNA, 19 nt, Artificial Sequence
  Description of Artificial Sequence Synthetic oligonucleotide
aucguagauugaagccaca

SEQ ID NO: 281 - RNA, 19 nt, Artificial Sequence
  Description of Artificial Sequence Synthetic oligonucleotide
cucagcauuucggugcuuc

SEQ ID NO: 282 - RNA, 19 nt, Artificial Sequence
  Description of Artificial Sequence Synthetic oligonucleotide
gaagcaccgaaaugcugag

SEQ ID NO: 283 - RNA, 19 nt, Artificial Sequence
  Description of Artificial Sequence Synthetic oligonucleotide
uuaacccuacacucggaau

SEQ ID NO: 284 - RNA, 19 nt, Artificial Sequence
  Description of Artificial Sequence Synthetic oligonucleotide
auuccgaguguaggguuaa

SEQ ID NO: 285 - RNA, 19 nt, Artificial Sequence
  Description of Artificial Sequence Synthetic oligonucleotide
uuacaguguaucucgaagu

SEQ ID NO: 286 - RNA, 19 nt, Artificial Sequence
  Description of Artificial Sequence Synthetic oligonucleotide
acuucgagauacacuguaa

SEQ ID NO: 287 - RNA, 19 nt, Artificial Sequence
  Description of Artificial Sequence Synthetic oligonucleotide
acaguguaucucgaagucu

SEQ ID NO: 288 - RNA, 19 nt, Artificial Sequence
  Description of Artificial Sequence Synthetic oligonucleotide
agacuucgagauacacugu

SEQ ID NO: 289 - RNA, 19 nt, Artificial Sequence
  Description of Artificial Sequence Synthetic oligonucleotide
gcuucaaucuacgauguua

SEQ ID NO: 290 - RNA, 19 nt, Artificial Sequence
  Description of Artificial Sequence Synthetic oligonucleotide
uaacaucguagauugaagc

SEQ ID NO: 291 - RNA, 19 nt, Artificial Sequence
  Description of Artificial Sequence Synthetic oligonucleotide
auuaacccuacacucggaa

SEQ ID NO: 292 - RNA, 19 nt, Artificial Sequence
  Description of Artificial Sequence Synthetic oligonucleotide
uuccgaguguaggguuaau

SEQ ID NO: 293 - RNA, 19 nt, Artificial Sequence
  Description of Artificial Sequence Synthetic oligonucleotide
uuaaugauacugucuaaga

SEQ ID NO: 294 - RNA, 19 nt, Artificial Sequence
  Description of Artificial Sequence Synthetic oligonucleotide
ucuuagacaguaucauuaa

SEQ ID NO: 295 - RNA, 19 nt, Artificial Sequence
  Description of Artificial Sequence Synthetic oligonucleotide
aucaagacuacgaaccuga

SEQ ID NO: 296 - RNA, 19 nt, Artificial Sequence
  Description of Artificial Sequence Synthetic oligonucleotide
ucagguucguagucuugau

SEQ ID NO: 297 - RNA, 19 nt, Artificial Sequence
  Description of Artificial Sequence Synthetic oligonucleotide
agugaauacaugguagcag

SEQ ID NO: 298 - RNA, 19 nt, Artificial Sequence
  Description of Artificial Sequence Synthetic oligonucleotide
cugcuaccauguauucacu

SEQ ID NO: 299 - RNA, 19 nt, Artificial Sequence
  Description of Artificial Sequence Synthetic oligonucleotide
aauacaugguagcaggguc

SEQ ID NO: 300 - RNA, 19 nt, Artificial Sequence
  Description of Artificial Sequence Synthetic oligonucleotide
gacccugcuaccauguauu

SEQ ID NO: 301 - RNA, 19 nt, Artificial Sequence
  Description of Artificial Sequence Synthetic oligonucleotide
cuaagugacuaccacuuau

SEQ ID NO: 302 - RNA, 19 nt, Artificial Sequence
  Description of Artificial Sequence Synthetic oligonucleotide
auaagugguagucacuuag

SEQ ID NO: 303 - RNA, 19 nt, Artificial Sequence
  Description of Artificial Sequence Synthetic oligonucleotide
agcaugaaacuaugcaccu

SEQ ID NO: 304 - RNA, 19 nt, Artificial Sequence
  Description of Artificial Sequence Synthetic oligonucleotide
aggugcauaguuucaugcu

SEQ ID NO: 305 - RNA, 19 nt, Artificial Sequence
  Description of Artificial Sequence Synthetic oligonucleotide
uuguggcuucaaucuacga

SEQ ID NO: 306 - RNA, 19 nt, Artificial Sequence
  Description of Artificial Sequence Synthetic oligonucleotide
ucguagauugaagccacaa

SEQ ID NO: 307 - RNA, 19 nt, Artificial Sequence
  Description of Artificial Sequence Synthetic oligonucleotide
ggcuucaaucuacgauguu

SEQ ID NO: 308 - RNA, 19 nt, Artificial Sequence
  Description of Artificial Sequence Synthetic oligonucleotide
aacaucguagauugaagcc

SEQ ID NO: 309 - RNA, 19 nt, Artificial Sequence
  Description of Artificial Sequence Synthetic oligonucleotide
auaaugacguauugugaaa

SEQ ID NO: 310 - RNA, 19 nt, Artificial Sequence
  Description of Artificial Sequence Synthetic oligonucleotide
uuucacaauacgucauuau

SEQ ID NO: 311 - RNA, 19 nt, Artificial Sequence
  Description of Artificial Sequence Synthetic oligonucleotide
agugauuugcuaucauaua

SEQ ID NO: 312 - RNA, 19 nt, Artificial Sequence
  Description of Artificial Sequence Synthetic oligonucleotide
uauaugauagcaaaucacu

SEQ ID NO: 313 - RNA, 19 nt, Artificial Sequence
  Description of Artificial Sequence Synthetic oligonucleotide
caaucuacgauguuaaaac

SEQ ID NO: 314 - RNA, 19 nt, Artificial Sequence
  Description of Artificial Sequence Synthetic oligonucleotide
guuuuaacaucguagauug

SEQ ID NO: 315 - RNA, 19 nt, Artificial Sequence
  Description of Artificial Sequence Synthetic oligonucleotide
acguauugugaaauuuguu

SEQ ID NO: 316 - RNA, 19 nt, Artificial Sequence
  Description of Artificial Sequence Synthetic oligonucleotide
aacaaauuucacaauacgu

SEQ ID NO: 317 - RNA, 19 nt, Artificial Sequence
  Description of Artificial Sequence Synthetic oligonucleotide
gacguauugugaaauuugu

SEQ ID NO: 318 - RNA, 19 nt, Artificial Sequence
  Description of Artificial Sequence Synthetic oligonucleotide
acaaauuucacaauacguc

SEQ ID NO: 319 - RNA, 19 nt, Artificial Sequence
  Description of Artificial Sequence Synthetic oligonucleotide
acgggugugacagcaguag

SEQ ID NO: 320 - RNA, 19 nt, Artificial Sequence
  Description of Artificial Sequence Synthetic oligonucleotide
cuacugcugucacacccgu

SEQ ID NO: 321 - RNA, 19 nt, Artificial Sequence
  Description of Artificial Sequence Synthetic oligonucleotide
gguaucaagacuacgaacc

SEQ ID NO: 322 - RNA, 19 nt, Artificial Sequence
  Description of Artificial Sequence Synthetic oligonucleotide
gguucguagucuugauacc

SEQ ID NO: 323 - RNA, 19 nt, Artificial Sequence
  Description of Artificial Sequence Synthetic oligonucleotide
ugucuaagaauaaugacgu

SEQ ID NO: 324 - RNA, 19 nt, Artificial Sequence
  Description of Artificial Sequence Synthetic oligonucleotide
acgucauuauucuuagaca

SEQ ID NO: 325 - RNA, 19 nt, Artificial Sequence
  Description of Artificial Sequence Synthetic oligonucleotide
aacccuacacucggaauuc

SEQ ID NO: 326 - RNA, 19 nt, Artificial Sequence
  Description of Artificial Sequence Synthetic oligonucleotide
gaauuccgaguguaggguu

SEQ ID NO: 327 - RNA, 19 nt, Artificial Sequence
  Description of Artificial Sequence Synthetic oligonucleotide
aaaagaggguguucucuau

SEQ ID NO: 328 - RNA, 19 nt, Artificial Sequence
  Description of Artificial Sequence Synthetic oligonucleotide
auagagaacacccucuuuu

SEQ ID NO: 329 - RNA, 19 nt, Artificial Sequence
  Description of Artificial Sequence Synthetic oligonucleotide
ggguguucucuauguaggc

SEQ ID NO: 330 - RNA, 19 nt, Artificial Sequence
  Description of Artificial Sequence Synthetic oligonucleotide
gccuacauagagaacaccc

SEQ ID NO: 331 - RNA, 19 nt, Artificial Sequence
  Description of Artificial Sequence Synthetic oligonucleotide
ucucuauguaggcuccaaa

SEQ ID NO: 332 - RNA, 19 nt, Artificial Sequence
  Description of Artificial Sequence Synthetic oligonucleotide
uuuggagccuacauagaga

SEQ ID NO: 333 - RNA, 19 nt, Artificial Sequence
  Description of Artificial Sequence Synthetic oligonucleotide
gaaggguaucaagacuacg

SEQ ID NO: 334 - RNA, 19 nt, Artificial Sequence
  Description of Artificial Sequence Synthetic oligonucleotide
cguagucuugauacccuuc

SEQ ID NO: 335 - RNA, 19 nt, Artificial Sequence
  Description of Artificial Sequence Synthetic oligonucleotide
aaggguaucaagacuacga

SEQ ID NO: 336 - RNA, 19 nt, Artificial Sequence
  Description of Artificial Sequence Synthetic oligonucleotide
ucguagucuugauacccuu

SEQ ID NO: 337 - RNA, 19 nt, Artificial Sequence
  Description of Artificial Sequence Synthetic oligonucleotide
ggguaucaagacuacgaac

SEQ ID NO: 338 - RNA, 19 nt, Artificial Sequence
  Description of Artificial Sequence Synthetic oligonucleotide
guucguagucuugauaccc

SEQ ID NO: 339 - RNA, 19 nt, Artificial Sequence
  Description of Artificial Sequence Synthetic oligonucleotide
gaagccuaagaaauaucuu

SEQ ID NO: 340 - RNA, 19 nt, Artificial Sequence
  Description of Artificial Sequence Synthetic oligonucleotide
aagauauuucuuaggcuuc

SEQ ID NO: 341 - RNA, 19 nt, Artificial Sequence
  Description of Artificial Sequence Synthetic oligonucleotide
uuuacaguguaucucgaag

SEQ ID NO: 342 - RNA, 19 nt, Artificial Sequence
  Description of Artificial Sequence Synthetic oligonucleotide
cuucgagauacacuguaaa

SEQ ID NO: 343 - RNA, 19 nt, Artificial Sequence
  Description of Artificial Sequence Synthetic oligonucleotide
uacaguguaucucgaaguc

SEQ ID NO: 344 - RNA, 19 nt, Artificial Sequence
  Description of Artificial Sequence Synthetic oligonucleotide
gacuucgagauacacugua

SEQ ID NO: 345 - RNA, 19 nt, Artificial Sequence
  Description of Artificial Sequence Synthetic oligonucleotide
cacucagcauuucggugcu

SEQ ID NO: 346 - RNA, 19 nt, Artificial Sequence
  Description of Artificial Sequence Synthetic oligonucleotide
agcaccgaaaugcugagug

SEQ ID NO: 347 - RNA, 19 nt, Artificial Sequence
  Description of Artificial Sequence Synthetic oligonucleotide
uggcuucaaucuacgaugu

SEQ ID NO: 348 - RNA, 19 nt, Artificial Sequence
  Description of Artificial Sequence Synthetic oligonucleotide
acaucguagauugaagcca

SEQ ID NO: 349 - RNA, 19 nt, Artificial Sequence
  Description of Artificial Sequence Synthetic oligonucleotide
cuucaaucuacgauguuaa

SEQ ID NO: 350 - RNA, 19 nt, Artificial Sequence
  Description of Artificial Sequence Synthetic oligonucleotide
uuaacaucguagauugaag

SEQ ID NO: 351 - RNA, 19 nt, Artificial Sequence
  Description of Artificial Sequence Synthetic oligonucleotide
aaucuacgauguuaaaaca

SEQ ID NO: 352 - RNA, 19 nt, Artificial Sequence
  Description of Artificial Sequence Synthetic oligonucleotide
uguuuuaacaucguagauu

SEQ ID NO: 353 - RNA, 19 nt, Artificial Sequence
  Description of Artificial Sequence Synthetic oligonucleotide
aacaccuaagugacuacca

SEQ ID NO: 354 - RNA, 19 nt, Artificial Sequence
  Description of Artificial Sequence Synthetic oligonucleotide
ugguagucacuuagguguu

SEQ ID NO: 355 - RNA, 19 nt, Artificial Sequence
  Description of Artificial Sequence Synthetic oligonucleotide
uaagugacuaccacuuauu

SEQ ID NO: 356 - RNA, 19 nt, Artificial Sequence
  Description of Artificial Sequence Synthetic oligonucleotide
aauaagugguagucacuua

SEQ ID NO: 357 - RNA, 19 nt, Artificial Sequence
  Description of Artificial Sequence Synthetic oligonucleotide
uguucagaaguuguuagug

SEQ ID NO: 358 - RNA, 19 nt, Artificial Sequence
  Description of Artificial Sequence Synthetic oligonucleotide
cacuaacaacuucugaaca

SEQ ID NO: 359 - RNA, 19 nt, Artificial Sequence
  Description of Artificial Sequence Synthetic oligonucleotide
aaguuguuagugauuugcu

SEQ ID NO: 360 - RNA, 19 nt, Artificial Sequence
  Description of Artificial Sequence Synthetic oligonucleotide
agcaaaucacuaacaacuu

SEQ ID NO: 361 - RNA, 19 nt, Artificial Sequence
  Description of Artificial Sequence Synthetic oligonucleotide
auaagauuuuuaggugucu

SEQ ID NO: 362 - RNA, 19 nt, Artificial Sequence
  Description of Artificial Sequence Synthetic oligonucleotide
agacaccuaaaaaucuuau

SEQ ID NO: 363 - RNA, 19 nt, Artificial Sequence
  Description of Artificial Sequence Synthetic oligonucleotide
aaugacguauugugaaauu

SEQ ID NO: 364 - RNA, 19 nt, Artificial Sequence
  Description of Artificial Sequence Synthetic oligonucleotide
aauuucacaauacgucauu

SEQ ID NO: 365 - RNA, 19 nt, Artificial Sequence
  Description of Artificial Sequence Synthetic oligonucleotide
gcaugaaacuaugcaccua

SEQ ID NO: 366 - RNA, 19 nt, Artificial Sequence
  Description of Artificial Sequence Synthetic oligonucleotide
uaggugcauaguuucaugc

SEQ ID NO: 367 - RNA, 19 nt, Artificial Sequence
  Description of Artificial Sequence Synthetic oligonucleotide
uaugcaccuauaaauacua

SEQ ID NO: 368 - RNA, 19 nt, Artificial Sequence
  Description of Artificial Sequence Synthetic oligonucleotide
uaguauuuauaggugcaua

SEQ ID NO: 369 - RNA, 19 nt, Artificial Sequence
  Description of Artificial Sequence Synthetic oligonucleotide
uaccauuuugcgauguguu

SEQ ID NO: 370 - RNA, 19 nt, Artificial Sequence
  Description of Artificial Sequence Synthetic oligonucleotide
aacacaucgcaaaauggua

SEQ ID NO: 371 - RNA, 19 nt, Artificial Sequence
  Description of Artificial Sequence Synthetic oligonucleotide
auuuuuaucccaucucacu

SEQ ID NO: 372 - RNA, 19 nt, Artificial Sequence
  Description of Artificial Sequence Synthetic oligonucleotide
agugagaugggauaaaaau

SEQ ID NO: 373 - RNA, 19 nt, Artificial Sequence
  Description of Artificial Sequence Synthetic oligonucleotide
uuaucccaucucacuuuaa

SEQ ID NO: 374 - RNA, 19 nt, Artificial Sequence
  Description of Artificial Sequence Synthetic oligonucleotide
uuaaagugagaugggauaa

SEQ ID NO: 375 - RNA, 19 nt, Artificial Sequence
  Description of Artificial Sequence Synthetic oligonucleotide
uaacccuacacucggaauu

SEQ ID NO: 376 - RNA, 19 nt, Artificial Sequence
  Description of Artificial Sequence Synthetic oligonucleotide
aauuccgaguguaggguua

SEQ ID NO: 377 - RNA, 19 nt, Artificial Sequence
  Description of Artificial Sequence Synthetic oligonucleotide
aggguguucucuauguagg

SEQ ID NO: 378 - RNA, 19 nt, Artificial Sequence
  Description of Artificial Sequence Synthetic oligonucleotide
ccuacauagagaacacccu

SEQ ID NO: 379 - RNA, 19 nt, Artificial Sequence
  Description of Artificial Sequence Synthetic oligonucleotide
cucuauguaggcuccaaaa

SEQ ID NO: 380 - RNA, 19 nt, Artificial Sequence
  Description of Artificial Sequence Synthetic oligonucleotide
uuuuggagccuacauagag

SEQ ID NO: 381 - RNA, 19 nt, Artificial Sequence
  Description of Artificial Sequence Synthetic oligonucleotide
aaccaaacaggguguggca

SEQ ID NO: 382 - RNA, 19 nt, Artificial Sequence
  Description of Artificial Sequence Synthetic oligonucleotide
ugccacacccuguuugguu

SEQ ID NO: 383 - RNA, 19 nt, Artificial Sequence
  Description of Artificial Sequence Synthetic oligonucleotide
gaggguguucucuauguag

SEQ ID NO: 384 - RNA, 19 nt, Artificial Sequence
  Description of Artificial Sequence Synthetic oligonucleotide
cuacauagagaacacccuc

SEQ ID NO: 385 - RNA, 19 nt, Artificial Sequence
  Description of Artificial Sequence Synthetic oligonucleotide
uucucuauguaggcuccaa

SEQ ID NO: 386 - RNA, 19 nt, Artificial Sequence
  Description of Artificial Sequence Synthetic oligonucleotide
uuggagccuacauagagaa

SEQ ID NO: 387 - RNA, 19 nt, Artificial Sequence
  Description of Artificial Sequence Synthetic oligonucleotide
gugacaaauguuggaggag

SEQ ID NO: 388 - RNA, 19 nt, Artificial Sequence
  Description of Artificial Sequence Synthetic oligonucleotide
cuccuccaacauuugucac

SEQ ID NO: 389 - RNA, 19 nt, Artificial Sequence
  Description of Artificial Sequence Synthetic oligonucleotide
gucaaaaaggaccaguugg

SEQ ID NO: 390 - RNA, 19 nt, Artificial Sequence
  Description of Artificial Sequence Synthetic oligonucleotide
ccaacugguccuuuuugac

SEQ ID NO: 391 - RNA, 19 nt, Artificial Sequence
  Description of Artificial Sequence Synthetic oligonucleotide
ucaagacuacgaaccugaa

SEQ ID NO: 392 - RNA, 19 nt, Artificial Sequence
  Description of Artificial Sequence Synthetic oligonucleotide
uucagguucguagucuuga

SEQ ID NO: 393 - RNA, 19 nt, Artificial Sequence
  Description of Artificial Sequence Synthetic oligonucleotide
cagcauuucggugcuuccc

SEQ ID NO: 394 - RNA, 19 nt, Artificial Sequence
  Description of Artificial Sequence Synthetic oligonucleotide
gggaagcaccgaaaugcug

SEQ ID NO: 395 - RNA, 19 nt, Artificial Sequence
  Description of Artificial Sequence Synthetic oligonucleotide
guuguucagaaguuguuag

SEQ ID NO: 396 - RNA, 19 nt, Artificial Sequence
  Description of Artificial Sequence Synthetic oligonucleotide
cuaacaacuucugaacaac

SEQ ID NO: 397 - RNA, 19 nt, Artificial Sequence
  Description of Artificial Sequence Synthetic oligonucleotide
gaaguuguuagugauuugc

SEQ ID NO: 398 - RNA, 19 nt, Artificial Sequence
  Description of Artificial Sequence Synthetic oligonucleotide
gcaaaucacuaacaacuuc

SEQ ID NO: 399 - RNA, 19 nt, Artificial Sequence
  Description of Artificial Sequence Synthetic oligonucleotide
aguuguuagugauuugcua

SEQ ID NO: 400 - RNA, 19 nt, Artificial Sequence
  Description of Artificial Sequence Synthetic oligonucleotide
uagcaaaucacuaacaacu

SEQ ID NO: 401 - RNA, 19 nt, Artificial Sequence
  Description of Artificial Sequence Synthetic oligonucleotide
uuagugauuugcuaucaua

SEQ ID NO: 402 - RNA, 19 nt, Artificial Sequence
  Description of Artificial Sequence Synthetic oligonucleotide
uaugauagcaaaucacuaa

SEQ ID NO: 403 - RNA, 19 nt, Artificial Sequence
  Description of Artificial Sequence Synthetic oligonucleotide
gugauuugcuaucauauau

SEQ ID NO: 404 - RNA, 19 nt, Artificial Sequence
  Description of Artificial Sequence Synthetic oligonucleotide
auauaugauagcaaaucac

SEQ ID NO: 405 - RNA, 19 nt, Artificial Sequence
  Description of Artificial Sequence Synthetic oligonucleotide
uaaugacguauugugaaau

SEQ ID NO: 406 - RNA, 19 nt, Artificial Sequence
  Description of Artificial Sequence Synthetic oligonucleotide
auuucacaauacgucauua

SEQ ID NO: 407 - RNA, 19 nt, Artificial Sequence
  Description of Artificial Sequence Synthetic oligonucleotide
cuaugcaccuauaaauacu

SEQ ID NO: 408 - RNA, 19 nt, Artificial Sequence
  Description of Artificial Sequence Synthetic oligonucleotide
aguauuuauaggugcauag

SEQ ID NO: 409 - RNA, 19 nt, Artificial Sequence
  Description of Artificial Sequence Synthetic oligonucleotide
auuagccauggauguauuc

SEQ ID NO: 410 - RNA, 19 nt, Artificial Sequence
  Description of Artificial Sequence Synthetic oligonucleotide
gaauacauccauggcuaau

SEQ ID NO: 411 - RNA, 19 nt, Artificial Sequence
  Description of Artificial Sequence Synthetic oligonucleotide
uuagccauggauguauuca

SEQ ID NO: 412 - RNA, 19 nt, Artificial Sequence
  Description of Artificial Sequence Synthetic oligonucleotide
ugaauacauccauggcuaa

SEQ ID NO: 413 - RNA, 19 nt, Artificial Sequence
  Description of Artificial Sequence Synthetic oligonucleotide
gaaaggacuuucaaaggcc

SEQ ID NO: 414 - RNA, 19 nt, Artificial Sequence
  Description of Artificial Sequence Synthetic oligonucleotide
ggccuuugaaaguccuuuc

SEQ ID NO: 415 - RNA, 19 nt, Artificial Sequence
  Description of Artificial Sequence Synthetic oligonucleotide
caaaagaggguguucucua

SEQ ID NO: 416 - RNA, 19 nt, Artificial Sequence
  Description of Artificial Sequence Synthetic oligonucleotide
uagagaacacccucuuuug

SEQ ID NO: 417 - RNA, 19 nt, Artificial Sequence
  Description of Artificial Sequence Synthetic oligonucleotide
agaggguguucucuaugua

SEQ ID NO: 418 - RNA, 19 nt, Artificial Sequence
  Description of Artificial Sequence Synthetic oligonucleotide
uacauagagaacacccucu

SEQ ID NO: 419 - RNA, 19 nt, Artificial Sequence
  Description of Artificial Sequence Synthetic oligonucleotide
gguguucucuauguaggcu

SEQ ID NO: 420 - RNA, 19 nt, Artificial Sequence
  Description of Artificial Sequence Synthetic oligonucleotide
agccuacauagagaacacc

SEQ ID NO: 421 - RNA, 19 nt, Artificial Sequence
  Description of Artificial Sequence Synthetic oligonucleotide
guguucucuauguaggcuc

SEQ ID NO: 422 - RNA, 19 nt, Artificial Sequence
  Description of Artificial Sequence Synthetic oligonucleotide
gagccuacauagagaacac

SEQ ID NO: 423 - RNA, 19 nt, Artificial Sequence
  Description of Artificial Sequence Synthetic oligonucleotide
uguucucuauguaggcucc

SEQ ID NO: 424 - RNA, 19 nt, Artificial Sequence
  Description of Artificial Sequence Synthetic oligonucleotide
ggagccuacauagagaaca

SEQ ID NO: 425 - RNA, 19 nt, Artificial Sequence
  Description of Artificial Sequence Synthetic oligonucleotide
ggagcaguggugacgggug

SEQ ID NO: 426 - RNA, 19 nt, Artificial Sequence
  Description of Artificial Sequence Synthetic oligonucleotide
cacccgucaccacugcucc

SEQ ID NO: 427 - RNA, 19 nt, Artificial Sequence
  Description of Artificial Sequence Synthetic oligonucleotide
caguggugacgggugugac

SEQ ID NO: 428 - RNA, 19 nt, Artificial Sequence
  Description of Artificial Sequence Synthetic oligonucleotide
gucacacccgucaccacug

SEQ ID NO: 429 - RNA, 19 nt, Artificial Sequence
  Description of Artificial Sequence Synthetic oligonucleotide
uaucaagacuacgaaccug

SEQ ID NO: 430 - RNA, 19 nt, Artificial Sequence
  Description of Artificial Sequence Synthetic oligonucleotide
cagguucguagucuugaua

SEQ ID NO: 431 - RNA, 19 nt, Artificial Sequence
  Description of Artificial Sequence Synthetic oligonucleotide
cucccaguuucuugagauc

SEQ ID NO: 432 - RNA, 19 nt, Artificial Sequence
  Description of Artificial Sequence Synthetic oligonucleotide
gaucucaagaaacugggag

SEQ ID NO: 433 - RNA, 19 nt, Artificial Sequence
  Description of Artificial Sequence Synthetic oligonucleotide
gacagauguuccauccugu

SEQ ID NO: 434 - RNA, 19 nt, Artificial Sequence
  Description of Artificial Sequence Synthetic oligonucleotide
acaggauggaacaucuguc

SEQ ID NO: 435 - RNA, 19 nt, Artificial Sequence
  Description of Artificial Sequence Synthetic oligonucleotide
aaugugcccagucaugaca

SEQ ID NO: 436 - RNA, 19 nt, Artificial Sequence
  Description of Artificial Sequence Synthetic oligonucleotide
ugucaugacugggcacauu

SEQ ID NO: 437 - RNA, 19 nt, Artificial Sequence
  Description of Artificial Sequence Synthetic oligonucleotide
ugcccagucaugacauuuc

SEQ ID NO: 438 - RNA, 19 nt, Artificial Sequence
  Description of Artificial Sequence Synthetic oligonucleotide
gaaaugucaugacugggca

SEQ ID NO: 439 - RNA, 19 nt, Artificial Sequence
  Description of Artificial Sequence Synthetic oligonucleotide
cagucaugacauuucucaa

SEQ ID NO: 440 - RNA, 19 nt, Artificial Sequence
  Description of Artificial Sequence Synthetic oligonucleotide
uugagaaaugucaugacug

SEQ ID NO: 441 - RNA, 19 nt, Artificial Sequence
  Description of Artificial Sequence Synthetic oligonucleotide
aaguuuuuacaguguaucu

SEQ ID NO: 442 - RNA, 19 nt, Artificial Sequence
  Description of Artificial Sequence Synthetic oligonucleotide
agauacacuguaaaaacuu

SEQ ID NO: 443 - RNA, 19 nt, Artificial Sequence
  Description of Artificial Sequence Synthetic oligonucleotide
caguguaucucgaagucuu

SEQ ID NO: 444 - RNA, 19 nt, Artificial Sequence
  Description of Artificial Sequence Synthetic oligonucleotide
aagacuucgagauacacug

SEQ ID NO: 445 - RNA, 19 nt, Artificial Sequence
  Description of Artificial Sequence Synthetic oligonucleotide
guaucucgaagucuuccau

SEQ ID NO: 446 - RNA, 19 nt, Artificial Sequence
  Description of Artificial Sequence Synthetic oligonucleotide
auggaagacuucgagauac

SEQ ID NO: 447 - RNA, 19 nt, Artificial Sequence
  Description of Artificial Sequence Synthetic oligonucleotide
cagcagugauugaaguauc

SEQ ID NO: 448 - RNA, 19 nt, Artificial Sequence
  Description of Artificial Sequence Synthetic oligonucleotide
gauacuucaaucacugcug

SEQ ID NO: 449 - RNA, 19 nt, Artificial Sequence
  Description of Artificial Sequence Synthetic oligonucleotide
ccacucagcauuucggugc

SEQ ID NO: 450 - RNA, 19 nt, Artificial Sequence
  Description of Artificial Sequence Synthetic oligonucleotide
gcaccgaaaugcugagugg

SEQ ID NO: 451 - RNA, 19 nt, Artificial Sequence
  Description of Artificial Sequence Synthetic oligonucleotide
ucagcauuucggugcuucc

SEQ ID NO: 452 - RNA, 19 nt, Artificial Sequence
  Description of Artificial Sequence Synthetic oligonucleotide
ggaagcaccgaaaugcuga

SEQ ID NO: 453 - RNA, 19 nt, Artificial Sequence
  Description of Artificial Sequence Synthetic oligonucleotide
aagugaauacaugguagca

SEQ ID NO: 454 - RNA, 19 nt, Artificial Sequence
  Description of Artificial Sequence Synthetic oligonucleotide
ugcuaccauguauucacuu

SEQ ID NO: 455 - RNA, 19 nt, Artificial Sequence
  Description of Artificial Sequence Synthetic oligonucleotide
gaauacaugguagcagggu

SEQ ID NO: 456 - RNA, 19 nt, Artificial Sequence
  Description of Artificial Sequence Synthetic oligonucleotide
acccugcuaccauguauuc

SEQ ID NO: 457 - RNA, 19 nt, Artificial Sequence
  Description of Artificial Sequence Synthetic oligonucleotide
ugguagcagggucuuugug

SEQ ID NO: 458 - RNA, 19 nt, Artificial Sequence
  Description of Artificial Sequence Synthetic oligonucleotide
cacaaagacccugcuacca

SEQ ID NO: 459 - RNA, 19 nt, Artificial Sequence
  Description of Artificial Sequence Synthetic oligonucleotide
uuuuguggcuucaaucuac

SEQ ID NO: 460 - RNA, 19 nt, Artificial Sequence
  Description of Artificial Sequence Synthetic oligonucleotide
guagauugaagccacaaaa

SEQ ID NO: 461 - RNA, 19 nt, Artificial Sequence
  Description of Artificial Sequence Synthetic oligonucleotide
uuuguggcuucaaucuacg

SEQ ID NO: 462 - RNA, 19 nt, Artificial Sequence
  Description of Artificial Sequence Synthetic oligonucleotide
cguagauugaagccacaaa

SEQ ID NO: 463 - RNA, 19 nt, Artificial Sequence
  Description of Artificial Sequence Synthetic oligonucleotide
aauuaaaaacaccuaagug

SEQ ID NO: 464 - RNA, 19 nt, Artificial Sequence
  Description of Artificial Sequence Synthetic oligonucleotide
cacuuagguguuuuuaauu

SEQ ID NO: 465 - RNA, 19 nt, Artificial Sequence
  Description of Artificial Sequence Synthetic oligonucleotide
auuaaaaacaccuaaguga

SEQ ID NO: 466 - RNA, 19 nt, Artificial Sequence
  Description of Artificial Sequence Synthetic oligonucleotide
ucacuuagguguuuuuaau

SEQ ID NO: 467 - RNA, 19 nt, Artificial Sequence
  Description of Artificial Sequence Synthetic oligonucleotide
aaacaccuaagugacuacc

SEQ ID NO: 468 - RNA, 19 nt, Artificial Sequence
  Description of Artificial Sequence Synthetic oligonucleotide
gguagucacuuagguguuu

SEQ ID NO: 469 - RNA, 19 nt, Artificial Sequence
  Description of Artificial Sequence Synthetic oligonucleotide
gacuaccacuuauuucuaa

SEQ ID NO: 470 - RNA, 19 nt, Artificial Sequence
  Description of Artificial Sequence Synthetic oligonucleotide
uuagaaauaagugguaguc

SEQ ID NO: 471 - RNA, 19 nt, Artificial Sequence
  Description of Artificial Sequence Synthetic oligonucleotide
uuauuucuaaauccucacu

SEQ ID NO: 472 - RNA, 19 nt, Artificial Sequence
  Description of Artificial Sequence Synthetic oligonucleotide
agugaggauuuagaaauaa

SEQ ID NO: 473 - RNA, 19 nt, Artificial Sequence
  Description of Artificial Sequence Synthetic oligonucleotide
cagaaguuguuagugauuu

SEQ ID NO: 474 - RNA, 19 nt, Artificial Sequence
  Description of Artificial Sequence Synthetic oligonucleotide
aaaucacuaacaacuucug

SEQ ID NO: 475 - RNA, 19 nt, Artificial Sequence
  Description of Artificial Sequence Synthetic oligonucleotide
uuauaagauuuuuaggugu

SEQ ID NO: 476 - RNA, 19 nt, Artificial Sequence
  Description of Artificial Sequence Synthetic oligonucleotide
acaccuaaaaaucuuauaa

SEQ ID NO: 477 - RNA, 19 nt, Artificial Sequence
  Description of Artificial Sequence Synthetic oligonucleotide
uauaagauuuuuagguguc

SEQ ID NO: 478 - RNA, 19 nt, Artificial Sequence
  Description of Artificial Sequence Synthetic oligonucleotide
gacaccuaaaaaucuuaua

SEQ ID NO: 479 - RNA, 19 nt, Artificial Sequence
  Description of Artificial Sequence Synthetic oligonucleotide
uaagaauaaugacguauug

SEQ ID NO: 480 - RNA, 19 nt, Artificial Sequence
  Description of Artificial Sequence Synthetic oligonucleotide
caauacgucauuauucuua

SEQ ID NO: 481 - RNA, 19 nt, Artificial Sequence
  Description of Artificial Sequence Synthetic oligonucleotide
aacuaugcaccuauaaaua

SEQ ID NO: 482 - RNA, 19 nt, Artificial Sequence
  Description of Artificial Sequence Synthetic oligonucleotide
uauuuauaggugcauaguu

SEQ ID NO: 483 - RNA, 19 nt, Artificial Sequence
  Description of Artificial Sequence Synthetic oligonucleotide
aaauuuuaccauuuugcga

SEQ ID NO: 484 - RNA, 19 nt, Artificial Sequence
  Description of Artificial Sequence Synthetic oligonucleotide
ucgcaaaaugguaaaauuu

SEQ ID NO: 485 - RNA, 19 nt, Artificial Sequence
  Description of Artificial Sequence Synthetic oligonucleotide
aauuuuaccauuuugcgau

SEQ ID NO: 486 - RNA, 19 nt, Artificial Sequence
  Description of Artificial Sequence Synthetic oligonucleotide
aucgcaaaaugguaaaauu

SEQ ID NO: 487 - RNA, 19 nt, Artificial Sequence
  Description of Artificial Sequence Synthetic oligonucleotide
uuuuaccauuuugcgaugu

SEQ ID NO: 488 - RNA, 19 nt, Artificial Sequence
  Description of Artificial Sequence Synthetic oligonucleotide
acaucgcaaaaugguaaaa

SEQ ID NO: 489 - RNA, 19 nt, Artificial Sequence
  Description of Artificial Sequence Synthetic oligonucleotide
uugcgauguguuuuauuca

SEQ ID NO: 490 - RNA, 19 nt, Artificial Sequence
  Description of Artificial Sequence Synthetic oligonucleotide
ugaauaaaacacaucgcaa

SEQ ID NO: 491 - RNA, 19 nt, Artificial Sequence
  Description of Artificial Sequence Synthetic oligonucleotide
ugcgauguguuuuauucac

SEQ ID NO: 492 - RNA, 19 nt, Artificial Sequence
  Description of Artificial Sequence Synthetic oligonucleotide
gugaauaaaacacaucgca

SEQ ID NO: 493 - RNA, 19 nt, Artificial Sequence
  Description of Artificial Sequence Synthetic oligonucleotide
cguuaucucauugcaaaaa

SEQ ID NO: 494 - RNA, 19 nt, Artificial Sequence
  Description of Artificial Sequence Synthetic oligonucleotide
uuuuugcaaugagauaacg

SEQ ID NO: 495 - RNA, 19 nt, Artificial Sequence
  Description of Artificial Sequence Synthetic oligonucleotide
uaucccaucucacuuuaau

SEQ ID NO: 496 - RNA, 19 nt, Artificial Sequence
  Description of Artificial Sequence Synthetic oligonucleotide
auuaaagugagaugggaua

SEQ ID NO: 497 - RNA, 19 nt, Artificial Sequence
  Description of Artificial Sequence Synthetic oligonucleotide
aucccaucucacuuuaaua

SEQ ID NO: 498 - RNA, 19 nt, Artificial Sequence
  Description of Artificial Sequence Synthetic oligonucleotide
uauuaaagugagaugggau

SEQ ID NO: 499 - RNA, 19 nt, Artificial Sequence
  Description of Artificial Sequence Synthetic oligonucleotide
caucucacuuuaauaauaa

SEQ ID NO: 500 - RNA, 19 nt, Artificial Sequence
  Description of Artificial Sequence Synthetic oligonucleotide
uuauuauuaaagugagaug

SEQ ID NO: 501 - RNA, 19 nt, Artificial Sequence
  Description of Artificial Sequence Synthetic oligonucleotide
aaaucaugcuuauaagcaa

SEQ ID NO: 502 - RNA, 19 nt, Artificial Sequence
  Description of Artificial Sequence Synthetic oligonucleotide
uugcuuauaagcaugauuu

SEQ ID NO: 503 - RNA, 19 nt, Artificial Sequence
  Description of Artificial Sequence Synthetic oligonucleotide
augcuuauaagcaacauga

SEQ ID NO: 504 - RNA, 19 nt, Artificial Sequence
  Description of Artificial Sequence Synthetic oligonucleotide
ucauguugcuuauaagcau

SEQ ID NO: 505 - RNA, 19 nt, Artificial Sequence
  Description of Artificial Sequence Synthetic oligonucleotide
agaacugacacaaaggaca

SEQ ID NO: 506 - RNA, 19 nt, Artificial Sequence
  Description of Artificial Sequence Synthetic oligonucleotide
uguccuuugugucaguucu

SEQ ID NO: 507 - RNA, 19 nt, Artificial Sequence
  Description of Artificial Sequence Synthetic oligonucleotide
auaaaguuauuaauagcca

SEQ ID NO: 508 - RNA, 19 nt, Artificial Sequence
  Description of Artificial Sequence Synthetic oligonucleotide
uggcuauuaauaacuuuau

SEQ ID NO: 509 - RNA, 19 nt, Artificial Sequence
  Description of Artificial Sequence Synthetic oligonucleotide
aaaguuauuaauagccauu

SEQ ID NO: 510 - RNA, 19 nt, Artificial Sequence
  Description of Artificial Sequence Synthetic oligonucleotide
aauggcuauuaauaacuuu

SEQ ID NO: 511 - RNA, 19 nt, Artificial Sequence
  Description of Artificial Sequence Synthetic oligonucleotide
ggauguauucaugaaagga

SEQ ID NO: 512 - RNA, 19 nt, Artificial Sequence
  Description of Artificial Sequence Synthetic oligonucleotide
uccuuucaugaauacaucc

SEQ ID NO: 513 - RNA, 19 nt, Artificial Sequence
  Description of Artificial Sequence Synthetic oligonucleotide
guucucuauguaggcucca

SEQ ID NO: 514 - RNA, 19 nt, Artificial Sequence
  Description of Artificial Sequence Synthetic oligonucleotide
uggagccuacauagagaac

SEQ ID NO: 515 - RNA, 19 nt, Artificial Sequence
  Description of Artificial Sequence Synthetic oligonucleotide
guuccauccuguacaagug

SEQ ID NO: 516 - RNA, 19 nt, Artificial Sequence
  Description of Artificial Sequence Synthetic oligonucleotide
cacuuguacaggauggaac

SEQ ID NO: 517 - RNA, 19 nt, Artificial Sequence
  Description of Artificial Sequence Synthetic oligonucleotide
cuaaauccucacuauuuuu

SEQ ID NO: 518 - RNA, 19 nt, Artificial Sequence
  Description of Artificial Sequence Synthetic oligonucleotide
aaaaauagugaggauuuag

SEQ ID NO: 519 - RNA, 19 nt, Artificial Sequence
  Description of Artificial Sequence Synthetic oligonucleotide
cuauguaggcuccaaaacc

SEQ ID NO: 520 - RNA, 19 nt, Artificial Sequence
  Description of Artificial Sequence Synthetic oligonucleotide
gguuuuggagccuacauag

SEQ ID NO: 521 - RNA, 19 nt, Artificial Sequence
  Description of Artificial Sequence Synthetic oligonucleotide
ggugcaugguguggcaaca

SEQ ID NO: 522 - RNA, 19 nt, Artificial Sequence
  Description of Artificial Sequence Synthetic oligonucleotide
uguugccacaccaugcacc

SEQ ID NO: 523 - RNA, 19 nt, Artificial Sequence
  Description of Artificial Sequence Synthetic oligonucleotide
ugagaucugcugacagaug

SEQ ID NO: 524 - RNA, 19 nt, Artificial Sequence
  Description of Artificial Sequence Synthetic oligonucleotide
caucugucagcagaucuca

SEQ ID NO: 525 - RNA, 19 nt, Artificial Sequence
  Description of Artificial Sequence Synthetic oligonucleotide
cccacucagcauuucggug

SEQ ID NO: 526 - RNA, 19 nt, Artificial Sequence
  Description of Artificial Sequence Synthetic oligonucleotide
caccgaaaugcugaguggg

SEQ ID NO: 527 - RNA, 19 nt, Artificial Sequence
  Description of Artificial Sequence Synthetic oligonucleotide
caugguagcagggucuuug

SEQ ID NO: 528 - RNA, 19 nt, Artificial Sequence
  Description of Artificial Sequence Synthetic oligonucleotide
caaagacccugcuaccaug

SEQ ID NO: 529 - RNA, 19 nt, Artificial Sequence
  Description of Artificial Sequence Synthetic oligonucleotide
agaauuaaaauaaaacguu

SEQ ID NO: 530 - RNA, 19 nt, Artificial Sequence
  Description of Artificial Sequence Synthetic oligonucleotide
aacguuuuauuuuaauucu

SEQ ID NO: 531 - RNA, 19 nt, Artificial Sequence
  Description of Artificial Sequence Synthetic oligonucleotide
aucucgaagucuuccauca

SEQ ID NO: 532 - RNA, 19 nt, Artificial Sequence
  Description of Artificial Sequence Synthetic oligonucleotide
ugauggaagacuucgagau

SEQ ID NO: 533 - RNA, 19 nt, Artificial Sequence
  Description of Artificial Sequence Synthetic oligonucleotide
gugagcaugaaacuaugca

SEQ ID NO: 534 - RNA, 19 nt, Artificial Sequence
  Description of Artificial Sequence Synthetic oligonucleotide
ugcauaguuucaugcucac

SEQ ID NO: 535 - DNA, 21 bp, Artificial Sequence
  Description of Artificial Sequence Synthetic oligonucleotide
guggcuucaaucuacgaugnn

SEQ ID NO: 536 - DNA, 21 bp, Artificial Sequence
  Description of Artificial Sequence Synthetic oligonucleotide
caucguagauugaagccacnn

SEQ ID NO: 537 - DNA, 21 bp, Artificial Sequence
  Description of Artificial Sequence Synthetic oligonucleotide
uuuuuacaguguaucucgann

SEQ ID NO: 538 - DNA, 21 bp, Artificial Sequence
  Description of Artificial Sequence Synthetic oligonucleotide
ucgagauacacuguaaaaann

SEQ ID NO: 539 - DNA, 21 bp, Artificial Sequence
  Description of Artificial Sequence Synthetic oligonucleotide
guaucaagacuacgaaccunn

SEQ ID NO: 540 - DNA, 21 bp, Artificial Sequence
  Description of Artificial Sequence Synthetic oligonucleotide
agguucguagucuugauacnn

SEQ ID NO: 541 - DNA, 21 bp, Artificial Sequence
  Description of Artificial Sequence Synthetic oligonucleotide
uuuuacaguguaucucgaann

SEQ ID NO: 542 - DNA, 21 bp, Artificial Sequence
  Description of Artificial Sequence Synthetic oligonucleotide
uucgagauacacuguaaaann

SEQ ID NO: 543 - DNA, 21 bp, Artificial Sequence
  Description of Artificial Sequence Synthetic oligonucleotide
acauuaacccuacacucggnn

SEQ ID NO: 544 - DNA, 21 bp, Artificial Sequence
  Description of Artificial Sequence Synthetic oligonucleotide
ccgaguguaggguuaaugunn

SEQ ID NO: 545 - DNA, 21 bp, Artificial Sequence
  Description of Artificial Sequence Synthetic oligonucleotide
uguggcuucaaucuacgaunn

SEQ ID NO: 546 - DNA, 21 bp, Artificial Sequence
  Description of Artificial Sequence Synthetic oligonucleotide
aucguagauugaagccacann

SEQ ID NO: 547 - DNA, 21 bp, Artificial Sequence
  Description of Artificial Sequence Synthetic oligonucleotide
cucagcauuucggugcuucnn

SEQ ID NO: 548 - DNA, 21 bp, Artificial Sequence
  Description of Artificial Sequence Synthetic oligonucleotide
gaagcaccgaaaugcugagnn

SEQ ID NO: 549 - DNA, 21 bp, Artificial Sequence
  Description of Artificial Sequence Synthetic oligonucleotide
uuaacccuacacucggaaunn

SEQ ID NO: 550 - DNA, 21 bp, Artificial Sequence
  Description of Artificial Sequence Synthetic oligonucleotide
auuccgaguguaggguuaann

SEQ ID NO: 551 - DNA, 21 bp, Artificial Sequence
  Description of Artificial Sequence Synthetic oligonucleotide
uuacaguguaucucgaagunn

SEQ ID NO: 552 - DNA, 21 bp, Artificial Sequence
  Description of Artificial Sequence Synthetic oligonucleotide
acuucgagauacacuguaann

SEQ ID NO: 553 - DNA, 21 bp, Artificial Sequence
  Description of Artificial Sequence Synthetic oligonucleotide
acaguguaucucgaagucunn

SEQ ID NO: 554 - DNA, 21 bp, Artificial Sequence
  Description of Artificial Sequence Synthetic oligonucleotide
agacuucgagauacacugunn

SEQ ID NO: 555 - DNA, 21 bp, Artificial Sequence
  Description of Artificial Sequence Synthetic oligonucleotide
gcuucaaucuacgauguuann

SEQ ID NO: 556 - DNA, 21 bp, Artificial Sequence
  Description of Artificial Sequence Synthetic oligonucleotide
uaacaucguagauugaagcnn

SEQ ID NO: 557 - DNA, 21 bp, Artificial Sequence
  Description of Artificial Sequence Synthetic oligonucleotide
auuaacccuacacucggaann

SEQ ID NO: 558 - DNA, 21 bp, Artificial Sequence
  Description of Artificial Sequence Synthetic oligonucleotide
uuccgaguguaggguuaaunn

SEQ ID NO: 559 - DNA, 21 bp, Artificial Sequence
  Description of Artificial Sequence Synthetic oligonucleotide
uuaaugauacugucuaagann

SEQ ID NO: 560 - DNA, 21 bp, Artificial Sequence
  Description of Artificial Sequence Synthetic oligonucleotide
ucuuagacaguaucauuaann

SEQ ID NO: 561 - DNA, 21 bp, Artificial Sequence
  Description of Artificial Sequence Synthetic oligonucleotide
aucaagacuacgaaccugann

SEQ ID NO: 562 - DNA, 21 bp, Artificial Sequence
  Description of Artificial Sequence Synthetic oligonucleotide
ucagguucguagucuugaunn

SEQ ID NO: 563 - DNA, 21 bp, Artificial Sequence
  Description of Artificial Sequence Synthetic oligonucleotide
agugaauacaugguagcagnn

SEQ ID NO: 564 - DNA, 21 bp, Artificial Sequence
  Description of Artificial Sequence Synthetic oligonucleotide
cugcuaccauguauucacunn

SEQ ID NO: 565 - DNA, 21 bp, Artificial Sequence
  Description of Artificial Sequence Synthetic oligonucleotide
aauacaugguagcagggucnn

SEQ ID NO: 566 - DNA, 21 bp, Artificial Sequence
  Description of Artificial Sequence Synthetic oligonucleotide
gacccugcuaccauguauunn

SEQ ID NO: 567 - DNA, 21 bp, Artificial Sequence
  Description of Artificial Sequence Synthetic oligonucleotide
cuaagugacuaccacuuaunn

SEQ ID NO: 568 - DNA, 21 bp, Artificial Sequence
  Description of Artificial Sequence Synthetic oligonucleotide
auaagugguagucacuuagnn

SEQ ID NO: 569 - DNA, 21 bp, Artificial Sequence
  Description of Artificial Sequence Synthetic oligonucleotide
agcaugaaacuaugcaccunn

SEQ ID NO: 570 - DNA, 21 bp, Artificial Sequence
  Description of Artificial Sequence Synthetic oligonucleotide
aggugcauaguuucaugcunn

SEQ ID NO: 571 - DNA, 21 bp, Artificial Sequence
  Description of Artificial Sequence Synthetic oligonucleotide
uuguggcuucaaucuacgann

SEQ ID NO: 572 - DNA, 21 bp, Artificial Sequence
  Description of Artificial Sequence Synthetic oligonucleotide
ucguagauugaagccacaann

SEQ ID NO: 573 - DNA, 21 bp, Artificial Sequence
  Description of Artificial Sequence Synthetic oligonucleotide
ggcuucaaucuacgauguunn

SEQ ID NO: 574 - DNA, 21 bp, Artificial Sequence
  Description of Artificial Sequence Synthetic oligonucleotide
aacaucguagauugaagccnn

SEQ ID NO: 575 - DNA, 21 bp, Artificial Sequence
  Description of Artificial Sequence Synthetic oligonucleotide
auaaugacguauugugaaann

SEQ ID NO: 576 - DNA, 21 bp, Artificial Sequence
  Description of Artificial Sequence Synthetic oligonucleotide
uuucacaauacgucauuaunn

SEQ ID NO: 577 - DNA, 21 bp, Artificial Sequence
  Description of Artificial Sequence Synthetic oligonucleotide
agugauuugcuaucauauann

SEQ ID NO: 578 - DNA, 21 bp, Artificial Sequence
  Description of Artificial Sequence Synthetic oligonucleotide
uauaugauagcaaaucacunn

SEQ ID NO: 579 - DNA, 21 bp, Artificial Sequence
  Description of Artificial Sequence Synthetic oligonucleotide
caaucuacgauguuaaaacnn

SEQ ID NO: 580 - DNA, 21 bp, Artificial Sequence
  Description of Artificial Sequence Synthetic oligonucleotide
guuuuaacaucguagauugnn

SEQ ID NO: 581 - DNA, 21 bp, Artificial Sequence
  Description of Artificial Sequence Synthetic oligonucleotide
acguauugugaaauuuguunn

SEQ ID NO: 582 - DNA, 21 bp, Artificial Sequence
  Description of Artificial Sequence Synthetic oligonucleotide
aacaaauuucacaauacgunn

SEQ ID NO: 583 - DNA, 21 bp, Artificial Sequence
  Description of Artificial Sequence Synthetic oligonucleotide
gacguauugugaaauuugunn

SEQ ID NO: 584 - DNA, 21 bp, Artificial Sequence
  Description of Artificial Sequence Synthetic oligonucleotide
acaaauuucacaauacgucnn

SEQ ID NO: 585 - DNA, 21 bp, Artificial Sequence
  Description of Artificial Sequence Synthetic oligonucleotide
acgggugugacagcaguagnn

SEQ ID NO: 586 - DNA, 21 bp, Artificial Sequence
  Description of Artificial Sequence Synthetic oligonucleotide
cuacugcugucacacccgunn

SEQ ID NO: 587 - DNA, 21 bp, Artificial Sequence
  Description of Artificial Sequence Synthetic oligonucleotide
gguaucaagacuacgaaccnn

SEQ ID NO: 588 - DNA, 21 bp, Artificial Sequence
  Description of Artificial Sequence Synthetic oligonucleotide
gguucguagucuugauaccnn

SEQ ID NO: 589 - DNA, 21 bp, Artificial Sequence
  Description of Artificial Sequence Synthetic oligonucleotide
ugucuaagaauaaugacgunn

SEQ ID NO: 590 - DNA, 21 bp, Artificial Sequence
  Description of Artificial Sequence Synthetic oligonucleotide
acgucauuauucuuagacann

SEQ ID NO: 591 - DNA, 21 bp, Artificial Sequence
  Description of Artificial Sequence Synthetic oligonucleotide
aacccuacacucggaauucnn

SEQ ID NO: 592 - DNA, 21 bp, Artificial Sequence
  Description of Artificial Sequence Synthetic oligonucleotide
gaauuccgaguguaggguunn

SEQ ID NO: 593 - DNA, 21 bp, Artificial Sequence
  Description of Artificial Sequence Synthetic oligonucleotide
aaaagaggguguucucuaunn

SEQ ID NO: 594 - DNA, 21 bp, Artificial Sequence
  Description of Artificial Sequence Synthetic oligonucleotide
auagagaacacccucuuuunn

SEQ ID NO: 595 - DNA, 21 bp, Artificial Sequence
  Description of Artificial Sequence Synthetic oligonucleotide
ggguguucucuauguaggcnn

SEQ ID NO: 596 - DNA, 21 bp, Artificial Sequence
  Description of Artificial Sequence Synthetic oligonucleotide
gccuacauagagaacacccnn

SEQ ID NO: 597 - DNA, 21 bp, Artificial Sequence
  Description of Artificial Sequence Synthetic oligonucleotide
ucucuauguaggcuccaaann

SEQ ID NO: 598 - DNA, 21 bp, Artificial Sequence
  Description of Artificial Sequence Synthetic oligonucleotide
uuuggagccuacauagagann

SEQ ID NO: 599 - DNA, 21 bp, Artificial Sequence
  Description of Artificial Sequence Synthetic oligonucleotide
gaaggguaucaagacuacgnn

SEQ ID NO: 600 - DNA, 21 bp, Artificial Sequence
  Description of Artificial Sequence Synthetic oligonucleotide
cguagucuugauacccuucnn

SEQ ID NO: 601 - DNA, 21 bp, Artificial Sequence
  Description of Artificial Sequence Synthetic oligonucleotide
aaggguaucaagacuacgann

SEQ ID NO: 602 - DNA, 21 bp, Artificial Sequence
  Description of Artificial Sequence Synthetic oligonucleotide
ucguagucuugauacccuunn

SEQ ID NO: 603 - DNA, 21 bp, Artificial Sequence
  Description of Artificial Sequence Synthetic oligonucleotide
ggguaucaagacuacgaacnn

SEQ ID NO: 604 - DNA, 21 bp, Artificial Sequence
  Description of Artificial Sequence Synthetic oligonucleotide
guucguagucuugauacccnn

SEQ ID NO: 605 - DNA, 21 bp, Artificial Sequence
  Description of Artificial Sequence Synthetic oligonucleotide
gaagccuaagaaauaucuunn

SEQ ID NO: 606 - DNA, 21 bp, Artificial Sequence
  Description of Artificial Sequence Synthetic oligonucleotide
aagauauuucuuaggcuucnn

SEQ ID NO: 607 - DNA, 21 bp, Artificial Sequence
  Description of Artificial Sequence Synthetic oligonucleotide
uuuacaguguaucucgaagnn

SEQ ID NO: 608 - DNA, 21 bp, Artificial Sequence
  Description of Artificial Sequence Synthetic oligonucleotide
cuucgagauacacuguaaann

SEQ ID NO: 609 - DNA, 21 bp, Artificial Sequence
  Description of Artificial Sequence Synthetic oligonucleotide
uacaguguaucucgaagucnn

SEQ ID NO: 610 - DNA, 21 bp, Artificial Sequence
  Description of Artificial Sequence Synthetic oligonucleotide
gacuucgagauacacuguann

SEQ ID NO: 611 - DNA, 21 bp, Artificial Sequence
  Description of Artificial Sequence Synthetic oligonucleotide
cacucagcauuucggugcunn

SEQ ID NO: 612 - DNA, 21 bp, Artificial Sequence
  Description of Artificial Sequence Synthetic oligonucleotide
agcaccgaaaugcugagugnn

SEQ ID NO: 613 - DNA, 21 bp, Artificial Sequence
  Description of Artificial Sequence Synthetic oligonucleotide
uggcuucaaucuacgaugunn

SEQ ID NO: 614 - DNA, 21 bp, Artificial Sequence
  Description of Artificial Sequence Synthetic oligonucleotide
acaucguagauugaagccann

SEQ ID NO: 615 - DNA, 21 bp, Artificial Sequence
  Description of Artificial Sequence Synthetic oligonucleotide
cuucaaucuacgauguuaann

SEQ ID NO: 616 - DNA, 21 bp, Artificial Sequence
  Description of Artificial Sequence Synthetic oligonucleotide
uuaacaucguagauugaagnn

SEQ ID NO: 617 - DNA, 21 bp, Artificial Sequence
  Description of Artificial Sequence Synthetic oligonucleotide
aaucuacgauguuaaaacann

SEQ ID NO: 618 - DNA, 21 bp, Artificial Sequence
  Description of Artificial Sequence Synthetic oligonucleotide
uguuuuaacaucguagauunn

SEQ ID NO: 619 - DNA, 21 bp, Artificial Sequence
  Description of Artificial Sequence Synthetic oligonucleotide
aacaccuaagugacuaccann

SEQ ID NO: 620 - DNA, 21 bp, Artificial Sequence
  Description of Artificial Sequence Synthetic oligonucleotide
ugguagucacuuagguguunn

SEQ ID NO: 621 - DNA, 21 bp, Artificial Sequence
  Description of Artificial Sequence Synthetic oligonucleotide
uaagugacuaccacuuauunn

SEQ ID NO: 622 - DNA, 21 bp, Artificial Sequence
  Description of Artificial Sequence Synthetic oligonucleotide
aauaagugguagucacuuann

SEQ ID NO: 623 - DNA, 21 bp, Artificial Sequence
  Description of Artificial Sequence Synthetic oligonucleotide
uguucagaaguuguuagugnn

SEQ ID NO: 624 - DNA, 21 bp, Artificial Sequence
  Description of Artificial Sequence Synthetic oligonucleotide
cacuaacaacuucugaacann

SEQ ID NO: 625 - DNA, 21 bp, Artificial Sequence
  Description of Artificial Sequence Synthetic oligonucleotide
aaguuguuagugauuugcunn

SEQ ID NO: 626 - DNA, 21 bp, Artificial Sequence
  Description of Artificial Sequence Synthetic oligonucleotide
agcaaaucacuaacaacuunn

SEQ ID NO: 627 - DNA, 21 bp, Artificial Sequence
  Description of Artificial Sequence Synthetic oligonucleotide
auaagauuuuuaggugucunn

SEQ ID NO: 628 - DNA, 21 bp, Artificial Sequence
  Description of Artificial Sequence Synthetic oligonucleotide
agacaccuaaaaaucuuaunn

SEQ ID NO: 629 - DNA, 21 bp, Artificial Sequence
  Description of Artificial Sequence Synthetic oligonucleotide
aaugacguauugugaaauunn

SEQ ID NO: 630 - DNA, 21 bp, Artificial Sequence
  Description of Artificial Sequence Synthetic oligonucleotide
aauuucacaauacgucauunn

SEQ ID NO: 631 - DNA, 21 bp, Artificial Sequence
  Description of Artificial Sequence Synthetic oligonucleotide
gcaugaaacuaugcaccuann

SEQ ID NO: 632 - DNA, 21 bp, Artificial Sequence
  Description of Artificial Sequence Synthetic oligonucleotide
uaggugcauaguuucaugcnn

SEQ ID NO: 633 - DNA, 21 bp, Artificial Sequence
  Description of Artificial Sequence Synthetic oligonucleotide
uaugcaccuauaaauacuann

SEQ ID NO: 634 - DNA, 21 bp, Artificial Sequence
  Description of Artificial Sequence Synthetic oligonucleotide
uaguauuuauaggugcauann

SEQ ID NO: 635 - DNA, 21 bp, Artificial Sequence
  Description of Artificial Sequence Synthetic oligonucleotide
uaccauuuugcgauguguunn

SEQ ID NO: 636 - DNA, 21 bp, Artificial Sequence
  Description of Artificial Sequence Synthetic oligonucleotide
aacacaucgcaaaaugguann

SEQ ID NO: 637 - DNA, 21 bp, Artificial Sequence
  Description of Artificial Sequence Synthetic oligonucleotide
auuuuuaucccaucucacunn

SEQ ID NO: 638 - DNA, 21 bp, Artificial Sequence
  Description of Artificial Sequence Synthetic oligonucleotide
agugagaugggauaaaaaunn

SEQ ID NO: 639 - DNA, 21 bp, Artificial Sequence
  Description of Artificial Sequence Synthetic oligonucleotide
uuaucccaucucacuuuaann

SEQ ID NO: 640 - DNA, 21 bp, Artificial Sequence
  Description of Artificial Sequence Synthetic oligonucleotide
uuaaagugagaugggauaann

SEQ ID NO: 641 - DNA, 21 bp, Artificial Sequence
  Description of Artificial Sequence Synthetic oligonucleotide
uaacccuacacucggaauunn

SEQ ID NO: 642 - DNA, 21 bp, Artificial Sequence
  Description of Artificial Sequence Synthetic oligonucleotide
aauuccgaguguaggguuann

SEQ ID NO: 643 - DNA, 21 bp, Artificial Sequence
  Description of Artificial Sequence Synthetic oligonucleotide
aggguguucucuauguaggnn

SEQ ID NO: 644 - DNA, 21 bp, Artificial Sequence
  Description of Artificial Sequence Synthetic oligonucleotide
ccuacauagagaacacccunn

SEQ ID NO: 645 - DNA, 21 bp, Artificial Sequence
  Description of Artificial Sequence Synthetic oligonucleotide
cucuauguaggcuccaaaann

SEQ ID NO: 646 - DNA, 21 bp, Artificial Sequence
  Description of Artificial Sequence Synthetic oligonucleotide
uuuuggagccuacauagagnn

SEQ ID NO: 647 - DNA, 21 bp, Artificial Sequence
  Description of Artificial Sequence Synthetic oligonucleotide
aaccaaacaggguguggcann

SEQ ID NO: 648 - DNA, 21 bp, Artificial Sequence
  Description of Artificial Sequence Synthetic oligonucleotide
ugccacacccuguuugguunn

SEQ ID NO: 649 - DNA, 21 bp, Artificial Sequence
  Description of Artificial Sequence Synthetic oligonucleotide
gaggguguucucuauguagnn

SEQ ID NO: 650 - DNA, 21 bp, Artificial Sequence
  Description of Artificial Sequence Synthetic oligonucleotide
cuacauagagaacacccucnn

SEQ ID NO: 651 - DNA, 21 bp, Artificial Sequence
  Description of Artificial Sequence Synthetic oligonucleotide
uucucuauguaggcuccaann

SEQ ID NO: 652 - DNA, 21 bp, Artificial Sequence
  Description of Artificial Sequence Synthetic oligonucleotide
uuggagccuacauagagaann

SEQ ID NO: 653 - DNA, 21 bp, Artificial Sequence
  Description of Artificial Sequence Synthetic oligonucleotide
gugacaaauguuggaggagnn

SEQ ID NO: 654 - DNA, 21 bp, Artificial Sequence
  Description of Artificial Sequence Synthetic oligonucleotide
cuccuccaacauuugucacnn

SEQ ID NO: 655 - DNA, 21 bp, Artificial Sequence
  Description of Artificial Sequence Synthetic oligonucleotide
gucaaaaaggaccaguuggnn

SEQ ID NO: 656 - DNA, 21 bp, Artificial Sequence
  Description of Artificial Sequence Synthetic oligonucleotide
ccaacugguccuuuuugacnn

SEQ ID NO: 657 - DNA, 21 bp, Artificial Sequence
  Description of Artificial Sequence Synthetic oligonucleotide
ucaagacuacgaaccugaann

SEQ ID NO: 658 - DNA, 21 bp, Artificial Sequence
  Description of Artificial Sequence Synthetic oligonucleotide
uucagguucguagucuugann

SEQ ID NO: 659 - DNA, 21 bp, Artificial Sequence
  Description of Artificial Sequence Synthetic oligonucleotide
cagcauuucggugcuucccnn

SEQ ID NO: 660 - DNA, 21 bp, Artificial Sequence
  Description of Artificial Sequence Synthetic oligonucleotide
gggaagcaccgaaaugcugnn

SEQ ID NO: 661 - DNA, 21 bp, Artificial Sequence
  Description of Artificial Sequence Synthetic oligonucleotide
guuguucagaaguuguuagnn

SEQ ID NO: 662 - DNA, 21 bp, Artificial Sequence
  Description of Artificial Sequence Synthetic oligonucleotide
cuaacaacuucugaacaacnn

SEQ ID NO: 663 - DNA, 21 bp, Artificial Sequence
  Description of Artificial Sequence Synthetic oligonucleotide
gaaguuguuagugauuugcnn

SEQ ID NO: 664 - DNA, 21 bp, Artificial Sequence
  Description of Artificial Sequence Synthetic oligonucleotide
gcaaaucacuaacaacuucnn

SEQ ID NO: 665 - DNA, 21 bp, Artificial Sequence
  Description of Artificial Sequence Synthetic oligonucleotide
aguuguuagugauuugcuann

SEQ ID NO: 666 - DNA, 21 bp, Artificial Sequence
  Description of Artificial Sequence Synthetic oligonucleotide
uagcaaaucacuaacaacunn

SEQ ID NO: 667 - DNA, 21 bp, Artificial Sequence
  Description of Artificial Sequence Synthetic oligonucleotide
uuagugauuugcuaucauann

SEQ ID NO: 668 - DNA, 21 bp, Artificial Sequence
  Description of Artificial Sequence Synthetic oligonucleotide
uaugauagcaaaucacuaann

SEQ ID NO: 669 - DNA, 21 bp, Artificial Sequence
  Description of Artificial Sequence Synthetic oligonucleotide
gugauuugcuaucauauaunn

SEQ ID NO: 670 - DNA, 21 bp, Artificial Sequence
  Description of Artificial Sequence Synthetic oligonucleotide
auauaugauagcaaaucacnn

SEQ ID NO: 671 - DNA, 21 bp, Artificial Sequence
  Description of Artificial Sequence Synthetic oligonucleotide
uaaugacguauugugaaaunn

SEQ ID NO: 672 - DNA, 21 bp, Artificial Sequence
  Description of Artificial Sequence Synthetic oligonucleotide
auuucacaauacgucauuann

SEQ ID NO: 673 - DNA, 21 bp, Artificial Sequence
  Description of Artificial Sequence Synthetic oligonucleotide
cuaugcaccuauaaauacunn

SEQ ID NO: 674 - DNA, 21 bp, Artificial Sequence
  Description of Artificial Sequence Synthetic oligonucleotide
aguauuuauaggugcauagnn

SEQ ID NO: 675 - DNA, 21 bp, Artificial Sequence
  Description of Artificial Sequence Synthetic oligonucleotide
auuagccauggauguauucnn

SEQ ID NO: 676 - DNA, 21 bp, Artificial Sequence
  Description of Artificial Sequence Synthetic oligonucleotide
gaauacauccauggcuaaunn

SEQ ID NO: 677 - DNA, 21 bp, Artificial Sequence
  Description of Artificial Sequence Synthetic oligonucleotide
uuagccauggauguauucann

SEQ ID NO: 678 - DNA, 21 bp, Artificial Sequence
  Description of Artificial Sequence Synthetic oligonucleotide
ugaauacauccauggcuaann

SEQ ID NO: 679 - DNA, 21 bp, Artificial Sequence
  Description of Artificial Sequence Synthetic oligonucleotide
gaaaggacuuucaaaggccnn

SEQ ID NO: 680 - DNA, 21 bp, Artificial Sequence
  Description of Artificial Sequence Synthetic oligonucleotide
ggccuuugaaaguccuuucnn

SEQ ID NO: 681 - DNA, 21 bp, Artificial Sequence
  Description of Artificial Sequence Synthetic oligonucleotide
caaaagaggguguucucuann

SEQ ID NO: 682 - DNA, 21 bp, Artificial Sequence
  Description of Artificial Sequence Synthetic oligonucleotide
uagagaacacccucuuuugnn

SEQ ID NO: 683 - DNA, 21 bp, Artificial Sequence
  Description of Artificial Sequence Synthetic oligonucleotide
agaggguguucucuauguann

SEQ ID NO: 684 - DNA, 21 bp, Artificial Sequence
  Description of Artificial Sequence Synthetic oligonucleotide
uacauagagaacacccucunn

SEQ ID NO: 685 - DNA, 21 bp, Artificial Sequence
  Description of Artificial Sequence Synthetic oligonucleotide
gguguucucuauguaggcunn

SEQ ID NO: 686 - DNA, 21 bp, Artificial Sequence
  Description of Combined DNA/RNA Molecule Synthetic oligonucleotide
agccuacauagagaacaccnn

SEQ ID NO: 687 - DNA, 21 bp, Artificial Sequence
  Description of Artificial Sequence Synthetic oligonucleotide
guguucucuauguaggcucnn

SEQ ID NO: 688 - DNA, 21 bp, Artificial Sequence
  Description of Artificial Sequence Synthetic oligonucleotide
gagccuacauagagaacacnn

SEQ ID NO: 689 - DNA, 21 bp, Artificial Sequence
  Description of Artificial Sequence Synthetic oligonucleotide
uguucucuauguaggcuccnn

SEQ ID NO: 690 - DNA, 21 bp, Artificial Sequence
  Description of Artificial Sequence Synthetic oligonucleotide
ggagccuacauagagaacann

SEQ ID NO: 691 - DNA, 21 bp, Artificial Sequence
  Description of Artificial Sequence Synthetic oligonucleotide
ggagcaguggugacgggugnn

SEQ ID NO: 692 - DNA, 21 bp, Artificial Sequence
  Description of Artificial Sequence Synthetic oligonucleotide
cacccgucaccacugcuccnn

SEQ ID NO: 693 - DNA, 21 bp, Artificial Sequence
  Description of Artificial Sequence Synthetic oligonucleotide
caguggugacgggugugacnn

SEQ ID NO: 694 - DNA, 21 bp, Artificial Sequence
  Description of Artificial Sequence Synthetic oligonucleotide
gucacacccgucaccacugnn

SEQ ID NO: 695 - DNA, 21 bp, Artificial Sequence
  Description of Artificial Sequence Synthetic oligonucleotide
uaucaagacuacgaaccugnn

SEQ ID NO: 696 - DNA, 21 bp, Artificial Sequence
  Description of Artificial Sequence Synthetic oligonucleotide
cagguucguagucuugauann

SEQ ID NO: 697 - DNA, 21 bp, Artificial Sequence
  Description of Artificial Sequence Synthetic oligonucleotide
cucccaguuucuugagaucnn

SEQ ID NO: 698 - DNA, 21 bp, Artificial Sequence
  Description of Artificial Sequence Synthetic oligonucleotide
gaucucaagaaacugggagnn

SEQ ID NO: 699 - DNA, 21 bp, Artificial Sequence
  Description of Artificial Sequence Synthetic oligonucleotide
gacagauguuccauccugunn

SEQ ID NO: 700 - DNA, 21 bp, Artificial Sequence
  Description of Artificial Sequence Synthetic oligonucleotide
acaggauggaacaucugucnn

SEQ ID NO: 701 - DNA, 21 bp, Artificial Sequence
  Description of Artificial Sequence Synthetic oligonucleotide
aaugugcccagucaugacann

SEQ ID NO: 702 - DNA, 21 bp, Artificial Sequence
  Description of Artificial Sequence Synthetic oligonucleotide
ugucaugacugggcacauunn

SEQ ID NO: 703 - DNA, 21 bp, Artificial Sequence
  Description of Artificial Sequence Synthetic oligonucleotide
ugcccagucaugacauuucnn

SEQ ID NO: 704 - DNA, 21 bp, Artificial Sequence
  Description of Artificial Sequence Synthetic oligonucleotide
gaaaugucaugacugggcann

SEQ ID NO: 705 - DNA, 21 bp, Artificial Sequence
  Description of Artificial Sequence Synthetic oligonucleotide
cagucaugacauuucucaann

SEQ ID NO: 706 - DNA, 21 bp, Artificial Sequence
  Description of Artificial Sequence Synthetic oligonucleotide
uugagaaaugucaugacugnn

SEQ ID NO: 707 - DNA, 21 bp, Artificial Sequence
  Description of Artificial Sequence Synthetic oligonucleotide
aaguuuuuacaguguaucunn

SEQ ID NO: 708 - DNA, 21 bp, Artificial Sequence
  Description of Artificial Sequence Synthetic oligonucleotide
agauacacuguaaaaacuunn

SEQ ID NO: 709 - DNA, 21 bp, Artificial Sequence
  Description of Artificial Sequence Synthetic oligonucleotide
caguguaucucgaagucuunn

SEQ ID NO: 710 - DNA, 21 bp, Artificial Sequence
  Description of Artificial Sequence Synthetic oligonucleotide
aagacuucgagauacacugnn

SEQ ID NO: 711 - DNA, 21 bp, Artificial Sequence
  Description of Artificial Sequence Synthetic oligonucleotide
guaucucgaagucuuccaunn

SEQ ID NO: 712 - DNA, 21 bp, Artificial Sequence
  Description of Artificial Sequence Synthetic oligonucleotide
auggaagacuucgagauacnn

SEQ ID NO: 713 - DNA, 21 bp, Artificial Sequence
  Description of Artificial Sequence Synthetic oligonucleotide
cagcagugauugaaguaucnn

SEQ ID NO: 714 - DNA, 21 bp, Artificial Sequence
  Description of Artificial Sequence Synthetic oligonucleotide
gauacuucaaucacugcugnn

SEQ ID NO: 715 - DNA, 21 bp, Artificial Sequence
  Description of Artificial Sequence Synthetic oligonucleotide
ccacucagcauuucggugcnn

SEQ ID NO: 716 - DNA, 21 bp, Artificial Sequence
  Description of Artificial Sequence Synthetic oligonucleotide
gcaccgaaaugcugaguggnn

SEQ ID NO: 717 - DNA, 21 bp, Artificial Sequence
  Description of Artificial Sequence Synthetic oligonucleotide
ucagcauuucggugcuuccnn

SEQ ID NO: 718 - DNA, 21 bp, Artificial Sequence
  Description of Artificial Sequence Synthetic oligonucleotide
ggaagcaccgaaaugcugann

SEQ ID NO: 719 - DNA, 21 bp, Artificial Sequence
  Description of Artificial Sequence Synthetic oligonucleotide
aagugaauacaugguagcann

SEQ ID NO: 720 - DNA, 21 bp, Artificial Sequence
  Description of Artificial Sequence Synthetic oligonucleotide
ugcuaccauguauucacuunn

SEQ ID NO: 721 - DNA, 21 bp, Artificial Sequence
  Description of Artificial Sequence Synthetic oligonucleotide
gaauacaugguagcagggunn

SEQ ID NO: 722 - DNA, 21 bp, Artificial Sequence
  Description of Artificial Sequence Synthetic oligonucleotide
acccugcuaccauguauucnn

SEQ ID NO: 723 - DNA, 21 bp, Artificial Sequence
  Description of Artificial Sequence Synthetic oligonucleotide
ugguagcagggucuuugugnn

SEQ ID NO: 724 - DNA, 21 bp, Artificial Sequence
  Description of Artificial Sequence Synthetic oligonucleotide
cacaaagacccugcuaccann

SEQ ID NO: 725 - DNA, 21 bp, Artificial Sequence
  Description of Artificial Sequence Synthetic oligonucleotide
uuuuguggcuucaaucuacnn

SEQ ID NO: 726 - DNA, 21 bp, Artificial Sequence
  Description of Artificial Sequence Synthetic oligonucleotide
guagauugaagccacaaaann

SEQ ID NO: 727 - DNA, 21 bp, Artificial Sequence
  Description of Artificial Sequence Synthetic oligonucleotide
uuuguggcuucaaucuacgnn

SEQ ID NO: 728 - DNA, 21 bp, Artificial Sequence
  Description of Artificial Sequence Synthetic oligonucleotide
cguagauugaagccacaaann

SEQ ID NO: 729 - DNA, 21 bp, Artificial Sequence
  Description of Artificial Sequence Synthetic oligonucleotide
aauuaaaaacaccuaagugnn

SEQ ID NO: 730 - DNA, 21 bp, Artificial Sequence
  Description of Artificial Sequence Synthetic oligonucleotide
cacuuagguguuuuuaauunn

SEQ ID NO: 731 - DNA, 21 bp, Artificial Sequence
  Description of Artificial Sequence Synthetic oligonucleotide
auuaaaaacaccuaagugann

SEQ ID NO: 732 - DNA, 21 bp, Artificial Sequence
  Description of Artificial Sequence Synthetic oligonucleotide
ucacuuagguguuuuuaaunn

SEQ ID NO: 733 - DNA, 21 bp, Artificial Sequence
  Description of Artificial Sequence Synthetic oligonucleotide
aaacaccuaagugacuaccnn

SEQ ID NO: 734 - DNA, 21 bp, Artificial Sequence
  Description of Artificial Sequence Synthetic oligonucleotide
gguagucacuuagguguuunn

SEQ ID NO: 735 - DNA, 21 bp, Artificial Sequence
  Description of Artificial Sequence Synthetic oligonucleotide
gacuaccacuuauuucuaann

SEQ ID NO: 736 - DNA, 21 bp, Artificial Sequence
  Description of Artificial Sequence Synthetic oligonucleotide
uuagaaauaagugguagucnn

SEQ ID NO: 737 - DNA, 21 bp, Artificial Sequence
  Description of Artificial Sequence Synthetic oligonucleotide
uuauuucuaaauccucacunn

SEQ ID NO: 738 - DNA, 21 bp, Artificial Sequence
  Description of Artificial Sequence Synthetic oligonucleotide
agugaggauuuagaaauaann

SEQ ID NO: 739 - DNA, 21 bp, Artificial Sequence
  Description of Artificial Sequence Synthetic oligonucleotide
cagaaguuguuagugauuunn

SEQ ID NO: 740 - DNA, 21 bp, Artificial Sequence
  Description of Artificial Sequence Synthetic oligonucleotide
aaaucacuaacaacuucugnn

SEQ ID NO: 741 - DNA, 21 bp, Artificial Sequence
  Description of Artificial Sequence Synthetic oligonucleotide
uuauaagauuuuuaggugunn

SEQ ID NO: 742 - DNA, 21 bp, Artificial Sequence
  Description of Artificial Sequence Synthetic oligonucleotide
acaccuaaaaaucuuauaann

SEQ ID NO: 743 - DNA, 21 bp, Artificial Sequence
  Description of Artificial Sequence Synthetic oligonucleotide
uauaagauuuuuaggugucnn

SEQ ID NO: 744 - DNA, 21 bp, Artificial Sequence
  Description of Artificial Sequence Synthetic oligonucleotide
gacaccuaaaaaucuuauann

SEQ ID NO: 745 - DNA, 21 bp, Artificial Sequence
  Description of Artificial Sequence Synthetic oligonucleotide
uaagaauaaugacguauugnn

SEQ ID NO: 746 - DNA, 21 bp, Artificial Sequence
  Description of Artificial Sequence Synthetic oligonucleotide
caauacgucauuauucuuann

SEQ ID NO: 747 - DNA, 21 bp, Artificial Sequence
  Description of Artificial Sequence Synthetic oligonucleotide
aacuaugcaccuauaaauann

SEQ ID NO: 748 - DNA, 21 bp, Artificial Sequence
  Description of Artificial Sequence Synthetic oligonucleotide
uauuuauaggugcauaguunn

SEQ ID NO: 749 - DNA, 21 bp, Artificial Sequence
  Description of Artificial Sequence Synthetic oligonucleotide
aaauuuuaccauuuugcgann

SEQ ID NO: 750 - DNA, 21 bp, Artificial Sequence
  Description of Artificial Sequence Synthetic oligonucleotide
ucgcaaaaugguaaaauuunn

SEQ ID NO: 751 - DNA, 21 bp, Artificial Sequence
  Description of Artificial Sequence Synthetic oligonucleotide
aauuuuaccauuuugcgaunn

SEQ ID NO: 752 - DNA, 21 bp, Artificial Sequence
  Description of Artificial Sequence Synthetic oligonucleotide
aucgcaaaaugguaaaauunn

SEQ ID NO: 753 - DNA, 21 bp, Artificial Sequence
  Description of Artificial Sequence Synthetic oligonucleotide
uuuuaccauuuugcgaugunn

SEQ ID NO: 754 - DNA, 21 bp, Artificial Sequence
  Description of Artificial Sequence Synthetic oligonucleotide
acaucgcaaaaugguaaaann

SEQ ID NO: 755 - DNA, 21 bp, Artificial Sequence
  Description of Artificial Sequence Synthetic oligonucleotide
uugcgauguguuuuauucann

SEQ ID NO: 756 - DNA, 21 bp, Artificial Sequence
  Description of Artificial Sequence Synthetic oligonucleotide
ugaauaaaacacaucgcaann

SEQ ID NO: 757 - DNA, 21 bp, Artificial Sequence
  Description of Artificial Sequence Synthetic oligonucleotide
ugcgauguguuuuauucacnn

SEQ ID NO: 758 - DNA, 21 bp, Artificial Sequence
  Description of Artificial Sequence Synthetic oligonucleotide
gugaauaaaacacaucgcann

SEQ ID NO: 759 - DNA, 21 bp, Artificial Sequence
  Description of Artificial Sequence Synthetic oligonucleotide
cguuaucucauugcaaaaann

SEQ ID NO: 760 - DNA, 21 bp, Artificial Sequence
  Description of Artificial Sequence Synthetic oligonucleotide
uuuuugcaaugagauaacgnn

SEQ ID NO: 761 - DNA, 21 bp, Artificial Sequence
  Description of Artificial Sequence Synthetic oligonucleotide
uaucccaucucacuuuaaunn

SEQ ID NO: 762 - DNA, 21 bp, Artificial Sequence
  Description of Artificial Sequence Synthetic oligonucleotide
auuaaagugagaugggauann

SEQ ID NO: 763 - DNA, 21 bp, Artificial Sequence
  Description of Artificial Sequence Synthetic oligonucleotide
aucccaucucacuuuaauann

SEQ ID NO: 764 - DNA, 21 bp, Artificial Sequence
  Description of Artificial Sequence Synthetic oligonucleotide
uauuaaagugagaugggaunn

SEQ ID NO: 765 - DNA, 21 bp, Artificial Sequence
  Description of Artificial Sequence Synthetic oligonucleotide
caucucacuuuaauaauaann

SEQ ID NO: 766 - DNA, 21 bp, Artificial Sequence
  Description of Artificial Sequence Synthetic oligonucleotide
uuauuauuaaagugagaugnn

SEQ ID NO: 767 - DNA, 21 bp, Artificial Sequence
  Description of Artificial Sequence Synthetic oligonucleotide
aaaucaugcuuauaagcaann

SEQ ID NO: 768 - DNA, 21 bp, Artificial Sequence
  Description of Artificial Sequence Synthetic oligonucleotide
uugcuuauaagcaugauuunn

SEQ ID NO: 769 - DNA, 21 bp, Artificial Sequence
  Description of Artificial Sequence Synthetic oligonucleotide
augcuuauaagcaacaugann

SEQ ID NO: 770 - DNA, 21 bp, Artificial Sequence
  Description of Artificial Sequence Synthetic oligonucleotide
ucauguugcuuauaagcaunn

SEQ ID NO: 771 - DNA, 21 bp, Artificial Sequence
  Description of Artificial Sequence Synthetic oligonucleotide
agaacugacacaaaggacann

SEQ ID NO: 772 - DNA, 21 bp, Artificial Sequence
  Description of Artificial Sequence Synthetic oligonucleotide
uguccuuugugucaguucunn

SEQ ID NO: 773 - DNA, 21 bp, Artificial Sequence
  Description of Artificial Sequence Synthetic oligonucleotide
auaaaguuauuaauagccann

SEQ ID NO: 774 - DNA, 21 bp, Artificial Sequence
  Description of Artificial Sequence Synthetic oligonucleotide
uggcuauuaauaacuuuaunn

SEQ ID NO: 775 - DNA, 21 bp, Artificial Sequence
  Description of Artificial Sequence Synthetic oligonucleotide
aaaguuauuaauagccauunn

SEQ ID NO: 776 - DNA, 21 bp, Artificial Sequence
  Description of Artificial Sequence Synthetic oligonucleotide
aauggcuauuaauaacuuunn

SEQ ID NO: 777 - DNA, 21 bp, Artificial Sequence
  Description of Artificial Sequence Synthetic oligonucleotide
ggauguauucaugaaaggann

SEQ ID NO: 778 - DNA, 21 bp, Artificial Sequence
  Description of Artificial Sequence Synthetic oligonucleotide
uccuuucaugaauacauccnn

SEQ ID NO: 779 - DNA, 21 bp, Artificial Sequence
  Description of Artificial Sequence Synthetic oligonucleotide
guucucuauguaggcuccann

SEQ ID NO: 780 - DNA, 21 bp, Artificial Sequence
  Description of Artificial Sequence Synthetic oligonucleotide
uggagccuacauagagaacnn

SEQ ID NO: 781 - DNA, 21 bp, Artificial Sequence
  Description of Artificial Sequence Synthetic oligonucleotide
guuccauccuguacaagugnn

SEQ ID NO: 782 - DNA, 21 bp, Artificial Sequence
  Description of Artificial Sequence Synthetic oligonucleotide
cacuuguacaggauggaacnn

SEQ ID NO: 783 - DNA, 21 bp, Artificial Sequence
  Description of Artificial Sequence Synthetic oligonucleotide
cuaaauccucacuauuuuunn

SEQ ID NO: 784 - DNA, 21 bp, Artificial Sequence
  Description of Artificial Sequence Synthetic oligonucleotide
aaaaauagugaggauuuagnn

SEQ ID NO: 785 - DNA, 21 bp, Artificial Sequence
  Description of Artificial Sequence Synthetic oligonucleotide
cuauguaggcuccaaaaccnn

SEQ ID NO: 786 - DNA, 21 bp, Artificial Sequence
  Description of Artificial Sequence Synthetic oligonucleotide
gguuuuggagccuacauagnn

SEQ ID NO: 787 - DNA, 21 bp, Artificial Sequence
  Description of Artificial Sequence Synthetic oligonucleotide
ggugcaugguguggcaacann

SEQ ID NO: 788 - DNA, 21 bp, Artificial Sequence
  Description of Artificial Sequence Synthetic oligonucleotide
uguugccacaccaugcaccnn

SEQ ID NO: 789 - DNA, 21 bp, Artificial Sequence
  Description of Artificial Sequence Synthetic oligonucleotide
ugagaucugcugacagaugnn

SEQ ID NO: 790 - DNA, 21 bp, Artificial Sequence
  Description of Artificial Sequence Synthetic oligonucleotide
caucugucagcagaucucann

SEQ ID NO: 791 - DNA, 21 bp, Artificial Sequence
  Description of Artificial Sequence Synthetic oligonucleotide
cccacucagcauuucggugnn

SEQ ID NO: 792 - DNA, 21 bp, Artificial Sequence
  Description of Artificial Sequence Synthetic oligonucleotide
caccgaaaugcugagugggnn

SEQ ID NO: 793 - DNA, 21 bp, Artificial Sequence
  Description of Artificial Sequence Synthetic oligonucleotide
caugguagcagggucuuugnn

SEQ ID NO: 794 - DNA, 21 bp, Artificial Sequence
  Description of Artificial Sequence Synthetic oligonucleotide
caaagacccugcuaccaugnn

SEQ ID NO: 795 - DNA, 21 bp, Artificial Sequence
  Description of Artificial Sequence Synthetic oligonucleotide
agaauuaaaauaaaacguunn

SEQ ID NO: 796 - DNA, 21 bp, Artificial Sequence
  Description of Artificial Sequence Synthetic oligonucleotide
aacguuuuauuuuaauucunn

SEQ ID NO: 797 - DNA, 21 bp, Artificial Sequence
  Description of Artificial Sequence Synthetic oligonucleotide
aucucgaagucuuccaucann

SEQ ID NO: 798 - DNA, 21 bp, Artificial Sequence
  Description of Artificial Sequence Synthetic oligonucleotide
ugauggaagacuucgagaunn

SEQ ID NO: 799 - DNA, 21 bp, Artificial Sequence
  Description of Artificial Sequence Synthetic oligonucleotide
gugagcaugaaacuaugcann

SEQ ID NO: 800 - DNA, 21 bp, Artificial Sequence
  Description of Artificial Sequence Synthetic oligonucleotide
ugcauaguuucaugcucacnn

SEQ ID NO: 801 - RNA, 21 nt, Artificial Sequence
  Description of Artificial Sequence Synthetic oligonucleotide
gguguggcaacaguggcugag

SEQ ID NO: 802 - RNA, 23 nt, Artificial Sequence
  Description of Artificial Sequence Synthetic oligonucleotide
cucagccacuguugccacaccau

SEQ ID NO: 803 - RNA, 21 nt, Artificial Sequence
  Description of Artificial Sequence Synthetic oligonucleotide
aacaguggcugagaagaccaa

SEQ ID NO: 804 - RNA, 23 nt, Artificial Sequence
  Description of Artificial Sequence Synthetic oligonucleotide
uuggucuucucagccacuguugc

SEQ ID NO: 805 - RNA, 21 nt, Artificial Sequence
  Description of Artificial Sequence Synthetic oligonucleotide
auugcagcagccacuggcuuu

SEQ ID NO: 806 - RNA, 23 nt, Artificial Sequence
  Description of Artificial Sequence Synthetic oligonucleotide
aaagccaguggcugcugcaaugc

SEQ ID NO: 807 - RNA, 21 nt, Artificial Sequence
  Description of Artificial Sequence Synthetic oligonucleotide
aagugacaaauguuggaggag

SEQ ID NO: 808 - RNA, 23 nt, Artificial Sequence
  Description of Artificial Sequence Synthetic oligonucleotide
caccuccaacauuugucacuugc

SEQ ID NO: 809 - RNA, 21 nt, Artificial Sequence
  Description of Artificial Sequence Synthetic oligonucleotide
gaagaaggagccccacaggaa

SEQ ID NO: 810 - RNA, 23 nt, Artificial Sequence
  Description of Artificial Sequence Synthetic oligonucleotide
uuccuguggggcuccuucuucau

SEQ ID NO: 811 - DNA, 21 bp, Artificial Sequence
  Description of Artificial Sequence Synthetic oligonucleotide
cgggugugacagcaguagctt

SEQ ID NO: 812 - DNA, 21 bp, Artificial Sequence
  Description of Artificial Sequence Synthetic oligonucleotide
gcuacugcugucacacccgtt

SEQ ID NO: 813 - DNA, 21 bp, Artificial Sequence
  Description of Artificial Sequence Synthetic oligonucleotide
uccugacaaugaggcuuautt

SEQ ID NO: 814 - DNA, 21 bp, Artificial Sequence
  Description of Artificial Sequence Synthetic oligonucleotide
auaagccucauugucaggatt

SEQ ID NO: 815 - DNA, 21 bp, Artificial Sequence
  Description of Artificial Sequence Synthetic oligonucleotide
uccugacaaugaggcuuautt

SEQ ID NO: 816 - DNA, 21 bp, Artificial Sequence
  Description of Artificial Sequence Synthetic oligonucleotide
auaagccucauugucaggatt

SEQ ID NO: 817 - DNA, 21 bp, Artificial Sequence
  Description of Artificial Sequence Synthetic oligonucleotide
cuacgaaccugaagccuaatt

SEQ ID NO: 818 - DNA, 21 bp, Artificial Sequence
  Description of Artificial Sequence Synthetic oligonucleotide
uuaggcuucagguucguagtt

SEQ ID NO: 819 - DNA, 21 bp, Artificial Sequence
  Description of Artificial Sequence Synthetic oligonucleotide
cuacgaaccugaagccuaatt

SEQ ID NO: 820 - DNA, 21 bp, Artificial Sequence
  Description of Artificial Sequence Synthetic oligonucleotide
uuaggcuucagguucguagtt

SEQ ID NO: 821 - DNA, 21 bp, Artificial Sequence
  Description of Artificial Sequence Synthetic oligonucleotide
cuacgaaccugaagccuaatt

SEQ ID NO: 822 - DNA, 21 bp, Artificial Sequence
  Description of Artificial Sequence Synthetic oligonucleotide
uuaggcuucagguucguagtt

SEQ ID NO: 823 - DNA, 21 bp, Artificial Sequence
  Description of Artificial Sequence Synthetic oligonucleotide
cuauuguagaguggucuautt

SEQ ID NO: 824 - DNA, 21 bp, Artificial Sequence
  Description of Artificial Sequence Synthetic oligonucleotide
auagaccacucuacaauagtt

SEQ ID NO: 825 - RNA, 21 nt, Artificial Sequence
  Description of Artificial Sequence Synthetic oligonucleotide
gaacugugugugagagguccu

SEQ ID NO: 826 - RNA, 23 nt, Artificial Sequence
  Description of Artificial Sequence Synthetic oligonucleotide
aggaccucucacacacaguuccc

SEQ ID NO: 827 - RNA, 21 nt, Artificial Sequence
  Description of Artificial Sequence Synthetic oligonucleotide
gacguaaacggccacaaguuc

SEQ ID NO: 828 - RNA, 21 nt, Artificial Sequence
  Description of Artificial Sequence Synthetic oligonucleotide
acuuguggccguuuacgucgc

SEQ ID NO: 829 - DNA, 21 bp, Artificial Sequence
  Description of Artificial Sequence Synthetic oligonucleotide
cuaugagccugaagccuaatt

SEQ ID NO: 830 - DNA, 21 bp, Artificial Sequence
  Description of Artificial Sequence Synthetic oligonucleotide
uuaggcuucaggcucauagtt

SEQ ID NO: 831 - DNA, 21 bp, Artificial Sequence
  Description of Artificial Sequence Synthetic oligonucleotide
aacagtggctgagaagaccaa

SEQ ID NO: 832 - DNA, 21 bp, Artificial Sequence
  Description of Artificial Sequence Synthetic oligonucleotide
ttcagtggctgaaaaaaccaa

SEQ ID NO: 833 - DNA, 19 bp, Artificial Sequence
  Description of Artificial Sequence Synthetic oligonucleotide
tcctgacaatgaggcttat

SEQ ID NO: 834 - DNA, 19 bp, Artificial Sequence
  Description of Artificial Sequence Synthetic oligonucleotide
tgaggacccaccccaggag

SEQ ID NO: 835 - DNA, 19 bp, Artificial Sequence
  Description of Artificial Sequence Synthetic oligonucleotide
ctacgaacctgaagcctaa

SEQ ID NO: 836 - DNA, 21 bp, Artificial Sequence
  Description of Artificial Sequence Synthetic oligonucleotide
aacagtggctgagaagaccaa

SEQ ID NO: 837 - DNA, 21 bp, Artificial Sequence
  Description of Artificial Sequence Synthetic oligonucleotide
aacagtggctgagaagaccaa

SEQ ID NO: 838 - DNA, 19 bp, Artificial Sequence
  Description of Artificial Sequence Synthetic oligonucleotide
tcctgacaatgaggcttat

SEQ ID NO: 839 - DNA, 19 bp, Artificial Sequence
  Description of Artificial Sequence Synthetic oligonucleotide
tcctggcagtgaggcttat

SEQ ID NO: 840 - DNA, 19 bp, Artificial Sequence
  Description of Artificial Sequence Synthetic oligonucleotide
ctacgaacctgaagcctaa

SEQ ID NO: 841 - DNA, 19 bp, Artificial Sequence
  Description of Artificial Sequence Synthetic oligonucleotide
ctatgagcctgaagcctaa

SEQ ID NO: 842 - DNA, 21 bp, Artificial Sequence
  Description of Artificial Sequence Synthetic oligonucleotide
cuuacgcugaguacuucgatt

SEQ ID NO: 843 - DNA, 21 bp, Artificial Sequence
  Description of Artificial Sequence Synthetic oligonucleotide
ucgaaguacucagcguaagtt

SEQ ID NO: 844 - DNA, 21 bp, Artificial Sequence
  Description of Artificial Sequence Synthetic oligonucleotide
cuuacgcugaguacuucgatt

SEQ ID NO: 845 - DNA, 21 bp, Artificial Sequence
  Description of Artificial Sequence Synthetic oligonucleotide
ucgaaguacucagcguaagtt

SEQ ID NO: 846 - DNA, 21 bp, Artificial Sequence
  Description of Artificial Sequence Synthetic oligonucleotide
cuaugagccugaagccuaatt

SEQ ID NO: 847 - DNA, 21 bp, Artificial Sequence
  Description of Artificial Sequence Synthetic oligonucleotide
uuaggcuucaggcucauagtt

SEQ ID NO: 848 - DNA, 21 bp, Artificial Sequence
  Description of Artificial Sequence Synthetic oligonucleotide
cuaugagccugaagccuaatt

SEQ ID NO: 849 - DNA, 21 bp, Artificial Sequence
  Description of Artificial Sequence Synthetic oligonucleotide
uuaggcuucaggcucauagtt

SEQ ID NO: 850 - DNA, 21 bp, Artificial Sequence
  Description of Artificial Sequence Synthetic oligonucleotide
cuuacgcugaguacuucgatt

SEQ ID NO: 851 - DNA, 21 bp, Artificial Sequence
  Description of Artificial Sequence Synthetic oligonucleotide
ucgaaguacucagcguaagtt

SEQ ID NO: 852 - DNA, 21 bp, Artificial Sequence
  Description of Artificial Sequence Synthetic oligonucleotide
cuaugagccugaagccuaatt

SEQ ID NO: 853 - DNA, 21 bp, Artificial Sequence
  Description of Artificial Sequence Synthetic oligonucleotide
uuaggcuucaggcucauagtt

SEQ ID NO: 854 - DNA, 36 bp, Artificial Sequence
  Description of Artificial Sequence Synthetic oligonucleotide
ggtcttctcagccactgttgtcactccatgaaccac

(19) United States
(12) Patent Application Publication
(10) Pub. No.: US 2009/0176729 A1
(43) Pub. Date: Jul. 9, 2009

(54) METHOD OF TREATING NEURODEGENERATIVE DISEASE

(75) Inventor: Pamela Tan, Kulmbach (DE)

Correspondence Address: LOWRIE, LANDO & ANASTASI, LLP, ONE MAIN STREET, SUITE 1100, CAMBRIDGE, MA, 02142, US

(73) Assignee: ALNYLAM PHARMACEUTICALS, INC., Cambridge, MA (US)

(21) Appl. No.: 12/334,080

(22) Filed: Dec. 12, 2008

Related U.S. Application Data
(60) Provisional application No. 61/013,759, filed on Dec. 14, 2007.
(60) Provisional application No. 61/058,468, filed on Jun. 3, 2008.

(51) Int. Cl. A61K 31/713; C07H 21/02; C12N 5/10; C12N 15/87; C12N 15/63

(52) U.S. Cl. 514/44; 536/23.1; 435/325; 435/463; 435/320.1

(57) ABSTRACT

Aspects featured in the invention relate to compositions and methods for inhibiting alpha-synuclein (SNCA) gene expression, such as for the treatment of neurodegenerative disorders. An anti-SNCA agent featured herein that targets the SNCA gene can have been modified to alter distribution in favor of neural cells.

### RELATED APPLICATIONS

This application claims the benefit of U.S. Provisional Application No. 61/013,759, filed Dec. 14, 2007, and U.S. Provisional Application No. 61/058,468, filed Jun. 3, 2008. Both prior applications are incorporated herein by reference in their entirety.

### TECHNICAL FIELD

This invention relates to methods and compositions for treating neurodegenerative disease, and more particularly to the downregulation of the alpha-synuclein gene for the treatment of synucleinopathies.

### BACKGROUND

RNA interference or “RNAi” is a term initially coined by Fire and co-workers to describe the observation that double-stranded RNA (dsRNA) can block gene expression when it is introduced into worms (Fire et al., Nature 391:806-811, 1998). Short dsRNA directs gene-specific, post-transcriptional silencing in many organisms, including vertebrates, and has provided a new tool for studying gene function.

Expression of the SNCA gene produces the protein alpha-synuclein. Mutations in the SNCA gene and SNCA gene multiplications have been linked to familial Parkinson's disease (PD). PD patients demonstrate alpha-synuclein protein aggregates in the brain. Similar aggregates are observed in patients diagnosed with sporadic PD, Alzheimer's Disease, multiple system atrophy, and Lewy body dementia.

### SUMMARY

Aspects of the invention relate to compositions for inhibiting alpha-synuclein (SNCA) expression, and methods of using those compositions.

In one aspect, the invention features a method of treating a subject by administering a dsRNA that inhibits expression of SNCA. In one embodiment, the subject is a mammal, such as a human, e.g., a subject diagnosed as having, or at risk for developing, a neurodegenerative disorder. The inhibition can be effected at any level, e.g., at the level of transcription, the level of translation, or post-translationally. Tables 2, 3 and 4 describe dsRNA that can be used to inhibit SNCA expression.

In one embodiment the inhibitory agent is a dsRNA that targets an SNCA nucleic acid, e.g., an SNCA RNA. The dsRNA has an antisense strand complementary to a nucleotide sequence of an SNCA RNA, and a sense strand sufficiently complementary to hybridize to the antisense strand. In one embodiment, the dsRNA includes a modification that stabilizes the dsRNA in a biological sample. For example, the modified dsRNA is less susceptible to degradation, e.g., less susceptible to cleavage by an exo- or endonuclease. The dsRNA can include, for example, at least one 5′-uridine-adenine-3′ (5′-UA-3′) dinucleotide wherein the uridine is a 2′-modified nucleotide, or at least one 5′-uridine-guanine-3′ (5′-UG-3′) dinucleotide, wherein the 5′-uridine is a 2′-modified nucleotide, or at least one 5′-cytidine-adenine-3′ (5′-CA-3′) dinucleotide, wherein the 5′-cytidine is a 2′-modified nucleotide, or at least one 5′-uridine-uridine-3′ (5′-UU-3′) dinucleotide, wherein the 5′-uridine is a 2′-modified nucleotide, or at least one 5′-cytidine-cytidine-3′ (5′-CC-3′) dinucleotide, wherein the 5′-cytidine is a 2′-modified nucleotide. The dsRNA can include at least 2, at least 3, at least 4 or at least 5 of the dinucleotides. In one embodiment, the 2′-modified nucleotide is a 2′-O-methylated nucleotide. In another embodiment the dsRNA includes a phosphorothioate.

In another embodiment, the dsRNA is at least 21 nucleotides long and includes a sense RNA strand and an antisense RNA strand, wherein the antisense RNA strand is 25 or fewer nucleotides in length, and the duplex region of the dsRNA is 18-25 nucleotides in length, e.g., 19-24 nucleotides in length. In some embodiments, the dsRNA is from about 10 to about 15 nucleotides, and in other embodiments the dsRNA is from about 25 to about 30 nucleotides in length, The dsRNA may further include a nucleotide overhang having 1 to 4 unpaired nucleotides, and the unpaired nucleotides may have at least one phosphorothioate dinucleotide linkage. The nucleotide overhang can be, e.g., at the 3′ end of the antisense strand of the dsRNA. In another embodiment, the antisense strand of the dsRNA includes or consists of the nucleotide sequence of an antisense strand shown in Tables 2, 3, or 4. In another embodiment, the sense strand of the dsRNA includes or consists of the nucleotide sequence of a sense strand shown in Tables 2, 3, or 4. In yet another embodiment, the antisense strand of the dsRNA overlaps the nucleotide sequence of an antisense strand shown in Tables 2, 3, or 4, e.g., by at least 1, 5, 10, 11, 12, 13, 14, 15, 16, 17, 18, 19, 20, 21, 22, 23, or 24 nucleotides. Likewise, the sense strand of the dsRNA can overlap the nucleotide sequence of a sense strand shown in Tables 2, 3, or 4, e.g., by at least 1, 5, 10, 11, 12, 13, 14, 15, 16, 17, 18, 19, 20, 21, 22, 23, or 24 nucleotides. In one embodiment, the SNCA dsRNA is formulated in a stable nucleic acid particle (SNALP).

In another embodiment, the dsRNA includes at least two sequences that are substantially complementary to each other. A sense strand of the dsRNA includes a first sequence, and an antisense strand of the dsRNA includes a second sequence. The second sequence has a region that is substantially complementary to the corresponding region of an mRNA encoding SNCA, and this corresponding region is less than 30 nucleotides in length. In one embodiment, the first sequence of the dsRNA is one of the sense strand sequences listed in Tables 2, 3, and 4, and the second sequence is one of the antisense strand sequences listed in Tables 2, 3, and 4.

In another embodiment, the dsRNA targets a wildtype SNCA nucleic acid, and in yet another embodiment, the dsRNA targets a polymorphism or mutation of SNCA. For example, the dsRNA can target a mutation in a codon of the SNCA open reading frame that corresponds to an A53T, A30P, or E46K mutation. In some embodiments, the dsRNA targets the 3′UTR or the 5′UTR of SNCA. In some embodiments, the dsRNA targets a spliced isoform of SNCA.

In one embodiment, the dsRNA can reduce mRNA levels by at least 40%, 60%, 80%, or 90%, e.g., as measured by an assay. Assays to measure SNCA mRNA and protein levels can be performed by standard methods known in the art. For example, SNCA mRNA can be measured by RT-PCR or Northern blot analysis. SNCA protein levels can be measured by enzymatic assay, or by antibody-based methods, e.g., Western blot, ELISA, or immunohistochemistry.

The SNCA gene can be a target for treatment methods of neurodegenerative disease. In one embodiment, a dsRNA described in Tables 2, 3, or 4 can be used to target an SNCA nucleic acid. A combination of therapies to downregulate SNCA expression and activity can also be used.

In some embodiments, the subject (e.g., the human) carries a multiplication (e.g., a duplication or triplication) of the SNCA gene, or a genetic variation in the Parkin or ubiquitin carboxy-terminal hydrolase L1 (UCHL1) gene. In another embodiment, the subject is diagnosed with a synucleinopathy. The synucleinopathy is characterized by the aggregation of alpha-synuclein monomers. A dsRNA can be administered to a human diagnosed as having, e.g., Parkinson's disease (PD), Alzheimer's disease, multiple system atrophy, Lewy body dementia, or a retinal disorder, e.g., a retinopathy.

In another embodiment, the dsRNA is at least 21 nucleotides long and includes a sense RNA strand and an antisense RNA strand, wherein the antisense RNA strand is 25 or fewer nucleotides in length, and the duplex region of the dsRNA is 18-25 nucleotides in length, e.g., 19-24 nucleotides in length. In some embodiments, the dsRNA is from about 10 to about 15 nucleotides, and in other embodiments the dsRNA is from about 25 to about 30 nucleotides. The dsRNA may further include a nucleotide overhang having 1 to 4 unpaired nucleotides, and the unpaired nucleotides may have at least one phosphorothioate dinucleotide linkage. The nucleotide overhang can be, e.g., at the 3′ end of the antisense strand of the dsRNA. In one embodiment, the SNCA dsRNA is formulated in a stable nucleic acid particle (SNALP).

In another aspect, the invention features a dsRNA, e.g., a dsRNA described herein, e.g., in Tables 2, 3, or 4, that targets an SNCA nucleic acid, e.g., an SNCA RNA.

In another aspect, the invention features a pharmaceutical composition of a dsRNA, e.g., a dsRNA described herein, e.g., in Table 2, 3, or 4, and a pharmaceutically acceptable carrier. In a one embodiment the pharmaceutical composition does not include another agent which silences gene expression. In another embodiment the pharmaceutical composition does not include another dsRNA, e.g., a dsRNA of a length or overhang structure described herein. In yet another embodiment the pharmaceutical composition consists of or consists essentially of the subject dsRNA. In another embodiment the pharmaceutical composition includes more than one but not more than 2, 3 or 4 dsRNAs.

In one embodiment, the pharmaceutical composition is disposed in a device configured to provide localized delivery to the brain, such as into the substantia nigra, hippocampus or cortex of the brain. Delivery can be, for example, by infusion, e.g., by intraparenchymal infusion.

In another embodiment, a compositions containing a dsRNA targeting SNCA is administered to a patient, and after 1, 2, 3, or 4 weeks, the patient is tested to determine SNCA mRNA levels, e.g., in the blood or urine, or in a particular tissue. If the level of SNCA mRNA is determined to be above a pre-set level, the patient will be administered another dose of SNCA dsRNA. If the level of SNCA mRNA is determined to be below the pre-set level, the patient is not administered another dose of the SNCA dsRNA.

It has been discovered that a single administration can provide prolonged silencing. Thus, in another embodiment, a dose of SNCA dsRNA is administered to a patient and the dose is sufficient to downregulate SNCA mRNA or protein levels to a state that is less than or equal to 20% of pretreatment levels (or levels that would be seen in the absence of treatment) for at least 5, 10, or 15 days post-treatment; less than or equal to 40% of pretreatment levels (or levels that would be seen in the absence of treatment) for at least 5, 10, or 15 days post-treatment; less than or equal to 60% of pretreatment levels (or levels that would be seen in the absence of treatment) for at least 5, 10, 15, or 20 days post-treatment; or less than or equal to 80% of pretreatment levels (or levels that would be seen in the absence of treatment) for at least 5, 10, 15, 20, or 25 days post-treatment.

In one embodiment, a first dose of SNCA dsRNA is administered, and no subsequent dose of SNCA dsRNA is administered for at least 5, 10, 15, 20 or 30 days after the first dose. In another embodiment, a subsequent dose is administered but not until at least 5, 10, 15, 20, or 30 days have elapsed since the first dose.

In another embodiment, a patient continues to receive at least one other therapeutic treatment for the synucleinopathy while receiving treatment with SNCA dsRNA. For example, a patient with Parkinson's Disease can continue to receive administration of agent for alleviating symptoms, a neuroprotective agent (e.g., for slowing or halting disease progression), or a restorative agent (e.g., for reversing the disease process). Symptomatic therapies include the drugs carbidopa/levodopa, entacapone, tolcapone, pramipexole, ropinerole, pergolide, bromocriptine, selegeline, amantadine, and several anticholingergic agents. Deep brain stimulation surgery as well as stereotactic brain lesioning may also provide symptomatic relief. Neuroprotective therapies include, for example, carbidopa/levodopa, selegeline, vitamin E, amantadine, pramipexole, ropinerole, coenzyme Q10, and GDNF. Restorative therapies can include, for example, surgical transplantation of stem cells.

In another aspect, the invention features a method of providing instructions, e.g., to a healthcare provider or a patient on the administration of SNCA dsRNA. The method includes: providing instructions to administer to the patient a dose of SNCA dsRNA in a treatment regimen described herein, e.g., a dose followed by at least 21 days within a subsequent dose of SNCA dsRNA.

In another aspect, the invention features a method of selecting or treating a patient in need of SNCA dsRNA to treat a disorder described herein. The method includes selecting a patient on the basis of the patient being in need of decreased SNCA RNA for at least 5, 10, 15, 20 or 30 days, and optionally administering the drug to the patient.

In another aspect, the invention features a method of reducing the amount of SNCA or SNCA RNA in a cell of a subject (e.g., a mammalian subject, such as a human). The method includes contacting the cell with an agent that inhibits the expression of SNCA, e.g., a dsRNA described herein, e.g., in Tables 2, 3, or 4. The inhibition can be effected at any level, e.g., at the level of transcription, the level of translation, or post-translationally.

In another aspect, the invention features a method of making a dsRNA described herein, e.g., in Tables 2, 3, or 4. The method includes selecting a nucleotide sequence of between 18 and 25 nucleotides long from the nucleotide sequence of an SNCA mRNA, and synthesizing the dsRNA. The sense strand of the dsRNA includes the nucleotide sequence selected from SNCA RNA, and the antisense strand is sufficiently complementary to hybridize to the sense strand. In one embodiment, the method further includes administering the dsRNA to a subject (e.g., a mammalian subject, such as a human subject) as described herein.

In another aspect, the invention features a method of evaluating an agent, e.g., an agent of a type described herein, such as a dsRNA agent having an antisense strand shown in Tables 2, 3, or 4, and a sense strand shown in Tables 2, 3, or 4, dsRNA for the ability to inhibit SNCA expression, e.g., an agent that targets an SNCA or SNCA nucleic acid. The method includes: providing a candidate agent and determining, e.g., by the use of one or more of the test systems described herein, if said candidate agent modulates, e.g., inhibits, SNCA expression.

In one embodiment the method includes evaluating the agent in a first test system; and, if a predetermined level of modulation is seen, evaluating the candidate in a second, e.g., a different, test system. In one embodiment the second test system includes administering the candidate agent to an animal and evaluating the effect of the candidate agent on SNCA expression in the animal. In certain embodiments, two test systems are used and the first is a high-throughput system. For example, in such embodiments the first or initial test is used to screen at least 100, 1,000, or 10,000 times more agents than is the second test, e.g., an animal system.

A test system can include: contacting the candidate agent with a target molecule, e.g., an SNCA nucleic acid, e.g., an RNA, such as in vitro, and determining if there is an interaction, e.g., binding of the candidate agent to the target, or modifying the target, e.g., by making or breaking a covalent bond in the target. Modification is correlated with the ability to modulate SNCA expression. The test system can include contacting the candidate agent with a cell and evaluating modulation of SNCA expression. For example, this can include contacting the candidate agent with a cell capable of expressing SNCA or SNCA RNA (from an endogenous gene or from an exogenous construct) and evaluating the level of SNCA or SNCA RNA. In another embodiment, the test system can include contacting the candidate agent with a cell that expresses an RNA or protein from an SNCA control region (e.g., an SNCA control region) linked to a heterologous sequence, e.g., a marker protein, e.g., a fluorescent protein such as GFP, which construct can be either chromosomal or episomal, and determining the effect on RNA or protein levels. The test system can also include contacting the candidate agent, in vitro, with a tissue sample, e.g., a brain tissue sample, e.g., a slice or section, an optical tissue sample, or other sample which includes neural tissue, and evaluating the level of SNCA or SNCA RNA. The test system can include administering the candidate agent, in vivo, to an animal, and evaluating the level of SNCA or SNCA RNA. In any of these the effect of the candidate agent on SNCA expression can include comparing SNCA gene expression with a predetermined standard, such as a control, e.g., an untreated cell, tissue or animal. SNCA gene expression can be compared, e.g., before and after contacting with the candidate agent. The method allows determining whether the dsRNA is useful for inhibiting SNCA gene expression.

In one embodiment, SNCA gene expression can be evaluated by a method to examine SNCA RNA levels (e.g., Northern blot analysis, RT-PCR, or RNAse protection assay) or SNCA protein levels (e.g., Western blot).

In one embodiment, a second test is performed by administering the agent to an animal, e.g., a mammal, such as a mouse, rat, rabbit, human, or non-human primate, and the animal is monitored for an effect of the agent. For example, a tissue of the animal, such as, a brain tissue or ocular tissue, is examined for an effect of the agent on SNCA expression. The tissue can be examined for the presence of SNCA RNA and/or protein, for example. In one embodiment, the animal is observed to monitor an improvement or stabilization of a cognitive symptom. The agent can be administered to the animal by any method, e.g., orally, or by intrathecal or parenchymal injection, such as by stereoscopic injection into the brain. In some embodiments, the agent is administered to the substantia nigra, hippocampus or cortex of the brain.

In one embodiment, the invention features a method of evaluating a dsRNA, e.g., a dsRNA described herein, that targets an SNCA nucleic acid. The method includes providing a dsRNA that targets an SNCA nucleic acid (e.g., an SNCA RNA); contacting the dsRNA with a cell containing, and capable of expressing, an SNCA gene; and evaluating the effect of the dsRNA on SNCA expression, e.g., by comparing SNCA gene expression with a control, e.g., in the cell. SNCA gene expression can be compared, e.g., before and after contacting the dsRNA with the cell. The method allows determining whether the dsRNA is useful for inhibiting SNCA gene expression. For example, the dsRNA can be determined to be useful for inhibiting SNCA gene expression if the dsRNA reduces expression by a predetermined amount, e.g., by 10, 25, 50, 75, or 90%, e.g., as compared with a predetermined reference value, e.g., as compared with the amount of SNCA RNA or protein prior to contacting the dsRNA with the cell. The SNCA gene can be endogenously or exogenously expressed.

The methods and compositions featured in the invention, e.g., the methods and iRNA compositions to treat the neurodegenerative disorders described herein, can be used with any dosage and/or formulation described herein, as well as with any route of administration described herein.

In addition to their presence in the brain, alpha-synuclein polypeptides have been found in ocular tissues, including the retina and optic nerve. Accordingly, the compositions and methods described herein are suitable for treating synucleinopathies of the eye or ocular tissues, including but not limited to retinopathies.

In another aspect, the invention features a container or delivery device that contains a dose of SNCA dsRNA sufficient to decrease SNCA RNA for at least 5, 10, 15, 20 or 30 days.

In yet another aspect, the invention features a single dose, which is sufficient to decrease SNCA RNA for at least 5, 10, 15, 20 or 30 days. In one embodiment, the kit includes a delivery device, such as a delivery device described herein.

Thus, in another aspect, the invention features a method of treating a subject by administering an agent which inhibits the expression of SNCA in the eye or in ocular tissue, e.g., a dsRNA described herein, e.g., in Tables 2, 3, or 4. In one embodiment, the subject is a mammal, such as a human, e.g., a subject diagnosed as having, or at risk for developing a synucleinopathy of the eye, e.g., a retinopathy. The inhibition can be effected at any level, e.g., at the level of transcription, the level of translation, or post-translationally.

A dsRNA that targets an SNCA nucleic acid can be referred to as an anti-SNCA dsRNA.

TABLE 1
siRNAs targeting alpha-synuclein. Sequences
are provided below in Table 2.

| 23mer pos. in | Sense | Antisense | duplex |
|---|---|---|---|
| NM_000345 | strand Name | strand name | name |
| 703-721 | A30326 | A30327 | AD-3357 |
| 569-587 | A30328 | A30329 | AD-3358 |
| 442-460 | A30330 | A30331 | AD-3359 |
| 570-588 | A30332 | A30333 | AD-3360 |
| 1190-1208 | A30334 | A30335 | AD-3361 |
| 702-720 | A30336 | A30337 | AD-3362 |
| 631-649 | A30338 | A30339 | AD-3363 |
| 1193-1211 | A30340 | A30341 | AD-3364 |
| 572-590 | A30342 | A30343 | AD-3365 |
| 574-592 | A30344 | A30345 | AD-3366 |
| 706-724 | A30346 | A30347 | AD-3367 |
| 1192-1210 | A30348 | A30349 | AD-3368 |
| 848-866 | A30350 | A30351 | AD-3369 |
| 444-462 | A30352 | A30353 | AD-3370 |
| 661-679 | A30354 | A30355 | AD-3371 |
| 665-683 | A30356 | A30357 | AD-3372 |
| 742-760 | A30358 | A30359 | AD-3373 |
| 919-937 | A30360 | A30361 | AD-3374 |
| 701-719 | A30362 | A30363 | AD-3375 |
| 705-723 | A30364 | A30365 | AD-3376 |
| 867-885 | A30366 | A30367 | AD-3377 |
| 807-825 | A30368 | A30369 | AD-3378 |
| 710-728 | A30370 | A30371 | AD-3379 |
| 873-891 | A30372 | A30373 | AD-3380 |
| 872-890 | A30374 | A30375 | AD-3381 |
| 260-278 | A30376 | A30377 | AD-3382 |
| 441-459 | A30378 | A30379 | AD-3383 |
| 858-876 | A30380 | A30381 | AD-3384 |
| 1195-1213 | A30382 | A30383 | AD-3385 |
| 145-163 | A30384 | A30385 | AD-3386 |
| 151-169 | A30386 | A30387 | AD-3387 |
| 157-175 | A30388 | A30389 | AD-3388 |
| 437-455 | A30390 | A30391 | AD-3389 |
| 438-456 | A30392 | A30393 | AD-3390 |
| 440-458 | A30394 | A30395 | AD-3391 |
| 461-479 | A30396 | A30397 | AD-3392 |
| 571-589 | A30398 | A30399 | AD-3393 |
| 573-591 | A30400 | A30401 | AD-3394 |
| 629-647 | A30402 | A30403 | AD-3395 |
| 704-722 | A30404 | A30405 | AD-3396 |
| 707-725 | A30406 | A30407 | AD-3397 |
| 711-729 | A30408 | A30409 | AD-3398 |
| 737-755 | A30410 | A30411 | AD-3399 |
| 743-761 | A30412 | A30413 | AD-3400 |
| 792-810 | A30414 | A30415 | AD-3401 |
| 799-817 | A30416 | A30417 | AD-3402 |
| 828-846 | A30418 | A30419 | AD-3403 |
| 869-887 | A30420 | A30421 | AD-3404 |
| 920-938 | A30422 | A30423 | AD-3405 |
| 929-947 | A30424 | A30425 | AD-3406 |
| 960-978 | A30426 | A30427 | AD-3407 |
| 1049-1067 | A30428 | A30429 | AD-3408 |
| 1053-1071 | A30430 | A30431 | AD-3409 |
| 1194-1212 | A30432 | A30433 | AD-3410 |
| 150-168 | A30434 | A30435 | AD-3411 |
| 158-176 | A30436 | A30437 | AD-3412 |
| 109-127 | A30438 | A30439 | AD-3413 |
| 149-167 | A30440 | A30441 | AD-3414 |
| 156-174 | A30442 | A30443 | AD-3415 |
| 233-251 | A30444 | A30445 | AD-3416 |
| 329-347 | A30446 | A30447 | AD-3417 |
| 445-463 | A30448 | A30449 | AD-3418 |
| 633-651 | A30450 | A30451 | AD-3419 |
| 790-808 | A30452 | A30453 | AD-3420 |
| 798-816 | A30454 | A30455 | AD-3421 |
| 800-818 | A30456 | A30457 | AD-3422 |
| 805-823 | A30458 | A30459 | AD-3423 |
| 808-826 | A30460 | A30461 | AD-3424 |
| 868-886 | A30462 | A30463 | AD-3425 |
| 928-946 | A30464 | A30465 | AD-3426 |
| 40-58 | A30466 | A30467 | AD-3427 |
| 41-59 | A30468 | A30469 | AD-3428 |
| 61-79 | A30470 | A30471 | AD-3429 |
| 144-162 | A30472 | A30473 | AD-3430 |
| 148-166 | A30474 | A30475 | AD-3431 |
| 152-170 | A30476 | A30477 | AD-3432 |
| 153-171 | A30478 | A30479 | AD-3433 |
| 154-172 | A30480 | A30481 | AD-3434 |
| 248-266 | A30482 | A30483 | AD-3435 |
| 252-270 | A30484 | A30485 | AD-3436 |
| 443-461 | A30486 | A30487 | AD-3437 |
| 482-500 | A30488 | A30489 | AD-3438 |
| 505-523 | A30490 | A30491 | AD-3439 |
| 540-558 | A30492 | A30493 | AD-3440 |
| 544-562 | A30494 | A30495 | AD-3441 |
| 548-566 | A30496 | A30497 | AD-3442 |
| 566-584 | A30498 | A30499 | AD-3443 |
| 575-593 | A30500 | A30501 | AD-3444 |
| 579-597 | A30502 | A30503 | AD-3445 |
| 598-616 | A30504 | A30505 | AD-3446 |
| 628-646 | A30506 | A30507 | AD-3447 |
| 632-650 | A30508 | A30509 | AD-3448 |
| 660-678 | A30510 | A30511 | AD-3449 |
| 664-682 | A30512 | A30513 | AD-3450 |
| 671-689 | A30514 | A30515 | AD-3451 |
| 699-717 | A30516 | A30517 | AD-3452 |
| 700-718 | A30518 | A30519 | AD-3453 |
| 730-748 | A30520 | A30521 | AD-3454 |
| 731-749 | A30522 | A30523 | AD-3455 |
| 736-754 | A30524 | A30525 | AD-3456 |
| 748-766 | A30526 | A30527 | AD-3457 |
| 757-775 | A30528 | A30529 | AD-3458 |
| 796-814 | A30530 | A30531 | AD-3459 |
| 826-844 | A30532 | A30533 | AD-3460 |
| 827-845 | A30534 | A30535 | AD-3461 |
| 862-880 | A30536 | A30537 | AD-3462 |
| 926-944 | A30538 | A30539 | AD-3463 |
| 954-972 | A30540 | A30541 | AD-3464 |
| 955-973 | A30542 | A30543 | AD-3465 |
| 957-975 | A30544 | A30545 | AD-3466 |
| 967-985 | A30546 | A30547 | AD-3467 |
| 968-986 | A30548 | A30549 | AD-3468 |
| 1024-1042 | A30550 | A30551 | AD-3469 |
| 1054-1072 | A30552 | A30553 | AD-3470 |
| 1055-1073 | A30554 | A30555 | AD-3471 |
| 1059-1077 | A30556 | A30557 | AD-3472 |
| 1078-1096 | A30558 | A30559 | AD-3473 |
| 1083-1101 | A30560 | A30561 | AD-3474 |
| 1106-1124 | A30562 | A30563 | AD-3475 |
| 1130-1148 | A30564 | A30565 | AD-3476 |
| 1132-1150 | A30566 | A30567 | AD-3477 |
| 49-67 | A30568 | A30569 | AD-3478 |
| 155-173 | A30570 | A30571 | AD-3479 |
| 512-530 | A30572 | A30573 | AD-3480 |
| 763-781 | A30574 | A30575 | AD-3481 |
| 160-178 | A30576 | A30577 | AD-3482 |
| 190-208 | A30578 | A30579 | AD-3483 |
| 494-512 | A30580 | A30581 | AD-3484 |
| 627-645 | A30582 | A30583 | AD-3485 |
| 669-687 | A30584 | A30585 | AD-3486 |
| 1009-1027 | A30586 | A30587 | AD-3487 |
| 581-599 | A30588 | A30589 | AD-3488 |
| 916-934 | A30590 | A30591 | AD-3489 |

TABLE 2

siRNAs targeting alpha-synuclein.
When the sense and antisense strands
are annealed, each of the resulting
siRNAs has a TT overhang at each end.

| | | | | | |
|---|---|---|---|---|---|
| Sense | SEQ | | Antisense | SEQ | |
| strand | ID | Sense strand | strand | ID | Antisense strand |
| name | NO: | sequence (5′-3′) | name | NO: | sequence (5′-3′) |

| | | | | | |
|---|---|---|---|---|---|
| A30326 | 3 | GUGGCUUCAAUCUACGAUGTT | A30327 | 4 | CAUCGUAGAUUGAAGCCACTT |
| A30328 | 5 | UUUUUACAGUGUAUCUCGATT | A30329 | 6 | UCGAGAUACACUGUAAAAATT |
| A30330 | 7 | GUAUCAAGACUACGAACCUTT | A30331 | 8 | AGGUUCGUAGUCUUGAUACTT |
| A30332 | 9 | UUUUACAGUGUAUCUCGAATT | A30333 | 10 | UUCGAGAUACACUGUAAAATT |
| A30334 | 11 | ACAUUAACCCUACACUCGGTT | A30335 | 12 | CCGAGUGUAGGGUUAAUGUTT |
| A30336 | 13 | UGUGGCUUCAAUCUACGAUTT | A30337 | 14 | AUCGUAGAUUGAAGCCACATT |
| A30338 | 15 | CUCAGCAUUUCGGUGCUUCTT | A30339 | 16 | GAAGCACCGAAAUGCUGAGTT |
| A30340 | 17 | UUAACCCUACACUCGGAAUTT | A30341 | 18 | AUUCCGAGUGUAGGGUUAATT |
| A30342 | 19 | UUACAGUGUAUCUCGAAGUTT | A30343 | 20 | ACUUCGAGAUACACUGUAATT |
| A30344 | 21 | ACAGUGUAUCUCGAAGUCUTT | A30345 | 22 | AGACUUCGAGAUACACUGUTT |
| A30346 | 23 | GCUUCAAUCUACGAUGUUATT | A30347 | 24 | UAACAUCGUAGAUUGAAGCTT |
| A30348 | 25 | AUUAACCCUACACUCGGAATT | A30349 | 26 | UUCCGAGUGUAGGGUUAAUTT |
| A30350 | 27 | UUAAUGAUACUGUCUAAGATT | A30351 | 28 | UCUUAGACAGUAUCAUUAATT |
| A30352 | 29 | AUCAAGACUACGAACCUGATT | A30353 | 30 | UCAGGUUCGUAGUCUUGAUTT |
| A30354 | 31 | AGUGAAUACAUGGUAGCAGTT | A30355 | 32 | CUGCUACCAUGUAUUCACUTT |
| A30356 | 33 | AAUACAUGGUAGCAGGGUCTT | A30357 | 34 | GACCCUGCUACCAUGUAUUTT |
| A30358 | 35 | CUAAGUGACUACCACUUAUTT | A30359 | 36 | AUAAGUGGUAGUCACUUAGTT |
| A30360 | 37 | AGCAUGAAACUAUGCACCUTT | A30361 | 38 | AGGUGCAUAGUUUCAUGCUTT |
| A30362 | 39 | UUGUGGCUUCAAUCUACGATT | A30363 | 40 | UCGUAGAUUGAAGCCACAATT |
| A30364 | 41 | GGCUUCAAUCUACGAUGUUTT | A30365 | 42 | AACAUCGUAGAUUGAAGCCTT |
| A30366 | 43 | AUAAUGACGUAUUGUGAAATT | A30367 | 44 | UUUCACAAUACGUCAUUAUTT |
| A30368 | 45 | AGUGAUUUGCUAUCAUAUATT | A30369 | 46 | UAUAUGAUAGCAAAUCACUTT |
| A30370 | 47 | CAAUCUACGAUGUUAAAACTT | A30371 | 48 | GUUUUAACAUCGUAGAUUGTT |
| A30372 | 49 | ACGUAUUGUGAAAUUUGUUTT | A30373 | 50 | AACAAAUUUCACAAUACGUTT |
| A30374 | 51 | GACGUAUUGUGAAAUUUGUTT | A30375 | 52 | ACAAAUUUCACAAUACGUCTT |
| A30376 | 53 | ACGGGUGUGACAGCAGUAGTT | A30377 | 54 | CUACUGCUGUCACACCOGUTT |
| A30378 | 55 | GGUAUCAAGACUACGAACCTT | A30379 | 56 | GGUUCGUAGUCUUGAUACCTT |
| A30380 | 57 | UGUCUAAGAAUAAUGACGUTT | A30381 | 58 | ACGUCAUUAUUCUUAGACATT |
| A30382 | 59 | AACCCUACACUCGGAAUUCTT | A30383 | 60 | GAAUUCCGAGUGUAGGGUUTT |
| A30384 | 61 | AAAAGAGGGUGUUCUCUAUTT | A30385 | 62 | AUAGAGAACACCCUCUUUUTT |
| A30386 | 63 | GGGUGUUCUCUAUGUAGGCTT | A30387 | 64 | GCCUACAUAGAGAACACCCTT |
| A30388 | 65 | UCUCUAUGUAGGCUCCAAATT | A30389 | 66 | UUUGGAGCCUACAUAGAGATT |
| A30390 | 67 | GAAGGGUAUCAAGACUACGTT | A30391 | 68 | CGUAGUCUUGAUACCCUUCTT |
| A30392 | 69 | AAGGGUAUCAAGACUACGATT | A30393 | 70 | UCGUAGUCUUGAUACCCUUTT |
| A30394 | 71 | GGGUAUCAAGACUACGAACTT | A30395 | 72 | GUUCGUAGUCUUGAUACCCTT |
| A30396 | 73 | GAAGCCUAAGAAAUAUCUUTT | A30397 | 74 | AAGAUAUUUCUUAGGCUUCTT |
| A30398 | 75 | UUUACAGUGUAUCUCGAAGTT | A30399 | 76 | CUUCGAGAUACACUGUAAATT |
| A30400 | 77 | UACAGUGUAUCUCGAAGUCTT | A30401 | 78 | GACUUCGAGAUACACUGUATT |
| A30402 | 79 | CACUCAGCAUUUCGGUGCUTT | A30403 | 80 | AGCACCGAAAUGCUGAGUGTT |
| A30404 | 81 | UGGCUUCAAUCUACGAUGUTT | A30405 | 82 | ACAUCGUAGAUUGAAGCCATT |
| A30406 | 83 | CUUCAAUCUACGAUGUUAATT | A30407 | 84 | UUAACAUCGUAGAUUGAAGTT |
| A30408 | 85 | AAUCUACGAUGUUAAAACATT | A30409 | 86 | UGUUUUAACAUCGUAGAUUTT |
| A30410 | 87 | AACACCUAAGUGACUACCATT | A30411 | 88 | UGGUAGUCACUUAGGUGUUTT |
| A30412 | 89 | UAAGUGACUACCACUUAUUTT | A30413 | 90 | AAUAAGUGGUAGUCACUUATT |
| A30414 | 91 | UGUUCAGAAGUUGUUAGUGTT | A30415 | 92 | CACUAACAACUUCUGAACATT |
| A30416 | 93 | AAGUUGUUAGUGAUUUGCUTT | A30417 | 94 | AGCAAAUCACUAACAACUUTT |
| A30418 | 95 | AUAAGAUUUUUAGGUGUCUTT | A30419 | 96 | AGACACCUAAAAAUCUUAUTT |
| A30420 | 97 | AAUGACGUAUUGUGAAAUUTT | A30421 | 98 | AAUUUCACAAUACGUCAUUTT |
| A30422 | 99 | GCAUGAAACUAUGCACCUATT | A30423 | 100 | UAGGUGCAUAGUUUCAUGCTT |
| A30424 | 101 | UAUGCACCUAUAAAUACUATT | A30425 | 102 | UAGUAUUUAUAGGUGCAUATT |
| A30426 | 103 | UACCAUUUUGCGAUGUGUUTT | A30427 | 104 | AACACAUCGCAAAAUGGUATT |
| A30428 | 105 | AUUUUUAUCCCAUCUCACUTT | A30429 | 106 | AGUGAGAUGGGAUAAAAAUTT |
| A30430 | 107 | UUAUCCCAUCUCACUUUAATT | A30431 | 108 | UUAAAGUGAGAUGGGAUAATT |
| A30432 | 109 | UAACCCUACACUCGGAAUUTT | A30433 | 110 | AAUUCCGAGUGUAGGGUUATT |
| A30434 | 111 | AGGGUGUUCUCUAUGUAGGTT | A30435 | 112 | CCUACAUAGAGAACACCCUTT |
| A30436 | 113 | CUCUAUGUAGGCUCCAAAATT | A30437 | 114 | UUUUGGAGCCUACAUAGAGTT |
| A30438 | 115 | AACCAAACAGGGUGUGGCATT | A30439 | 116 | UGCCACACCCUGUUUGGUUTT |
| A30440 | 117 | GAGGGUGUUCUCUAUGUAGTT | A30441 | 118 | CUACAUAGAGAACACCCUCTT |
| A30442 | 119 | UUCUCUAUGUAGGCUCCAATT | A30443 | 120 | UUGGAGCCUACAUAGAGAATT |
| A30444 | 121 | GUGACAAAUGUUGGAGGAGTT | A30445 | 122 | CUCCUCCAACAUUUGUCACTT |
| A30446 | 123 | GUCAAAAAGGACCAGUUGGTT | A30447 | 124 | CCAACUGGUCCUUUUUGACTT |
| A30448 | 125 | UCAAGACUACGAACCUGAATT | A30449 | 126 | UUCAGGUUCGUAGUCUUGATT |
| A30450 | 127 | CAGCAUUUCGGUGCUUCCCTT | A30451 | 128 | GGGAAGCACCGAAAUGCUGTT |
| A30452 | 129 | GUUGUUCAGAAGUUGUUAGTT | A30453 | 130 | CUAACAACUUCUGAACAACTT |
| A30454 | 131 | GAAGUUGUUAGUGAUUUGCTT | A30455 | 132 | GCAAAUCACUAACAACUUCTT |
| A30456 | 133 | AGUUGUUAGUGAUUUGCUATT | A30457 | 134 | UAGCAAAUCACUAACAACUTT |
| A30458 | 135 | UUAGUGAUUUGCUAUCAUATT | A30459 | 136 | UAUGAUAGCAAAUCACUAATT |
| A30460 | 137 | GUGAUUUGCUAUCAUAUAUTT | A30461 | 138 | AUAUAUGAUAGCAAAUCACTT |
| A30462 | 139 | UAAUGACGUAUUGUGAAAUTT | A30463 | 140 | AUUUCACAAUACGUCAUUATT |
| A30464 | 141 | CUAUGCACCUAUAAAUACUTT | A30465 | 142 | AGUAUUUAUAGGUGCAUAGTT |
| A30466 | 143 | AUUAGCCAUGGAUGUAUUCTT | A30467 | 144 | GAAUACAUCCAUGGCUAAUTT |
| A30468 | 145 | UUAGCCAUGGAUGUAUUCATT | A30469 | 146 | UGAAUACAUCCAUGGCUAATT |
| A30470 | 147 | GAAAGGACUUUCAAAGGCCTT | A30471 | 148 | GGCCUUUGAAAGUCCUUUCTT |
| A30472 | 149 | CAAAAGAGGGUGUUCUCUATT | A30473 | 150 | UAGAGAACACCCUCUUUUGTT |
| A30474 | 151 | AGAGGGUGUUCUCUAUGUATT | A30475 | 152 | UACAUAGAGAACACCCUCUTT |
| A30476 | 153 | GGUGUUCUCUAUGUAGGCUTT | A30477 | 154 | AGCCUACAUAGAGAACACCTT |
| A30478 | 155 | GUGUUCUCUAUGUAGGCUCTT | A30479 | 156 | GAGCCUACAUAGAGAACACTT |
| A30480 | 157 | UGUUCUCUAUGUAGGCUCCTT | A30481 | 158 | GGAGCCUACAUAGAGAACATT |
| A30482 | 159 | GGAGCAGUGGUGACGGGUGTT | A30483 | 160 | CACCCGUCACCACUGCUCCTT |
| A30484 | 161 | CAGUGGUGACGGGUGUGACTT | A30485 | 162 | GUCACACCCGUCACCACUGTT |
| A30486 | 163 | UAUCAAGACUACGAACCUGTT | A30487 | 164 | CAGGUUCGUAGUCUUGAUATT |
| A30488 | 165 | CUCCCAGUUUCUUGAGAUCTT | A30489 | 166 | GAUCUCAAGAAACUGGGAGTT |
| A30490 | 167 | GACAGAUGUUCCAUCCUGUTT | A30491 | 168 | ACAGGAUGGAACAUCUGUCTT |
| A30492 | 169 | AAUGUGCCCAGUCAUGACATT | A30493 | 170 | UGUCAUGACUGGGCACAUUTT |
| A30494 | 171 | UGCCCAGUCAUGACAUUUCTT | A30495 | 172 | GAAAUGUCAUGACUGGGCATT |
| A30496 | 173 | CAGUCAUGACAUUUCUCAATT | A30497 | 174 | UUGAGAAAUGUCAUGACUGTT |
| A30498 | 175 | AAGUUUUUACAGUGUAUCUTT | A30499 | 176 | AGAUACACUGUAAAAACUUTT |
| A30500 | 177 | CAGUGUAUCUCGAAGUCUUTT | A30501 | 178 | AAGACUUCGAGAUACACUGTT |
| A30502 | 179 | GUAUCUCGAAGUCUUCCAUTT | A30503 | 180 | AUGGAAGACUUCGAGAUACTT |
| A30504 | 181 | CAGCAGUGAUUGAAGUAUCTT | A30505 | 182 | GAUACUUCAAUCACUGCUGTT |
| A30506 | 183 | CCACUCAGCAUUUCGGUGCTT | A30507 | 184 | GCACCGAAAUGCUGAGUGGTT |
| A30508 | 185 | UCAGCAUUUCGGUGCUUCCTT | A30509 | 186 | GGAAGCACCGAAAUGCUGATT |
| A30510 | 187 | AAGUGAAUACAUGGUAGCATT | A30511 | 188 | UGCUACCAUGUAUUCACUUTT |
| A30512 | 189 | GAAUACAUGGUAGCAGGGUTT | A30513 | 190 | ACCCUGCUACCAUGUAUUCTT |
| A30514 | 191 | UGGUAGCAGGGUCUUUGUGTT | A30515 | 192 | CACAAAGACCCUGCUACCATT |
| A30516 | 193 | UUUUGUGGCUUCAAUCUACTT | A30517 | 194 | GUAGAUUGAAGCCACAAAATT |
| A30518 | 195 | UUUGUGGCUUCAAUCUACGTT | A30519 | 196 | CGUAGAUUGAAGCCACAAATT |
| A30520 | 197 | AAUUAAAAACACCUAAGUGTT | A30521 | 198 | CACUUAGGUGUUUUUAAUUTT |
| A30522 | 199 | AUUAAAAACACCUAAGUGATT | A30523 | 200 | UCACUUAGGUGUUUUUAAUTT |
| A30524 | 201 | AAACACCUAAGUGACUACCTT | A30525 | 202 | GGUAGUCACUUAGGUGUUUTT |
| A30526 | 203 | GACUACCACUUAUUUCUAATT | A30527 | 204 | UUAGAAAUAAGUGGUAGUCTT |
| A30528 | 205 | UUAUUUCUAAAUCCUCACUTT | A30529 | 206 | AGUGAGGAUUUAGAAAUAATT |
| A30530 | 207 | CAGAAGUUGUUAGUGAUUUTT | A30531 | 208 | AAAUCACUAACAACUUCUGTT |
| A30532 | 209 | UUAUAAGAUUUUUAGGUGUTT | A30533 | 210 | ACACCUAAAAAUCUUAUAATT |
| A30534 | 211 | UAUAAGAUUUUUAGGUGUCTT | A30535 | 212 | GACACCUAAAAAUCUUAUATT |
| A30536 | 213 | UAAGAAUAAUGACGUAUUGTT | A30537 | 214 | CAAUACGUCAUUAUUCUUATT |
| A30538 | 215 | AACUAUGCACCUAUAAAUATT | A30539 | 216 | UAUUUAUAGGUGCAUAGUUTT |
| A30540 | 217 | AAAUUUUACCAUUUUGCGATT | A30541 | 218 | UCGCAAAAUGGUAAAAUUUTT |
| A30542 | 219 | AAUUUUACCAUUUUGCGAUTT | A30543 | 220 | AUCGCAAAAUGGUAAAAUUTT |
| A30544 | 221 | UUUUACCAUUUUGCGAUGUTT | A30545 | 222 | ACAUCGCAAAAUGGUAAAATT |
| A30546 | 223 | UUGCGAUGUGUUUUAUUCATT | A30547 | 224 | UGAAUAAAACACAUCGCAATT |
| A30548 | 225 | UGCGAUGUGUUUUAUUCACTT | A30549 | 226 | GUGAAUAAAACACAUCGCATT |
| A30550 | 227 | CGUUAUCUCAUUGCAAAAATT | A30551 | 228 | UUUUUGCAAUGAGAUAACGTT |
| A30552 | 229 | UAUCCCAUCUCACUUUAAUTT | A30553 | 230 | AUUAAAGUGAGAUGGGAUATT |
| A30554 | 231 | AUCCCAUCUCACUUUAAUATT | A30555 | 232 | UAUUAAAGUGAGAUGGGAUTT |
| A30556 | 233 | CAUCUCACUUUAAUAAUAATT | A30557 | 234 | UUAUUAUUAAAGUGAGAUGTT |
| A30558 | 235 | AAAUCAUGCUUAUAAGCAATT | A30559 | 236 | UUGCUUAUAAGCAUGAUUUTT |
| A30560 | 237 | AUGCUUAUAAGCAACAUGATT | A30561 | 238 | UCAUGUUGCUUAUAAGCAUTT |
| A30562 | 239 | AGAACUGACACAAAGGACATT | A30563 | 240 | UGUCCUUUGUGUCAGUUCUTT |
| A30564 | 241 | AUAAAGUUAUUAAUAGCCATT | A30565 | 242 | UGGCUAUUAAUAACUUUAUTT |
| A30566 | 243 | AAAGUUAUUAAUAGCCAUUTT | A30567 | 244 | AAUGGCUAUUAAUAACUUUTT |
| A30568 | 245 | GGAUGUAUUCAUGAAAGGATT | A30569 | 246 | UCCUUUCAUGAAUACAUCCTT |
| A30570 | 247 | GUUCUCUAUGUAGGCUCCATT | A30571 | 248 | UGGAGCCUACAUAGAGAACTT |
| A30572 | 249 | GUUCCAUCCUGUACAAGUGTT | A30573 | 250 | CACUUGUACAGGAUGGAACTT |
| A30574 | 251 | CUAAAUCCUCACUAUUUUUTT | A30575 | 252 | AAAAAUAGUGAGGAUUUAGTT |
| A30576 | 253 | CUAUGUAGGCUCCAAAACCTT | A30577 | 254 | GGUUUUGGAGCCUACAUAGTT |
| A30578 | 255 | GGUGCAUGGUGUGGCAACATT | A30579 | 256 | UGUUGCCACACCAUGCACCTT |
| A30580 | 257 | UGAGAUCUGCUGACAGAUGTT | A30581 | 258 | CAUCUGUCAGCAGAUCUCATI |
| A30582 | 259 | CCCACUCAGCAUUUCGGUGTT | A30583 | 260 | CACCGAAAUGCUGAGUGGGTT |
| A30584 | 261 | CAUGGUAGCAGGGUCUUUGTT | A30585 | 262 | CAAAGACCCUGCUACCAUGTT |
| A30586 | 263 | AGAAUUAAAAUAAAACGUUTT | A30587 | 264 | AACGUUUUAUUUUAAUUCUTT |
| A30588 | 265 | AUCUCGAAGUCUUCCAUCATT | A30589 | 266 | UGAUGGAAGACUUCGAGAUTT |
| A30590 | 267 | GUGAGCAUGAAACUAUGCATT | A30591 | 268 | UGCAUAGUUUCAUGCUCACTT |

TABLE 3

siRNAs targeting alpha-synuclein.
Sequences have no overhangs.

| 23mer pos. | | | | |
|---|---|---|---|---|
| in | SEQ ID | Sense strand | SEQ ID | Antisense strand |
| NM_000345 | NO: | sequence (5′-3′) | NO: | sequence (5′-3′) |
| 703-721 | 269 | GUGGCUUCAAUCUACGAUG | 270 | CAUCGUAGAUUGAAGCCAC |
| 569-587 | 271 | UUUUUACAGUGUAUCUCGA | 272 | UCGAGAUACACUGUAAAAA |
| 442-460 | 273 | GUAUCAAGACUACGAACCU | 274 | AGGUUCGUAGUCUUGAUAC |
| 570-588 | 275 | UUUUACAGUGUAUCUCGAA | 276 | UUCGAGAUACACUGUAAAA |
| 1190-1208 | 277 | ACAUUAACCCUACACUCGG | 278 | CCGAGUGUAGGGUUAAUGU |
| 702-720 | 279 | UGUGGCUUCAAUCUACGAU | 280 | AUCGUAGAUUGAAGCCACA |
| 631-649 | 281 | CUCAGCAUUUCGGUGCUUC | 282 | GAAGCACCGAAAUGCUGAG |
| 1193-1211 | 283 | UUAACCCUACACUCGGAAU | 284 | AUUCCGAGUGUAGGGUUAA |
| 572-590 | 285 | UUACAGUGUAUCUCGAAGU | 286 | ACUUCGAGAUACACUGUAA |
| 574-592 | 287 | ACAGUGUAUCUCGAAGUCU | 288 | AGACUUCGAGAUACACUGU |
| 706-724 | 289 | GCUUCAAUCUACGAUGUUA | 290 | UAACAUCGUAGAUUGAAGC |
| 1192-1210 | 291 | AUUAACCCUACACUCGGAA | 292 | UUCCGAGUGUAGGGUUAAU |
| 848-866 | 293 | UUAAUGAUACUGUCUAAGA | 294 | UCUUAGACAGUAUCAUUAA |
| 444-462 | 295 | AUCAAGACUACGAACCUGA | 296 | UCAGGUUCGUAGUCUUGAU |
| 661-679 | 297 | AGUGAAUACAUGGUAGCAG | 298 | CUGCUACCAUGUAUUCACU |
| 665-683 | 299 | AAUACAUGGUAGCAGGGUC | 300 | GACCCUGCUACCAUGUAUU |
| 742-760 | 301 | CUAAGUGACUACCACUUAU | 302 | AUAAGUGGUAGUCACUUAG |
| 919-937 | 303 | AGCAUGAAACUAUGCACCU | 304 | AGGUGCAUAGUUUCAUGCU |
| 701-719 | 305 | UUGUGGCUUCAAUCUACGA | 306 | UCGUAGAUUGAAGCCACAA |
| 705-723 | 307 | GGCUUCAAUCUACGAUGUU | 308 | AACAUCGUAGAUUGAAGCC |
| 867-885 | 309 | AUAAUGACGUAUUGUGAAA | 310 | UUUCACAAUACGUCAUUAU |
| 807-825 | 311 | AGUGAUUUGCUAUCAUAUA | 312 | UAUAUGAUAGCAAAUCACU |
| 710-728 | 313 | CAAUCUACGAUGUUAAAAC | 314 | GUUUUAACAUCGUAGAUUG |
| 873-891 | 315 | ACGUAUUGUGAAAUUUGUU | 316 | AACAAAUUUCACAAUACGU |
| 872-890 | 317 | GACGUAUUGUGAAAUUUGU | 318 | ACAAAUUUCACAAUACGUC |
| 260-278 | 319 | ACGGGUGUGACAGCAGUAG | 320 | CUACUGCUGUCACACCCGU |
| 441-459 | 321 | GGUAUCAAGACUACGAACC | 322 | GGUUCGUAGUCUUGAUACC |
| 858-876 | 323 | UGUCUAAGAAUAAUGACGU | 324 | ACGUCAUUAUUCUUAGACA |
| 1195-1213 | 325 | AACCCUACACUCGGAAUUC | 326 | GAAUUCCGAGUGUAGGGUU |
| 145-163 | 327 | AAAAGAGGGUGUUCUCUAU | 328 | AUAGAGAACACCCUCUUUU |
| 151-169 | 329 | GGGUGUUCUCUAUGUAGGC | 330 | GCCUACAUAGAGAACACCC |
| 157-175 | 331 | UCUCUAUGUAGGCUCCAAA | 332 | UUUGGAGCCUACAUAGAGA |
| 437-455 | 333 | GAAGGGUAUCAAGACUACG | 334 | CGUAGUCUUGAUACCCUUC |
| 438-456 | 335 | AAGGGUAUCAAGACUACGA | 336 | UCGUAGUCUUGAUACCCUU |
| 440-458 | 337 | GGGUAUCAAGACUACGAAC | 338 | GUUCGUAGUCUUGAUACCC |
| 461-479 | 339 | GAAGCCUAAGAAAUAUCUU | 340 | AAGAUAUUUCUUAGGCUUC |
| 571-589 | 341 | UUUACAGUGUAUCUCGAAG | 342 | CUUCGAGAUACACUGUAAA |
| 573-591 | 343 | UACAGUGUAUCUCGAAGUC | 344 | GACUUCGAGAUACACUGUA |
| 629-647 | 345 | CACUCAGCAUUUCGGUGCU | 346 | AGCACCGAAAUGCUGAGUG |
| 704-722 | 347 | UGGCUUCAAUCUACGAUGU | 348 | ACAUCGUAGAUUGAAGCCA |
| 707-725 | 349 | CUUCAAUCUACGAUGUUAA | 350 | UUAACAUCGUAGAUUGAAG |
| 711-729 | 351 | AAUCUACGAUGUUAAAACA | 352 | UGUUUUAACAUCGUAGAUU |
| 737-755 | 353 | AACACCUAAGUGACUACCA | 354 | UGGUAGUCACUUAGGUGUU |
| 743-761 | 355 | UAAGUGACUACCACUUAUU | 356 | AAUAAGUGGUAGUCACUUA |
| 792-810 | 357 | UGUUCAGAAGUUGUUAGUG | 358 | CACUAACAACUUCUGAACA |
| 799-817 | 359 | AAGUUGUUAGUGAUUUGCU | 360 | AGCAAAUCACUAACAACUU |
| 828-846 | 361 | AUAAGAUUUUUAGGUGUCU | 362 | AGACACCUAAAAAUCUUAU |
| 869-887 | 363 | AAUGACGUAUUGUGAAAUU | 364 | AAUUUCACAAUACGUCAUU |
| 920-938 | 365 | GCAUGAAACUAUGCACCUA | 366 | UAGGUGCAUAGUUUCAUGC |
| 929-947 | 367 | UAUGCACCUAUAAAUACUA | 368 | UAGUAUUUAUAGGUGCAUA |
| 960-978 | 369 | UACCAUUUUGCGAUGUGUU | 370 | AACACAUCGCAAAAUGGUA |
| 1049-1067 | 371 | AUUUUUAUCCCAUCUCACU | 372 | AGUGAGAUGGGAUAAAAAU |
| 1053-1071 | 373 | UUAUCCCAUCUCACUUUAA | 374 | UUAAAGUGAGAUGGGAUAA |
| 1194-1212 | 375 | UAACCCUACACUCGGAAUU | 376 | AAUUCCGAGUGUAGGGUUA |
| 150-168 | 377 | AGGGUGUUCUCUAUGUAGG | 378 | CCUACAUAGAGAACACCCU |
| 158-176 | 379 | CUCUAUGUAGGCUCCAAAA | 380 | UUUUGGAGCCUACAUAGAG |
| 109-127 | 381 | AACCAAACAGGGUGUGGCA | 382 | UGCCACACCCUGUUUGGUU |
| 149-167 | 383 | GAGGGUGUUCUCUAUGUAG | 384 | CUACAUAGAGAACACCCUC |
| 156-174 | 385 | UUCUCUAUGUAGGCUCCAA | 386 | UUGGAGCCUACAUAGAGAA |
| 233-251 | 387 | GUGACAAAUGUUGGAGGAG | 388 | CUCCUCCAACAUUUGUCAC |
| 329-347 | 389 | GUCAAAAAGGACCAGUUGG | 390 | CCAACUGGUCCUUUUUGAC |
| 445-463 | 391 | UCAAGACUACGAACCUGAA | 392 | UUCAGGUUCGUAGUCUUGA |
| 633-651 | 393 | CAGCAUUUCGGUGCUUCCC | 394 | GGGAAGCACCGAAAUGCUG |
| 790-808 | 395 | GUUGUUCAGAAGUUGUUAG | 396 | CUAACAACUUCUGAACAAC |
| 798-816 | 397 | GAAGUUGUUAGUGAUUUGC | 398 | GCAAAUCACUAACAACUUC |
| 800-818 | 399 | AGUUGUUAGUGAUUUGCUA | 400 | UAGCAAAUCACUAACAACU |
| 805-823 | 401 | UUAGUGAUUUGCUAUCAUA | 402 | UAUGAUAGCAAAUCACUAA |
| 808-826 | 403 | GUGAUUUGCUAUCAUAUAU | 404 | AUAUAUGAUAGCAAAUCAC |
| 868-886 | 405 | UAAUGACGUAUUGUGAAAU | 406 | AUUUCACAAUACGUCAUUA |
| 928-946 | 407 | CUAUGCACCUAUAAAUACU | 408 | AGUAUUUAUAGGUGCAUAG |
| 40-58 | 409 | AUUAGCCAUGGAUGUAUUC | 410 | GAAUACAUCCAUGGCUAAU |
| 41-59 | 411 | UUAGCCAUGGAUGUAUUCA | 412 | UGAAUACAUCCAUGGCUAA |
| 61-79 | 413 | GAAAGGACUUUCAAAGGCC | 414 | GGCCUUUGAAAGUCCUUUC |
| 144-162 | 415 | CAAAAGAGGGUGUUCUCUA | 416 | UAGAGAACACCCUCUUUUG |
| 148-166 | 417 | AGAGGGUGUUCUCUAUGUA | 418 | UACAUAGAGAACACCCUCU |
| 152-170 | 419 | GGUGUUCUCUAUGUAGGCU | 420 | AGCCUACAUAGAGAACACC |
| 153-171 | 421 | GUGUUCUCUAUGUAGGCUC | 422 | GAGCCUACAUAGAGAACAC |
| 154-172 | 423 | UGUUCUCUAUGUAGGCUCC | 424 | GGAGCCUACAUAGAGAACA |
| 248-266 | 425 | GGAGCAGUGGUGACGGGUG | 426 | CACCCGUCACCACUGCUCC |
| 252-270 | 427 | CAGUGGUGACGGGUGUGAC | 428 | GUCACACCCGUCACCACUG |
| 443-461 | 429 | UAUCAAGACUACGAACCUG | 430 | CAGGUUCGUAGUCUUGAUA |
| 482-500 | 431 | CUCCCAGUUUCUUGAGAUC | 432 | GAUCUCAAGAAACUGGGAG |
| 505-523 | 433 | GACAGAUGUUCCAUCCUGU | 434 | ACAGGAUGGAACAUCUGUC |
| 540-558 | 435 | AAUGUGCCCAGUCAUGACA | 436 | UGUCAUGACUGGGCACAUU |
| 544-562 | 437 | UGCCCAGUCAUGACAUUUC | 438 | GAAAUGUCAUGACUGGGCA |
| 548-566 | 439 | CAGUCAUGACAUUUCUCAA | 440 | UUGAGAAAUGUCAUGACUG |
| 566-584 | 441 | AAGUUUUUACAGUGUAUCU | 442 | AGAUACACUGUAAAAACUU |
| 575-593 | 443 | CAGUGUAUCUCGAAGUCUU | 444 | AAGACUUCGAGAUACACUG |
| 579-597 | 445 | GUAUCUCGAAGUCUUCCAU | 446 | AUGGAAGACUUCGAGAUAC |
| 598-616 | 447 | CAGCAGUGAUUGAAGUAUC | 448 | GAUACUUCAAUCACUGCUG |
| 628-646 | 449 | CCACUCAGCAUUUCGGUGC | 450 | GCACCGAAAUGCUGAGUGG |
| 632-650 | 451 | UCAGCAUUUCGGUGCUUCC | 452 | GGAAGCACCGAAAUGCUGA |
| 660-678 | 453 | AAGUGAAUACAUGGUAGCA | 454 | UGCUACCAUGUAUUCACUU |
| 664-682 | 455 | GAAUACAUGGUAGCAGGGU | 456 | ACCCUGCUACCAUGUAUUC |
| 671-689 | 457 | UGGUAGCAGGGUCUUUGUG | 458 | CACAAAGACCCUGCUACCA |
| 699-717 | 459 | UUUUGUGGCUUCAAUCUAC | 460 | GUAGAUUGAAGCCACAAAA |
| 700-718 | 461 | UUUGUGGCUUCAAUCUACG | 462 | CGUAGAUUGAAGCCACAAA |
| 730-748 | 463 | AAUUAAAAACACCUAAGUG | 464 | CACUUAGGUGUUUUUAAUU |
| 731-749 | 465 | AUUAAAAACACCUAAGUGA | 466 | UCACUUAGGUGUUUUUAAU |
| 736-754 | 467 | AAACACCUAAGUGACUACC | 468 | GGUAGUCACUUAGGUGUUU |
| 748-766 | 469 | GACUACCACUUAUUUCUAA | 470 | UUAGAAAUAAGUGGUAGUC |
| 757-775 | 471 | UUAUUUCUAAAUCCUCACU | 472 | AGUGAGGAUUUAGAAAUAA |
| 796-814 | 473 | CAGAAGUUGUUAGUGAUUU | 474 | AAAUCACUAACAACUUCUG |
| 826-844 | 475 | UUAUAAGAUUUUUAGGUGU | 476 | ACACCUAAAAAUCUUAUAA |
| 827-845 | 477 | UAUAAGAUUUUUAGGUGUC | 478 | GACACCUAAAAAUCUUAUA |
| 862-880 | 479 | UAAGAAUAAUGACGUAUUG | 480 | CAAUACGUCAUUAUUCUUA |
| 926-944 | 481 | AACUAUGCACCUAUAAAUA | 482 | UAUUUAUAGGUGCAUAGUU |
| 954-972 | 483 | AAAUUUUACCAUUUUGCGA | 484 | UCGCAAAAUGGUAAAAUUU |
| 955-973 | 485 | AAUUUUACCAUUUUGCGAU | 486 | AUCGCAAAAUGGUAAAAUU |
| 957-975 | 487 | UUUUACCAUUUUGCGAUGU | 488 | ACAUCGCAAAAUGGUAAAA |
| 967-985 | 489 | UUGCGAUGUGUUUUAUUCA | 490 | UGAAUAAAACACAUCGCAA |
| 968-986 | 491 | UGCGAUGUGUUUUAUUCAC | 492 | GUGAAUAAAACACAUCGCA |
| 1024-1042 | 493 | CGUUAUCUCAUUGCAAAAA | 494 | UUUUUGCAAUGAGAUAACG |
| 1054-1072 | 495 | UAUCCCAUCUCACUUUAAU | 496 | AUUAAAGUGAGAUGGGAUA |
| 1055-1073 | 497 | AUCCCAUCUCACUUUAAUA | 498 | UAUUAAAGUGAGAUGGGAU |
| 1059-1077 | 499 | CAUCUCACUUUAAUAAUAA | 500 | UUAUUAUUAAAGUGAGAUG |
| 1078-1096 | 501 | AAAUCAUGCUUAUAAGCAA | 502 | UUGCUUAUAAGCAUGAUUU |
| 1083-1101 | 503 | AUGCUUAUAAGCAACAUGA | 504 | UCAUGUUGCUUAUAAGCAU |
| 1106-1124 | 505 | AGAACUGACACAAAGGACA | 506 | UGUCCUUUGUGUCAGUUCU |
| 1130-1148 | 507 | AUAAAGUUAUUAAUAGCCA | 508 | UGGCUAUUAAUAACUUUAU |
| 1132-1150 | 509 | AAAGUUAUUAAUAGCCAUU | 510 | AAUGGCUAUUAAUAACUUU |
| 49-67 | 511 | GGAUGUAUUCAUGAAAGGA | 512 | UCCUUUCAUGAAUACAUCC |
| 155-173 | 513 | GUUCUCUAUGUAGGCUCCA | 514 | UGGAGCCUACAUAGAGAAC |
| 512-530 | 515 | GUUCCAUCCUGUACAAGUG | 516 | CACUUGUACAGGAUGGAAC |
| 763-781 | 517 | CUAAAUCCUCACUAUUUUU | 518 | AAAAAUAGUGAGGAUUUAG |
| 160-178 | 519 | CUAUGUAGGCUCCAAAACC | 520 | GGUUUUGGAGCCUACAUAG |
| 190-208 | 521 | GGUGCAUGGUGUGGCAACA | 522 | UGUUGCCACACCAUGCACC |
| 494-512 | 523 | UGAGAUCUGCUGACAGAUG | 524 | CAUCUGUCAGCAGAUCUCA |
| 627-645 | 525 | CCCACUCAGCAUUUCGGUG | 526 | CACCGAAAUGCUGAGUGGG |
| 669-687 | 527 | CAUGGUAGCAGGGUCUUUG | 528 | CAAAGACCCUGCUACCAUG |
| 1009-1027 | 529 | AGAAUUAAAAUAAAACGUU | 530 | AACGUUUUAUUUUAAUUCU |
| 581-599 | 531 | AUCUCGAAGUCUUCCAUCA | 532 | UGAUGGAAGACUUCGAGAU |
| 916-934 | 533 | GUGAGCAUGAAACUAUGCA | 534 | UGCAUAGUUUCAUGCUCAC |

TABLE 4

| siRNAs targeting alpha-synuclein. | | | | |
|---|---|---|---|---|
| Sequences have dinucleotide overhangs | | | | |
| (N = A, C, G, U, or T). | | | | |
| 23mer pos. | SEQ | | SEQ | |
| in | ID | Sense strand | ID | Antisense strand |
| NM_000345 | NO: | sequence (5′-3′) | NO: | sequence (5′-3′) |
| 703-721 | 535 | GUGGCUUCAAUCUACGAUGNN | 536 | CAUCGUAGAUUGAAGCCACNN |
| 569-587 | 537 | UUUUUACAGUGUAUCUCGANN | 538 | UCGAGAUACACUGUAAAAANN |
| 442-460 | 539 | GUAUCAAGACUACGAACCUNN | 540 | AGGUUCGUAGUCUUGAUACNN |
| 570-588 | 541 | UUUUACAGUGUAUCUCGAANN | 542 | UUCGAGAUACACUGUAAAANN |
| 1190-1208 | 543 | ACAUUAACCCUACACUCGGNN | 544 | CCGAGUGUAGGGUUAAUGUNN |
| 702-720 | 545 | UGUGGCUUCAAUCUACGAUNN | 546 | AUCGUAGAUUGAAGCCACANN |
| 631-649 | 547 | CUCAGCAUUUCGGUGCUUCNN | 548 | GAAGCACCGAAAUGCUGAGNN |
| 1193-1211 | 549 | UUAACCCUACACUCGGAAUNN | 550 | AUUCCGAGUGUAGGGUUAANN |
| 572-590 | 551 | UUACAGUGUAUCUCGAAGUNN | 552 | ACUUCGAGAUACACUGUAANN |
| 574-592 | 553 | ACAGUGUAUCUCGAAGUCUNN | 554 | AGACUUCGAGAUACACUGUNN |
| 706-724 | 555 | GCUUCAAUCUACGAUGUUANN | 556 | UAACAUCGUAGAUUGAAGCNN |
| 1192-1210 | 557 | AUUAACCCUACACUCGGAANN | 558 | UUCCGAGUGUAGGGUUAAUNN |
| 848-866 | 559 | UUAAUGAUACUGUCUAAGANN | 560 | UCUUAGACAGUAUCAUUAANN |
| 444-462 | 561 | AUCAAGACUACGAACCUGANN | 562 | UCAGGUUCGUAGUCUUGAUNN |
| 661-679 | 563 | AGUGAAUACAUGGUAGCAGNN | 564 | CUGCUACCAUGUAUUCACUNN |
| 665-683 | 565 | AAUACAUGGUAGCAGGGUCNN | 566 | GACCCUGCUACCAUGUAUUNN |
| 742-760 | 567 | CUAAGUGACUACCACUUAUNN | 568 | AUAAGUGGUAGUCACUUAGNN |
| 919-937 | 569 | AGCAUGAAACUAUGCACCUNN | 570 | AGGUGCAUAGUUUCAUGCUNN |
| 701-719 | 571 | UUGUGGCUUCAAUCUACGANN | 572 | UCGUAGAUUGAAGCCACAANN |
| 705-723 | 573 | GGCUUCAAUCUACGAUGUUNN | 574 | AACAUCGUAGAUUGAAGCCNN |
| 867-885 | 575 | AUAAUGACGUAUUGUGAAANN | 576 | UUUCACAAUACGUCAUUAUNN |
| 807-825 | 577 | AGUGAUUUGCUAUCAUAUANN | 578 | UAUAUGAUAGCAAAUCACUNN |
| 710-728 | 579 | CAAUCUACGAUGUUAAAACNN | 580 | GUUUUAACAUCGUAGAUUGNN |
| 873-891 | 581 | ACGUAUUGUGAAAUUUGUUNN | 582 | AACAAAUUUCACAAUACGUNN |
| 872-890 | 583 | GACGUAUUGUGAAAUUUGUNN | 584 | ACAAAUUUCACAAUACGUCNN |
| 260-278 | 585 | ACGGGUGUGACAGCAGUAGNN | 586 | CUACUGCUGUCACACCCGUNN |
| 441-459 | 587 | GGUAUCAAGACUACGAACCNN | 588 | GGUUCGUAGUCUUGAUACCNN |
| 858-876 | 589 | UGUCUAAGAAUAAUGACGUNN | 590 | ACGUCAUUAUUCUUAGACANN |
| 1195-1213 | 591 | AACCCUACACUCGGAAUUCNN | 592 | GAAUUCCGAGUGUAGGGUUNN |
| 145-163 | 593 | AAAAGAGGGUGUUCUCUAUNN | 594 | AUAGAGAACACCCUCUUUUNN |
| 151-169 | 595 | GGGUGUUCUCUAUGUAGGCNN | 596 | GCCUACAUAGAGAACACCCNN |
| 157-175 | 597 | UCUCUAUGUAGGCUCCAAANN | 598 | UUUGGAGCCUACAUAGAGANN |
| 437-455 | 599 | GAAGGGUAUCAAGACUACGNN | 600 | CGUAGUCUUGAUACCCUUCNN |
| 438-456 | 601 | AAGGGUAUCAAGACUACGANN | 602 | UCGUAGUCUUGAUACCCUUNN |
| 440-458 | 603 | GGGUAUCAAGACUACGAACNN | 604 | GUUCGUAGUCUUGAUACCCNN |
| 461-479 | 605 | GAAGCCUAAGAAAUAUCUUNN | 606 | AAGAUAUUUCUUAGGCUUCNN |
| 571-589 | 607 | UUUACAGUGUAUCUCGAAGNN | 608 | CUUCGAGAUACACUGUAAANN |
| 573-591 | 609 | UACAGUGUAUCUCGAAGUCNN | 610 | GACUUCGAGAUACACUGUANN |
| 629-647 | 611 | CACUCAGCAUUUCGGUGCUNN | 612 | AGCACCGAAAUGCUGAGUGNN |
| 704-722 | 613 | UGGCUUCAAUCUACGAUGUNN | 614 | ACAUCGUAGAUUGAAGCCANN |
| 707-725 | 615 | CUUCAAUCUACGAUGUUAANN | 616 | UUAACAUCGUAGAUUGAAGNN |
| 711-729 | 617 | AAUCUACGAUGUUAAAACANN | 618 | UGUUUUAACAUCGUAGAUUNN |
| 737-755 | 619 | AACACCUAAGUGACUACCANN | 620 | UGGUAGUCACUUAGGUGUUNN |
| 743-761 | 621 | UAAGUGACUACCACUUAUUNN | 622 | AAUAAGUGGUAGUCACUUANN |
| 792-810 | 623 | UGUUCAGAAGUUGUUAGUGNN | 624 | CACUAACAACUUCUGAACANN |
| 799-817 | 625 | AAGUUGUUAGUGAUUUGCUNN | 626 | AGCAAAUCACUAACAACUUNN |
| 828-846 | 627 | AUAAGAUUUUUAGGUGUCUNN | 628 | AGACACCUAAAAAUCUUAUNN |
| 869-887 | 629 | AAUGACGUAUUGUGAAAUUNN | 630 | AAUUUCACAAUACGUCAUUNN |
| 920-938 | 631 | GCAUGAAACUAUGCACCUANN | 632 | UAGGUGCAUAGUUUCAUGCNN |
| 929-947 | 633 | UAUGCACCUAUAAAUACUANN | 634 | UAGUAUUUAUAGGUGCAUANN |
| 960-978 | 635 | UACCAUUUUGCGAUGUGUUNN | 636 | AACACAUCGCAAAAUGGUANN |
| 1049-1067 | 637 | AUUUUUAUCCCAUCUCACUNN | 638 | AGUGAGAUGGGAUAAAAAUNN |
| 1053-1071 | 639 | UUAUCCCAUCUCACUUUAANN | 640 | UUAAAGUGAGAUGGGAUAANN |
| 1194-1212 | 641 | UAACCCUACACUCGGAAUUNN | 642 | AAUUCCGAGUGUAGGGUUANN |
| 150-168 | 643 | AGGGUGUUCUCUAUGUAGGNN | 644 | CCUACAUAGAGAACACCCUNN |
| 158-176 | 645 | CUCUAUGUAGGCUCCAAAANN | 646 | UUUUGGAGCCUACAUAGAGNN |
| 109-127 | 647 | AACCAAACAGGGUGUGGCANN | 648 | UGCCACACCCUGUUUGGUUNN |
| 149-167 | 649 | GAGGGUGUUCUCUAUGUAGNN | 650 | CUACAUAGAGAACACCCUCNN |
| 156-174 | 651 | UUCUCUAUGUAGGCUCCAANN | 652 | UUGGAGCCUACAUAGAGAANN |
| 233-251 | 653 | GUGACAAAUGUUGGAGGAGNN | 654 | CUCCUCCAACAUUUGUCACNN |
| 329-347 | 655 | GUCAAAAAGGACCAGUUGGNN | 656 | CCAACUGGUCCUUUUUGACNN |
| 445-463 | 657 | UCAAGACUACGAACCUGAANN | 658 | UUCAGGUUCGUAGUCUUGANN |
| 633-651 | 659 | CAGCAUUUCGGUGCUUCCCNN | 660 | GGGAAGCACCGAAAUGCUGNN |
| 790-808 | 661 | GUUGUUCAGAAGUUGUUAGNN | 662 | CUAACAACUUCUGAACAACNN |
| 798-816 | 663 | GAAGUUGUUAGUGAUUUGCNN | 664 | GCAAAUCACUAACAACUUCNN |
| 800-818 | 665 | AGUUGUUAGUGAUUUGCUANN | 666 | UAGCAAAUCACUAACAACUNN |
| 805-823 | 667 | UUAGUGAUUUGCUAUCAUANN | 668 | UAUGAUAGCAAAUCACUAANN |
| 808-826 | 669 | GUGAUUUGCUAUCAUAUAUNN | 670 | AUAUAUGAUAGCAAAUCACNN |
| 868-886 | 671 | UAAUGACGUAUUGUGAAAUNN | 672 | AUUUCACAAUACGUCAUUANN |
| 928-946 | 673 | CUAUGCACCUAUAAAUACUNN | 674 | AGUAUUUAUAGGUGCAUAGNN |
| 40-58 | 675 | AUUAGCCAUGGAUGUAUUCNN | 676 | GAAUACAUCCAUGGCUAAUNN |
| 41-59 | 677 | UUAGCCAUGGAUGUAUUCANN | 678 | UGAAUACAUCCAUGGCUAANN |
| 61-79 | 679 | GAAAGGACUUUCAAAGGCCNN | 680 | GGCCUUUGAAAGUCCUUUCNN |
| 144-162 | 681 | CAAAAGAGGGUGUUCUCUANN | 682 | UAGAGAACACCCUCUUUUGNN |
| 148-166 | 683 | AGAGGGUGUUCUCUAUGUANN | 684 | UACAUAGAGAACACCCUCUNN |
| 152-170 | 685 | GGUGUUCUCUAUGUAGGCUNN | 686 | AGCCUACAUAGAGAACACCNN |
| 153-171 | 687 | GUGUUCUCUAUGUAGGCUCNN | 688 | GAGCCUACAUAGAGAACACNN |
| 154-172 | 689 | UGUUCUCUAUGUAGGCUCCNN | 690 | GGAGCCUACAUAGAGAACANN |
| 248-266 | 691 | GGAGCAGUGGUGACGGGUGNN | 692 | CACCCGUCACCACUGCUCCNN |
| 252-270 | 693 | CAGUGGUGACGGGUGUGACNN | 694 | GUCACACCCGUCACCACUGNN |
| 443-461 | 695 | UAUCAAGACUACGAACCUGNN | 696 | CAGGUUCGUAGUCUUGAUANN |
| 482-500 | 697 | CUCCCAGUUUCUUGAGAUCNN | 698 | GAUCUCAAGAAACUGGGAGNN |
| 505-523 | 699 | GACAGAUGUUCCAUCCUGUNN | 700 | ACAGGAUGGAACAUCUGUCNN |
| 540-558 | 701 | AAUGUGCCCAGUCAUGACANN | 702 | UGUCAUGACUGGGCACAUUNN |
| 544-562 | 703 | UGCCCAGUCAUGACAUUUCNN | 704 | GAAAUGUCAUGACUGGGCANN |
| 548-566 | 705 | CAGUCAUGACAUUUCUCAANN | 706 | UUGAGAAAUGUCAUGACUGNN |
| 566-584 | 707 | AAGUUUUUACAGUGUAUCUNN | 708 | AGAUACACUGUAAAAACUUNN |
| 575-593 | 709 | CAGUGUAUCUCGAAGUCUUNN | 710 | AAGACUUCGAGAUACACUGNN |
| 579-597 | 711 | GUAUCUCGAAGUCUUCCAUNN | 712 | AUGGAAGACUUCGAGAUACNN |
| 598-616 | 713 | CAGCAGUGAUUGAAGUAUCNN | 714 | GAUACUUCAAUCACUGCUGNN |
| 628-646 | 715 | CCACUCAGCAUUUCGGUGCNN | 716 | GCACCGAAAUGCUGAGUGGNN |
| 632-650 | 717 | UCAGCAUUUCGGUGCUUCCNN | 718 | GGAAGCACCGAAAUGCUGANN |
| 660-678 | 719 | AAGUGAAUACAUGGUAGCANN | 720 | UGCUACCAUGUAUUCACUUNN |
| 664-682 | 721 | GAAUACAUGGUAGCAGGGUNN | 722 | ACCCUGCUACCAUGUAUUCNN |
| 671-689 | 723 | UGGUAGCAGGGUCUUUGUGNN | 724 | CACAAAGACCCUGCUACCANN |
| 699-717 | 725 | UUUUGUGGCUUCAAUCUACNN | 726 | GUAGAUUGAAGCCACAAAANN |
| 700-718 | 727 | UUUGUGGCUUCAAUCUACGNN | 728 | CGUAGAUUGAAGCCACAAANN |
| 730-748 | 729 | AAUUAAAAACACCUAAGUGNN | 730 | CACUUAGGUGUUUUUAAUUNN |
| 731-749 | 731 | AUUAAAAACACCUAAGUGANN | 732 | UCACUUAGGUGUUUUUAAUNN |
| 736-754 | 733 | AAACACCUAAGUGACUACCNN | 734 | GGUAGUCACUUAGGUGUUUNN |
| 748-766 | 735 | GACUACCACUUAUUUCUAANN | 736 | UUAGAAAUAAGUGGUAGUCNN |
| 757-775 | 737 | UUAUUUCUAAAUCCUCACUNN | 738 | AGUGAGGAUUUAGAAAUAANN |
| 796-814 | 739 | CAGAAGUUGUUAGUGAUUUNN | 740 | AAAUCACUAACAACUUCUGNN |
| 826-844 | 741 | UUAUAAGAUUUUUAGGUGUNN | 742 | ACACCUAAAAAUCUUAUAANN |
| 827-845 | 743 | UAUAAGAUUUUUAGGUGUCNN | 744 | GACACCUAAAAAUCUUAUANN |
| 862-880 | 745 | UAAGAAUAAUGACGUAUUGNN | 746 | CAAUACGUCAUUAUUCUUANN |
| 926-944 | 747 | AACUAUGCACCUAUAAAUANN | 748 | UAUUUAUAGGUGCAUAGUUNN |
| 954-972 | 749 | AAAUUUUACCAUUUUGCGANN | 750 | UCGCAAAAUGGUAAAAUUUNN |
| 955-973 | 751 | AAUUUUACCAUUUUGCGAUNN | 752 | AUCGCAAAAUGGUAAAAUUNN |
| 957-975 | 753 | UUUUACCAUUUUGCGAUGUNN | 754 | ACAUCGCAAAAUGGUAAAANN |
| 967-985 | 755 | UUGCGAUGUGUUUUAUUCANN | 756 | UGAAUAAAACACAUCGCAANN |
| 968-986 | 757 | UGCGAUGUGUUUUAUUCACNN | 758 | GUGAAUAAAACACAUCGCANN |
| 1024-1042 | 759 | CGUUAUCUCAUUGCAAAAANN | 760 | UUUUUGCAAUGAGAUAACGNN |
| 1054-1072 | 761 | UAUCCCAUCUCACUUUAAUNN | 762 | AUUAAAGUGAGAUGGGAUANN |
| 1055-1073 | 763 | AUCCCAUCUCACUUUAAUANN | 764 | UAUUAAAGUGAGAUGGGAUNN |
| 1059-1077 | 765 | CAUCUCACUUUAAUAAUAANN | 766 | UUAUUAUUAAAGUGAGAUGNN |
| 1078-1096 | 767 | AAAUCAUGCUUAUAAGCAANN | 768 | UUGCUUAUAAGCAUGAUUUNN |
| 1083-1101 | 769 | AUGCUUAUAAGCAACAUGANN | 770 | UCAUGUUGCUUAUAAGCAUNN |
| 1106-1124 | 771 | AGAACUGACACAAAGGACANN | 772 | UGUCCUUUGUGUCAGUUCUNN |
| 1130-1148 | 773 | AUAAAGUUAUUAAUAGCCANN | 774 | UGGCUAUUAAUAACUUUAUNN |
| 1132-1150 | 775 | AAAGUUAUUAAUAGCCAUUNN | 776 | AAUGGCUAUUAAUAACUUUNN |
| 49-67 | 777 | GGAUGUAUUCAUGAAAGGANN | 778 | UCCUUUCAUGAAUACAUCCNN |
| 155-173 | 779 | GUUCUCUAUGUAGGCUCCANN | 780 | UGGAGCCUACAUAGAGAACNN |
| 512-530 | 781 | GUUCCAUCCUGUACAAGUGNN | 782 | CACUUGUACAGGAUGGAACNN |
| 763-781 | 783 | CUAAAUCCUCACUAUUUUUNN | 784 | AAAAAUAGUGAGGAUUUAGNN |
| 160-178 | 785 | CUAUGUAGGCUCCAAAACCNN | 786 | GGUUUUGGAGCCUACAUAGNN |
| 190-208 | 787 | GGUGCAUGGUGUGGCAACANN | 788 | UGUUGCCACACCAUGCACCNN |
| 494-512 | 789 | UGAGAUCUGCUGACAGAUGNN | 790 | CAUCUGUCAGCAGAUCUCANN |
| 627-645 | 791 | CCCACUCAGCAUUUCGGUGNN | 792 | CACCGAAAUGCUGAGUGGGNN |
| 669-687 | 793 | CAUGGUAGCAGGGUCUUUGNN | 794 | CAAAGACCCUGCUACCAUGNN |
| 1009-1027 | 795 | AGAAUUAAAAUAAAACGUUNN | 796 | AACGUUUUAUUUUAAUUCUNN |
| 581-599 | 797 | AUCUCGAAGUCUUCCAUCANN | 798 | UGAUGGAAGACUUCGAGAUNN |
| 916-934 | 799 | GUGAGCAUGAAACUAUGCANN | 800 | UGCAUAGUUUCAUGCUCACNN |

The details of one or more embodiments featured in the invention are set forth in the accompanying drawings and the description below. Other features, objects, and advantages of the invention will be apparent from this description, and from the claims. This application incorporates all cited references, patents, and patent applications by references in their entirety for all purposes.

### BRIEF DESCRIPTION OF DRAWINGS

FIG. 1A is the sequence of the full length mRNA of human SNCA (transcript variant NACP140; GenBank Access. No. NM_{—}000345; SEQ ID NO:1). The start and stop codons of the open reading frame are denoted in bold and italics. Sequences targeted by the siRNAs SNCA1, 3, 4, 5, 6, and 9 are underlined. Sequences of the siRNAs SNCA2, 7 and 8 are shaded in gray. SNCA1 targets nucleotides 197-217; SNCA2 targets nucleotides 205-225; SNCA3 targets nucleotides 308-330; SNCA4 targets nucleotides 231-251; SNCA5 targets nucleotides 356-376; SNCA6 targets nucleotides 261-279; SNCA7 targets nucleotides 403-421; SNCA8 targets nucleotides 451-469; SNCA9 targets nucleotides 1311-1329. Brackets flank the two alternative internal exons (exons 3 and 5). FIG. 1B is the sequence of the full length protein of human SNCA (transcript variant NACP140; GenBank Access. No. NM_{—}000345; SEQ ID NO:2).

FIG. 2A is a Western blot of EGFP or EGFP/NACP fusion proteins expressed in BE(2)-M17 human neuroblastoma cells. FIG. 2B is a bar graph showing the results of densitometric analyses of combined data from four immunoblots as shown in FIG. 2A. Data is reported as a fold value of the no-siRNA control according to EGFP:tubulin ratio. *p<0.001 Welch's modified t-test for two groups with unequal variance.

FIG. 3A is a Western blot detecting EGFP/NACP fusion proteins expressed in BE(2)-M17 human neuroblastoma cells. FIG. 3B is a graph showing the results of densitometric analyses of 0 to 5 nM samples from three independent assays as described in FIG. 3A. EGFP immunoreactivity (IR) was equalized against the tubulin IR and is expressed as a fold value relative to that of the 0 nM siRNA sample. Error bar=SEM.

FIG. 4A is a Western blot detecting EGFP/NACP fusion proteins expressed in BE(2)-M17 human neuroblastoma cells. FIG. 4B is a graph showing the results of densitometric analyses of the EGFP IR equalized against tubulin IR as described in FIG. 4A.

FIG. 5A is a Western blot detecting endogenous alpha-synuclein protein expressed in BE(2)-M17 human neuroblastoma cells. FIG. 5B is a graph showing the results of densitometric analyses of alpha-synuclein IR equalized against tubulin IR as described in FIG. 5A. *p<0.05; **p<0.01 in Student's T-test. Error bars=SEM. FIG. 5C is a graph depicting relative alpha-synuclein levels assayed by Western blot analysis.

FIG. 6A is a graph showing qRT-PCR of RNA preparations from cells treated with 50 nM siRNAs for 24 h. Each sample was assayed in quadruplicate, and expressed as a fold change from the untranslated control. *p<0.05 Welch's modified t-test for two groups with unequal variance. Error bars=SEM. FIG. 6B is a graph showing the results of a time course assay of SNCA transcription by qRT-PCR of cells transfected with 50 nM siRNA, expressed as a proportion of the untransfected control. Error bars=SEM.

FIG. 7A is a Western blot detecting human and mouse alpha-synuclein/EGFP conjugate expression. FIG. 7B is a graph depicting the activity of the siRNAs on human and mouse alpha-synuclein/EGFP conjugate expression as described in FIG. 7A. Expression was equalized using tubulin immunoreactivity and measured as a proportion of the expression of the plasmid-only EGFP immunoreactivity (control).

FIG. 8 is a qRT-PCR analysis of RNA prepared from cells treated for 24 h with 50 nM Mayo2 or Mayo9. Error bars=SEM.

FIG. 9A is a graph depicting the activity of stabilized siRNAs in cell culture assays as determined by Western blot. Immunoblots were analyzed by densitometry following 24 h contransfection with the EGFP-NACP reporter construct and defined doses of siRNA. FIG. 9B is the mean results of three qRT-PCR assays shows that endogenous SNCA mRNA is reduced significantly by stabilized siRNAs (*p<0.05; **p<0.01). Error bars=SEM.

FIG. 10 is a densitometric analysis of three independent Western blots of total protein extracts from cells transfected with plasmids conferring expression of EGFP (V; vector) or EGFP-NACP alone (control), or with 50 nM siRNAs. Error bars=SEM. *p<0.01.

FIG. 11 is a graph showing silencing of endogenous alpha-synuclein by intraparenchymal infusion of siRNA.

FIG. 12 is qRT-PCR analysis of SNCA expression following in vivo RNAi. qRT-PCR was used to determine expression of SNCA following RNAi in treated right side compared to the untreated contralateral side (R:L ratio). Open circles indicate mice in which the cannula was disconnected during treatment or did not function.

FIG. 13A is a table showing the results of in situ analysis of SNCA expression following SNCA siRNA in vivo treatment. P-values were derived from Wilcoxon rank sum test and the sample median (25th percentile-75th percentile) is given. FIG. 13B is a typical SNCA in situ from an animal treated with PBS on the right side compared to the uninjected left sides as described in FIG. 13A. FIG. 13C is a typical SNCA in situ from an animal treated with SNCA siRNA on the right side compared to the uninjected left sides as described in FIG. 13A.

FIG. 14A is a graph depicting SNCA and SNCB expression following extended timecourse of in vivo SNCA siRNA treatment. FIG. 14B is a graph depicting SNCA and SNCB expression following extended timecourse of in vivo SNCA siRNA treatment.

### DETAILED DESCRIPTION

Double-stranded (dsRNA) directs the sequence-specific silencing of mRNA through a process known as RNA interference (RNAi). The process occurs in a wide variety of organisms, including mammals and other vertebrates.

It has been demonstrated that 21-23 nt fragments of dsRNA are sequence-specific mediators of RNA silencing, e.g., by causing RNA degradation. While not wishing to be bound by theory, it may be that a molecular signal, which may be merely the specific length of the fragments, present in these 21-23 nt fragments, recruits cellular factors that mediate RNAi. Described herein are methods for preparing and administering these 21-23 nt fragments, and other dsRNAs, and their use for specifically inactivating gene function, and the function of the SNCA gene in particular. The use of dsRNAs (or recombinantly produced or chemically synthesized oligonucleotides of the same or similar nature) enables the targeting of specific mRNAs for silencing in mammalian cells. In addition, longer dsRNA agent fragments can also be used, e.g., as described below.

Although, in mammalian cells, long dsRNAs can induce the interferon response which is frequently deleterious, short dsRNAs (sRNAs) do not trigger the interferon response, at least not to an extent that is deleterious to the cell and host. In particular, the length of the dsRNA strands in an sRNA agent can be less than 31, 30, 28, 25, or 23 nt, e.g., sufficiently short to avoid inducing a deleterious interferon response. Thus, the administration of a composition of sRNA agent (e.g., formulated as described herein) to a mammalian cell can be used to silence expression of a target gene while circumventing the interferon response. Further, use of a discrete species of dsRNA can be used to selectively target one allele of a target gene, e.g., in a subject heterozygous for the allele.

Moreover, in one embodiment, a mammalian cell is treated with a dsRNA that disrupts a component of the interferon response, e.g., dsRNA-activated protein kinase PKR. Such a cell can be treated with a second dsRNA that includes a sequence complementary to a target RNA and that has a length that might otherwise trigger the interferon response.

As used herein, a “subject” refers to a mammalian organism undergoing treatment for a disorder mediated by SNCA expression. The subject can be a mammal such as a cow, horse, mouse, rat, dog, pig, goat, or a primate. In one embodiment, the subject is a human.

As used herein, disorders associated with SNCA expression refer to any biological or pathological state that (1) is mediated in part by the presence of SNCA protein and (2) whose outcome can be affected by reducing the level of SNCA protein present. Specific disorders associated with SNCA expression are noted below.

Because dsRNA mediated silencing can persist for several days after administering the dsRNA composition, in many instances, it is possible to administer the composition with a frequency of less than once per day, or, for some instances, only once for the entire therapeutic regimen.

Alpha-synuclein. Alpha-synuclein protein is primarily found in the cytoplasm, but has also been localized to the nucleus. In dopaminergic neurons, alpha-synuclein is membrane bound. The protein is a soluble monomer normally localized at the presynaptic region of axons. The protein can form filamentous aggregates that are the major component of intracellular inclusions in neurodegenerative synucleinopathies.

Alpha-synuclein protein is associated with a number of diseases characterized by synucleinopathies. Three point mutations (A53T, A30P and E46K), and SNCA duplication and triplication events are linked to autosomal dominant Parkinson's disease (familial PD, also called FPD). The A53T and A30P mutations cause configuration changes in the SNCA protein that promote in vitro protofibril formation. The triplication event results in a two-fold overexpression of SNCA protein. Alpha-synuclein is a major fibrillar component of Lewy bodies, the cytoplasmic inclusions that are characteristic of FPD and idiopathic PD, and the substantia nigra of a Parkinson's disease brain is characterized by fibrillar alpha-synuclein. In Alzheimer's patients, SNCA peptides are a major component of amyloid plaques in the brains of patients with Alzheimer's disease.

Aggregation of alpha-synuclein in the cytoplasm of cells can be caused by a number of mechanisms, including overexpression of the protein, inhibition of protein degradation, or a mutation that affects the structure of the protein, resulting in an increased tendency of the protein to self-associate.

An SNCA gene product can be a target for treatment methods of neurodegenerative diseases such as PD. The treatment methods can include targeting of an SNCA nucleic acid with a dsRNA. Alternatively, or additionally, an antisense RNA can be used to inhibit gene expression, or an antibody or small molecule can be used to target an SNCA nucleic acid. In general, an antisense RNA, anti-SNCA antibody, or small molecule can be used in place of a dsRNA, e.g., by any of the methods or compositions described herein. A combination of therapies to downregulate SNCA expression and activity can also be used.

Sequencing of the SNCA gene has revealed common variants including a dinucleotide repeat sequence (REP1) within the promoter. REP1 varies in length across populations, and certain allelic variants are associated with an increased risk for PD (Krüger et al., Ann Neurol. 45:611-7, 1999). The SNCA gene REP1 locus is necessary for normal gene expression (Touchman et al., Genome Res. 11:78-86, 2001). SNCA gene expression levels among the different REP1 alleles varied significantly over a 3-fold range, suggesting that the association of specific genotypes with an increased risk for PD may be a consequence of SNCA gene over-expression (Chiba-Falek and Nussbaum, Hum Mol. Genet. 10:3101-9, 2001). Functional analysis of intra-allelic variation at the SNCA gene REP1 locus implied that overall length of the allele plays the main role in transcriptional regulation; sequence heterogeneity is unlikely to confound genetic association studies based on alleles defined by length (Chiba-Falek et al., Hum Genet. 113:426-31, 2003). The recent discovery of SNCA gene triplication as a rare cause of PD is consistent with the observation that polymorphism within the gene promoter confers susceptibility via the same mechanism of gene over-expression (Singleton et al., Science 302:841, 2003).

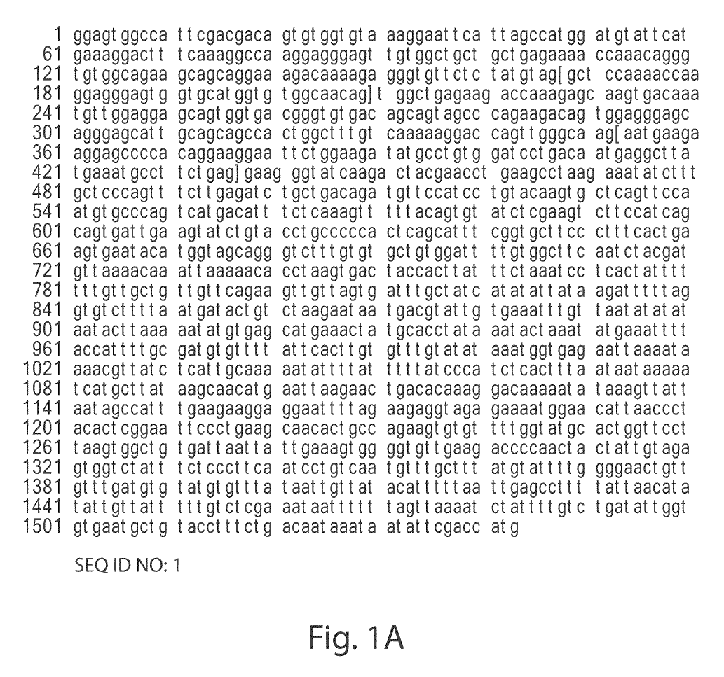

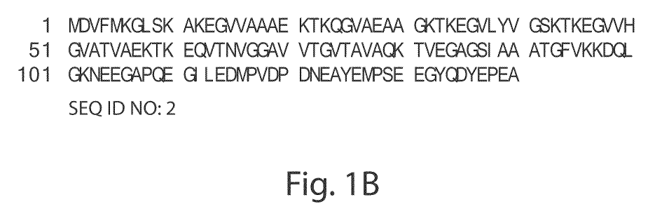

Three splice variants of SNCA have been identified (see FIG. 1A). The full-length 140 amino acid protein is the most abundant form. A 128 amino acid form lacks exon 3, and a 112 amino acid form lacks exon 5. An iRNA featured in the invention can target any isoform of SNCA. An iRNA can target a common exon (e.g., exon 2, 4, 6, or 7) to effectively target all known isoforms. A dsRNA can target a splice junction or an alternatively spliced exon to target specific isoforms. For example, to target the 112 amino acid isoform, a dsRNA can target an mRNA sequence that overlaps the exon 4/exon 6 splice junction. To target the 128 amino acid protein isoform, a dsRNA can target an mRNA sequence that overlaps the exon 2/exon 4 junction.

Treatment of Parkinson's Disease. Any patient having PD (or any other alpha-synuclein related disorder), is a candidate for treatment with a method or composition described herein. Typically, the patient is not terminally ill (e.g., the patient has life expectancy of two years or more), and has not reached end-stage Parkinson's disease (i.e., Hoehn and Yahr stage 5).

Presymptomatic subjects can also be candidates for treatment with an anti-SNCA agent, e.g., an anti-SNCA dsRNA described herein, e.g., in Tables 2, 3, or 4. In one embodiment, a presymptomatic candidate is identified by either or both of risk-factor profiling and functional neuroimaging (e.g., by fluorodopa and positron emission tomography). For example, the candidate can be identified by risk-factor profiling followed by functional neuroimaging.

Individuals having any genotype are candidates for treatment. In some embodiments the patient will carry a particular genetic mutation that places the patient at increased risk for developing PD. For example, an individual carrying an SNCA gene multiplication, e.g., an SNCA gene duplication or triplication is at increased risk for developing PD and is a candidate for treatment with the dsRNA. In addition, a gain-of-function mutation in SNCA can increase an individual's risk for developing PD. An individual carrying an SNCA REP1 genotype (e.g., a REP1 “+1 allele” heterozygous or homozygous genotype) can be a candidate for such treatment. An individual homozygous for the REP1+1 allele overexpresses SNCA. An individual carrying a mutation in the UCHL-1, parkin, or SNCA gene is at increased risk for PD and can be a candidate for treatment with an anti-SNCA dsRNA. Particularly, a mutation in the UCHL-1 or parkin gene will cause a decrease in gene or protein activity. An individual carrying a Tau genotype (e.g., a mutation in the Tau gene) or a Tau haplotype, such as the H1 haplotype is also at risk for developing PD. Other genetic risk factors include mutations in the MAPT, DJ1, PINK1, and NURR1 genes, and polymorphism in several genes including the SNCA, parkin, MAPT, and NAT2 genes.

Non-genetic (e.g., environmental) risk factors for PD include age (e.g., over age 30, 35, 40, 45, or 50 years), gender (men are generally have a higher risk than women), pesticide exposure, heavy metal exposure, and head trauma. In general, exogenous and endogenous factors that disrupt the ubiquitin proteasomal pathway or more specifically inhibit the proteasome, or which disrupt mitochondrial function, or which yield oxidative stress, or which promote the aggregation and fibrillization of alpha-synuclein, can increase the risk of an individual for developing PD, and can contribute to the pathogenesis of PD.

In one embodiment, a dsRNA can be used to target wildtype SNCA in subjects with PD.

Treatment of Other Neurodegenerative Disorders. Any disease characterized by a synucleinopathy can be treated with an inhibitory agent described herein (e.g., an agent that targets SNCA), including Lewy body dementia, Multiple System Atrophy, and Alzheimer's Disease. Individuals having any genotype are candidates for treatment. In some embodiments, the patient will carry a particular genetic mutation that places them at increased risk for developing a synucleinopathy.

In one embodiment, a dsRNA, e.g., a dsRNA described in herein, e.g., in Tables 2, 3, or 4, can be used to target wildtype SNCA in subjects with a neurodegenerative disorder.

An individual can develop a synucleinopathy as a result of certain environmental factors. For example, oxidative stress, certain pesticides (e.g., 24D and agent orange), bacterial infection, and head trauma have been linked to an increase in the risk of developing PD, and can be determining factors for determining the risk of an individual for synucleinopathies. These factors (and others disclosed herein) can be considered when evaluating the risk profile of a candidate subject for anti-SNCA therapy.

### I. DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A” and “U” each generally stand for a nucleotide that contains guanine, cytosine, adenine, and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide including a nucleotide bearing such replacement moiety. For example, without limitation, a nucleotide including inosine as its base may base pair with nucleotides containing adenine, cytosine, or uracil. Hence, nucleotides containing uracil, guanine, or adenine may be replaced in the nucleotide sequences featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences including such replacement moieties are embodiments featured in the invention.

By “SNCA” as used herein is meant a SNCA mRNA, protein, peptide, or polypeptide. The term “SNCA” is also known in the art as alpha-synuclein.

As used herein, “target sequence” refers to a contiguous portion of the nucleotide sequence of an mRNA molecule formed during the transcription of the SNCA gene, including mRNA that is a product of RNA processing of a primary transcription product.

As used herein, the term “strand including a sequence” refers to an oligonucleotide including a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide including the first nucleotide sequence to hybridize and form a duplex structure under certain conditions with an oligonucleotide or polynucleotide including the second nucleotide sequence, as will be understood by the skilled person. For substantial complementarity, such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide including the first nucleotide sequence to the oligonucleotide or polynucleotide including the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA including one oligonucleotide 21 nucleotides in length and another oligonucleotide 23 nucleotides in length, wherein the longer oligonucleotide includes a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of” a messenger RNA (mRNA) refers to a polynucleotide which is substantially complementary to a contiguous portion of the mRNA of interest (e.g., encoding SNCA). For example, a polynucleotide is complementary to at least a part of a SNCA mRNA if the sequence is substantially complementary to a non-interrupted portion of an mRNA encoding SNCA.

The term “double-stranded RNA” or “dsRNA”, as used herein, refers to a ribonucleic acid molecule, or complex of ribonucleic acid molecules, having a duplex structure including two anti-parallel and substantially complementary, as defined above, nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker.” The RNA strands may have the same or a different number of nucleotides. The maximum number of base pairs is the number of nucleotides in the shortest strand of the dsRNA. In addition to the duplex structure, a dsRNA may comprise one or more nucleotide overhangs. A dsRNA as used herein is also referred to as a “small inhibitory RNA” or “siRNA.”

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to the corresponding segment of a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

The term “identity” is the relationship between two or more polynucleotide sequences, as determined by comparing the sequences. Identity also means the degree of sequence relatedness between polynucleotide sequences, as determined by the match between strings of such sequences. While there exist a number of methods to measure identity between two polynucleotide sequences, the term is well known to skilled artisans (see, e.g., Sequence Analysis in Molecular Biology, von Heinje, G., Academic Press (1987); and Sequence Analysis Primer, Gribskov, M. and Devereux, J., eds., M. Stockton Press, New York (1991)). “Substantially identical,” as used herein, means there is a very high degree of homology (e.g., 100% sequence identity) between the sense strand of the dsRNA and the corresponding part of the target gene. However, dsRNA having greater than 90%, or 95% sequence identity may be used in the present invention, and thus sequence variations that might be expected due to genetic mutation, strain polymorphism, or evolutionary divergence can be tolerated. Although 100% identity is typical, the dsRNA may contain single or multiple base-pair random mismatches between the RNA and the target gene.

As used herein, the term “SNALP” refers to a stable nucleic acid-lipid particle. A SNALP represents a vesicle of lipids coating a reduced aqueous interior comprising a nucleic acid such as an iRNA agent or a plasmid from which an iRNA agent is transcribed. SNALPs are described, e.g., in U.S. Patent Application Publication Nos. 20060240093, 20070135372, and U.S. Ser. No. 61/045,228 filed Apr. 15, 2008. These applications are hereby incorporated by reference.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell,” wherein the cell is part of a living organism. In such instance, introduction into the cell will include the delivery to the organism. For example, for in vivo delivery, dsRNA can be injected into a tissue site or administered systemically. In vitro introduction into a cell includes methods known in the art such as electroporation and lipofection.

The terms “silence” and “inhibit the expression of,” insofar as they refer to the SNCA gene, herein refer to the at least partial suppression of the expression of the SNCA gene, as manifested by a reduction of the amount of mRNA transcribed from the SNCA gene which may be isolated from a first cell or group of cells in which the SNCA gene is transcribed and which has or have been treated such that the expression of the SNCA gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to SNCA gene transcription, e.g. the amount of protein encoded by the SNCA gene which is present on the cell surface, or the number of cells displaying a certain phenotype, e.g apoptosis. In principle, SNCA gene silencing may be determined in any cell expressing the target, either constitutively or by genomic engineering, and by any appropriate assay. However, when a reference is needed in order to determine whether a given siRNA inhibits the expression of the SNCA gene by a certain degree and therefore is encompassed by the instant invention, the assays provided in the Examples below shall serve as such reference.

For example, in certain instances, expression of the SNCA gene is suppressed by at least about 20%, 25%, 35%, or 40% by administration of the double-stranded oligonucleotide featured in the invention. In one embodiment, the SNCA gene is suppressed by at least about 50%, 60%, or 70% by administration of the double-stranded oligonucleotide featured in the invention. In another embodiment, the SNCA gene is suppressed by at least about 75%, 80%, 90% or 95% by administration of the double-stranded oligonucleotide featured in the invention.

The terms “treat,” “treatment,” and the like, refer to relief from or alleviation of an neurodegenerative disease, such as a synucleinopathy. In the context of the present invention insofar as it relates to any of the other conditions recited herein below (e.g., a SNCA-mediated condition other than an neurodegenerative disease), the terms “treat,” “treatment,” and the like mean to relieve or alleviate at least one symptom associated with such condition, or to slow or reverse the progression of such condition.

As used herein, the term “SNCA-mediated condition or disease” and related terms and phrases refer to a condition or disorder characterized by inappropriate, e.g., greater than normal, SNCA activity. Inappropriate SNCA functional activity might arise as the result of SNCA expression in cells which normally do not express SNCA, or increased SNCA expression (leading to, e.g., neurodegenerative disease). A SNCA-mediated condition or disease may be completely or partially mediated by inappropriate SNCA functional activity. However, a SNCA-mediated condition or disease is one in which modulation of SNCA results in some effect on the underlying condition or disorder (e.g., a SNCA inhibitor results in some improvement in patient well-being in at least some patients).

As used herein, the phrases “therapeutically effective amount” and “prophylactically effective amount” refer to an amount that provides a therapeutic benefit in the treatment, prevention, or management of a neurodegenerative disorder, such as synucleinopathy, e.g., Parkinson's Disease. The specific amount that is therapeutically effective can be readily determined by ordinary medical practitioner, and may vary depending on factors known in the art, such as, e.g. the type of neurodegenerative disease, the patient's history and age, the stage of the disease, and the administration of other agents.

As used herein, a “pharmaceutical composition” includes a pharmacologically effective amount of a dsRNA and a pharmaceutically acceptable carrier. As used herein, “pharmacologically effective amount,” “therapeutically effective amount” or simply “effective amount” refers to that amount of a RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a vector has been introduced from which a dsRNA molecule may be expressed.

### II. DOUBLE-STRANDED RIBONUCLEIC ACID (dsRNA)

In one embodiment, the invention provides double-stranded ribonucleic acid (dsRNA) molecules for inhibiting the expression of the SNCA gene in a cell or mammal. The dsRNA includes an antisense strand including a region of complementarity which is complementary to the corresponding region of an mRNA formed in the expression of the SNCA gene, and wherein the region of complementarity is less than 19, 20, 21, 22, 23, 24, 25, or 30 nucleotides in length, and is generally 15-30, 18-25, 19-24 or 21-23 nucleotides in length. In one embodiment, the region of complementarity is at least 10, 15, 16, 17, or 18 nucleotides in length. In one embodiment the dsRNA, upon contact with a cell expressing said SNCA gene, inhibits the expression of said SNCA gene, e.g., in an assay to test SNCA expression. The dsRNA includes two RNA strands that are sufficiently complementary to hybridize to form a duplex structure. The sense strand includes a region which is complementary to the antisense strand, such that the two strands hybridize and form a duplex structure when combined under suitable conditions. Generally, the duplex structure is between 15 and 30, more generally between 18 and 25, yet more generally between 18 and 20, or 19 and 24, and most generally between 21 and 23 base pairs in length. The dsRNA featured in the invention may further include one or more single-stranded nucleotide overhang(s). The dsRNA can be synthesized by standard methods known in the art as further discussed below, e.g., by use of an automated DNA synthesizer, such as are commercially available from, for example, Biosearch, Applied Biosystems, Inc. In another embodiment, the SNCA gene is the human SNCA gene. In specific embodiments, the dsRNA has a first sequence selected from the group consisting of the sense sequences of Tables 2, 3, and 4, and a second sequence selected from the group consisting of the antisense sequences of Tables 2, 3, and 4.

In further embodiments, the dsRNA includes at least one nucleotide sequence selected from the groups of sequences provided in Tables 2, 3, and 4. In other embodiments, the dsRNA includes at least two sequences selected from this group, wherein one of the at least two sequences is complementary to another of the at least two sequences, and one of the at least two sequences is substantially complementary to a sequence of an mRNA generated in the expression of the SNCA gene. Generally, the dsRNA includes two oligonucleotides, wherein one oligonucleotide is described as the sense strand in Tables 2, 3, or 4, and the second oligonucleotide is described as the antisense strand in Tables 2, 3, or 4.

The skilled person is well aware that dsRNAs including a duplex structure of between 20 and 23, but specifically 21, base pairs have been identified as particularly effective in inducing RNA interference (Elbashir et al., EMBO 2001, 20:6877-6888). However, others have found that shorter or longer dsRNAs can be effective as well. In the embodiments described above, by virtue of the nature of the oligonucleotide sequences provided in Tables 2, 3, and 4, the dsRNAs featured in the invention can include at least one strand of a length of minimally 21 nt. It can be reasonably expected that shorter dsRNAs including one of the sequences of Tables 2, 3, or 4 minus only a few nucleotides on one or both ends may be similarly effective as compared to the dsRNAs described above. Hence, dsRNAs including a partial sequence of at least 15, 16, 17, 18, 19, 20, or more contiguous nucleotides from one of the sequences of Tables 2, 3, or 4, and differing in their ability to inhibit the expression of the SNCA gene in a FACS assay as described herein below by not more than 5, 10, 15, 20, 25, or 30% inhibition from a dsRNA including the full sequence, are contemplated by the invention.

In addition, the RNAi agents provided in Tables 2, 3, and 4 identify sites in the SNCA mRNA that are susceptible to RNAi based cleavage. As such, the invention further includes RNAi agents that target within the sequence targeted by one of the agents of the present invention. As used herein a second RNAi agent is said to target within the sequence of a first RNAi agent if the second RNAi agent cleaves the message anywhere within the mRNA that is complementary to the antisense strand of the first RNAi agent. Such a second agent will generally consist of at least 15 contiguous nucleotides from one of the sequences provided in Tables 2, 3, and 4 coupled to additional nucleotide sequences taken from the region contiguous to the selected sequence in the SNCA gene.

The dsRNA featured in the invention can contain one or more mismatches to the target sequence. In one embodiment, the dsRNA featured in the invention contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity. For example, for a 23 nucleotide dsRNA strand which is complementary to a region of the SNCA gene, the dsRNA generally does not contain any mismatch within the central 13 nucleotides. The methods described within the invention can be used to determine whether a dsRNA containing a mismatch to a target sequence is effective in inhibiting the expression of the SNCA gene. Consideration of the efficacy of dsRNAs with mismatches in inhibiting expression of the SNCA gene is important, especially if the particular region of complementarity in the SNCA gene is known to have polymorphic sequence variation within the population.

In one embodiment, at least one end of the dsRNA has a single-stranded nucleotide overhang of 1 to 4, generally 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The dsRNA may also have a blunt end, generally located at the 5′-end of the antisense strand. Such dsRNAs have improved stability and inhibitory activity, thus allowing administration at low dosages, i.e., less than 5 mg/kg body weight of the recipient per day. In one embodiment, the antisense strand of the dsRNA has 1-10 nucleotides overhangs each at the 3′ end and the 5′ end over the sense strand. In one embodiment, the sense strand of the dsRNA has 1-10 nucleotides overhangs each at the 3′ end and the 5′ end over the antisense strand. In another embodiment, one or more of the nucleotides in the overhang is replaced with a nucleoside thiophosphate.

In yet another embodiment, the dsRNA is chemically modified to enhance stability. The nucleic acids featured in the invention may be synthesized and/or modified by methods well established in the art, such as those described in “Current protocols in nucleic acid chemistry”, Beaucage, S. L. et al. (Edrs.), John Wiley & Sons, Inc., New York, N.Y., USA, which is hereby incorporated herein by reference. Specific examples of dsRNA compounds useful in this invention include dsRNAs containing modified backbones or no natural internucleoside linkages. As defined in this specification, dsRNAs having modified backbones include those that retain a phosphorus atom in the backbone and those that do not have a phosphorus atom in the backbone. For the purposes of this specification, and as sometimes referenced in the art, modified dsRNAs that do not have a phosphorus atom in their internucleoside backbone can also be considered to be oligonucleosides.

Modified dsRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is herein incorporated by reference

Modified dsRNA backbones that do not include a phosphorus atom therein have backbones that are formed by short chain alkyl or cycloalkyl internucleoside linkages, mixed heteroatoms and alkyl or cycloalkyl internucleoside linkages, or ore or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other dsRNA mimetics, both the sugar and the internucleoside linkage, i.e., the backbone, of the nucleotide units are replaced with novel groups. The base units are maintained for hybridization with an appropriate nucleic acid target compound. One such oligomeric compound, a dsRNA mimetic that has been shown to have excellent hybridization properties, is referred to as a peptide nucleic acid (PNA). In PNA compounds, the sugar backbone of a dsRNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Typical embodiments featured in the invention include dsRNAs with phosphorothioate backbones and oligonucleosides with heteroatom backbones, and in particular —CH.sub.2-NH—CH.sub.2-, —CH.sub.2-N(CH.sub.3)-O—CH.sub.2-[known as a methylene (methylimino) or MMI backbone], —CH.sub.2-O—N(CH.sub.3)-CH.sub.2-, —CH.sub.2-N(CH.sub.3)-N(CH.sub.3)-CH.sub.2- and —N(CH.sub.3)-CH.sub.2-CH.sub.2-[wherein the native phosphodiester backbone is represented as —O—P—O—CH.sub.2-] of the above-referenced U.S. Pat. No. 5,489,677, and the amide backbones of the above-referenced U.S. Pat. No. 5,602,240. Also featured in the invention are dsRNAs having morpholino backbone structures of the above-referenced U.S. Pat. No. 5,034,506.

Modified dsRNAs may also contain one or more substituted sugar moieties. Typical dsRNAs include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C.sub.10 alkyl or C.sub.2 to C.sub.10 alkenyl and alkynyl. Other modifications include O[(CH.sub.2).sub.nO].sub.mCH.sub.3, O(CH.sub.2).sub.nOCH.sub.3, O(CH.sub.2).sub.nNH.sub.2, O(CH.sub.2).sub.nCH.sub.3, O(CH.sub.2).sub.nONH.sub.2, and O(CH.sub.2).sub.nON[(CH.sub.2).sub.nCH.su-b.3)].sub.2, where n and m are from 1 to about 10. Other dsRNAs include one of the following at the 2′ position: C.sub.1 to C.sub.10 lower alkyl, substituted lower alkyl, alkaryl, aralkyl, O-alkaryl or O-aralkyl, SH, SCH.sub.3, OCN, Cl, Br, CN, CF.sub.3, OCF.sub.3, SOCH.sub.3, SO.sub.2CH.sub.3, ONO.sub.2, NO.sub.2, N.sub.3, NH.sub.2, heterocycloalkyl, heterocycloalkaryl, aminoalkylamino, polyalkylamino, substituted silyl, an RNA cleaving group, a reporter group, an intercalator, a group for improving the pharmacokinetic properties of an dsRNA, or a group for improving the pharmacodynamic properties of an dsRNA, and other substituents having similar properties. Certain modifications include 2′-methoxyethoxy(2′-O—CH.sub.2CH.sub.2OCH.sub.3, also known as 2′-O-(2-methoxyethyl) or 2′-MOE) (Martin et al., Helv. Chim. Acta, 1995, 78, 486-504) i.e., an alkoxy-alkoxy group. Other modifications include 2′-dimethylaminooxyethoxy, i.e., a O(CH.sub.2).sub.20N(CH.sub.3).sub.2 group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH.sub.2-O—CH.sub.2-N(CH.sub.2).sub.2, also described in examples hereinbelow.

Other typical modifications include 2′-methoxy(2′-OCH.sub.3), 2′-aminopropoxy(2′-OCH.sub.2CH.sub.2CH.sub.2NH.sub.2) and 2′-fluoro (2′-F). Similar modifications may also be made at other positions on the dsRNA, particularly the 3′ position of the sugar on the 3′ terminal nucleotide or in 2′-5′ linked dsRNAs and the 5′ position of 5′ terminal nucleotide. DsRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

dsRNAs may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, DsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., Eds., DsRNA Research and Applications, CRC Press, Boca Raton, 1993, pp. 276-278) and are typical base substitutions, particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference, and U.S. Pat. No. 5,750,692, also herein incorporated by reference.

Another modification of the dsRNAs featured in the invention involves chemically linking to the dsRNA one or more moieties or conjugates which enhance the activity, cellular distribution or cellular uptake of the dsRNA. Such moieties include but are not limited to lipid moieties such as a cholesterol moiety (Letsinger et al., Proc. Natl. Acid. Sci. USA, 199, 86, 6553-6556), cholic acid (Manoharan et al., Biorg. Med. Chem. Let., 1994 4 1053-1060), a thioether, e.g., beryl-5-tritylthiol (Manoharan et al., Ann. N.Y. Acad. Sci., 1992, 660, 306-309; Manoharan et al., Biorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyloxycholesterol moiety (Crooke et al., J. Pharmacol. Exp. Ther., 1996, 277, 923-937).

Representative U.S. patents that teach the preparation of such dsRNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an dsRNA. The present invention also includes dsRNA compounds which are chimeric compounds. “Chimeric” dsRNA compounds or “chimeras,” in the context of this invention, are dsRNA compounds, particularly dsRNAs, which contain two or more chemically distinct regions, each made up of at least one monomer unit, i.e., a nucleotide in the case of a dsRNA compound. These dsRNAs typically contain at least one region wherein the dsRNA is modified so as to confer upon the dsRNA increased resistance to nuclease degradation, increased cellular uptake, and/or increased binding affinity for the target nucleic acid. An additional region of the dsRNA may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of dsRNA inhibition of gene expression. Consequently, comparable results can often be obtained with shorter dsRNAs when chimeric dsRNAs are used, compared to phosphorothioate deoxydsRNAs hybridizing to the same target region. Cleavage of the RNA target can be routinely detected by gel electrophoresis and, if necessary, associated nucleic acid hybridization techniques known in the art.

In certain instances, the dsRNA may be modified by a non-ligand group. A number of non-ligand molecules have been conjugated to dsRNAs in order to enhance the activity, cellular distribution or cellular uptake of the dsRNA, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such dsRNA conjugates have been listed above. Typical conjugation protocols involve the synthesis of dsRNAs bearing an aminolinker at one or more positions of the sequence. The amino group is then reacted with the molecule being conjugated using appropriate coupling or activating reagents. The conjugation reaction may be performed either with the dsRNA still bound to the solid support or following cleavage of the dsRNA in solution phase. Purification of the dsRNA conjugate by HPLC typically affords the pure conjugate. Inclusion of a cholesterol conjugate is particularly useful for targeting vaginal epithelium cells, a site of SNCA expression.

Vector Encoded RNAi Agents

The dsRNA featured in the invention can also be expressed from recombinant viral vectors intracellularly in vivo. The recombinant viral vectors featured in the invention include sequences encoding the dsRNA featured in the invention and any suitable promoter for expressing the dsRNA sequences. Suitable promoters include, for example, the U6 or H1 RNA pol III promoter sequences and the cytomegalovirus promoter. Selection of other suitable promoters is within the skill in the art. The recombinant viral vectors featured in the invention can also comprise inducible or regulatable promoters for expression of the dsRNA in a particular tissue or in a particular intracellular environment. The use of recombinant viral vectors to deliver dsRNA to cells in vivo is discussed in more detail below.

dsRNA featured in the invention can be expressed from a recombinant viral vector either as two separate, complementary RNA molecules, or as a single RNA molecule with two complementary regions.

Any viral vector capable of accepting the coding sequences for the dsRNA molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

For example, lentiviral vectors can be pseudotyped with surface proteins from vesicular stomatitis virus (VSV), rabies, Ebola, Mokola, and the like. AAV vectors can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the dsRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference.

Typical viral vectors are those derived from AV and AAV. In a one embodiment, the dsRNA featured in the invention is expressed as two separate, complementary single-stranded RNA molecules from a recombinant AAV vector including, for example, either the U6 or H1 RNA promoters, or the cytomegalovirus (CMV) promoter.

A suitable AV vector for expressing a dsRNA featured in the invention, a method for constructing the recombinant AV vector, and a method for delivering the vector into target cells, are described in Xia H et al. (2002), Nat. Biotech. 20: 1006-1010.

Suitable AAV vectors for expressing dsRNA, e.g., dsRNA targeting SNCA, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

### III. PHARMACEUTICAL COMPOSITIONS INCLUDING dsRNA

In one embodiment, the invention provides pharmaceutical compositions including a dsRNA, as described herein, and a pharmaceutically acceptable carrier. The pharmaceutical composition including the dsRNA is useful for treating a disease or disorder associated with the expression or activity of the SNCA gene, such as pathological processes mediated by SNCA expression. Such pharmaceutical compositions are formulated based on the mode of delivery. One example is compositions that are formulated for systemic administration via parenteral delivery.

The pharmaceutical compositions featured in the invention are administered in dosages sufficient to inhibit expression of the SNCA gene. The present inventors have found that, because of their improved efficiency, compositions including the dsRNA can be administered at surprisingly low dosages. Dosages of 0.6 mg or greater of dsRNA per kilogram body weight of recipient per day is sufficient to suppress expression of the SNCA gene by greater than 35%, with higher dosages capable of achieving 65% reduction in expression of the SNCA gene.

In general, a suitable dose of dsRNA will be in the range of 0.01 to 5.0 milligrams per kilogram body weight of the recipient per day, generally in the range of 1 microgram to 1 mg per kilogram body weight per day. The pharmaceutical composition may be administered once daily, or the dsRNA may be administered as two, three, or more sub-doses at appropriate intervals throughout the day or even using continuous infusion or delivery through a controlled release formulation. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art and are particularly useful for vaginal delivery of agents, such as could be used with the agents of the present invention. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

Advances in mouse genetics have generated a number of mouse models for the study of various human diseases, such as pathological processes mediated by SNCA expression. Such models are used for in vivo testing of dsRNA, as well as for determining a therapeutically effective dose.

The present invention also includes pharmaceutical compositions and formulations which include the dsRNA compounds featured in the invention. The pharmaceutical compositions of the present invention may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal), oral or parenteral. Administration may also be designed to result in preferential localization to particular tissues through local delivery, e.g. by direct intraarticular injection into joints, by rectal administration for direct delivery to the gut and intestines, by intravaginal administration for delivery to the cervix and vagina, by intravitreal administration for delivery to the eye. Parenteral administration includes intravenous, intraarterial, intraarticular, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Typical topical formulations include those in which dsRNAs targeting SNCA are in admixture with a topical delivery agent such as lipids, liposomes, fatty acids, fatty acid esters, steroids, chelating agents and surfactants. Typical lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). DsRNAs featured in the invention may be encapsulated within liposomes or may form complexes thereto, in particular to cationic liposomes. Alternatively, dsRNAs may be complexed to lipids, in particular to cationic lipids. Typical fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

In one embodiment, an SNCA dsRNA featured in the invention is fully encapsulated in the lipid formulation (e.g., to form a SPLP, pSPLP, SNALP, or other nucleic acid-lipid particle). As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), or a mixture thereof. The cationic lipid may comprise from about 20 mol % to about 50 mol % or about 40 mol % of the total lipid present in the particle.

The non-cationic lipid may be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may be from about 5 mol % to about 90 mol %, about 10 mol %, or about 58 mol % if cholesterol is included, of the total lipid present in the particle.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles may be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In one embodiment, the lipidoid ND98.4HCl (MW 1487) (Formula 1), Cholesterol (Sigma-Aldrich), and PEG-Ceramide C16 (Avanti Polar Lipids) can be used to prepare lipid-siRNA nanoparticles (i.e., LNP01 particles). Stock solutions of each in ethanol can be prepared as follows: ND98, 133 mg/mL; Cholesterol, 25 mg/mL, PEG-Ceramide C16, 100 mg/mL. The ND98, Cholesterol, and PEG-Ceramide C16 stock solutions can then be combined in a, e.g., 42:48:10 molar ratio. The combined lipid solution can be mixed with aqueous siRNA (e.g., in sodium acetate pH 5) such that the final ethanol concentration is about 35-45% and the final sodium acetate concentration is about 100-300 mM. Lipid-siRNA nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

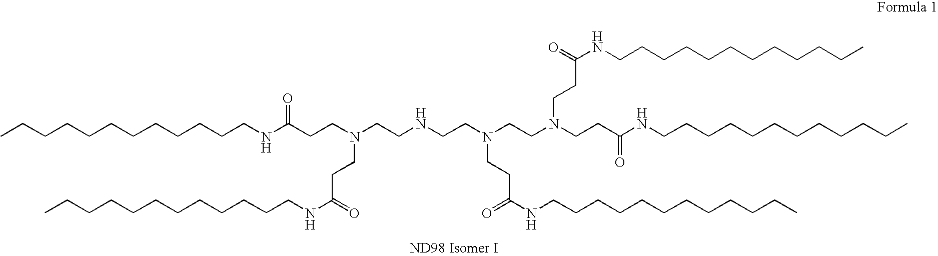

LNP01 formulations are described, e.g., in International Application Publication No. WO 2008/042973, which is hereby incorporated by reference.

Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, USA). Particles should be about 20-300 nm, such as 40-100 nm in size. The particle size distribution should be unimodal. The total siRNA concentration in the formulation, as well as the entrapped fraction, is estimated using a dye exclusion assay. A sample of the formulated siRNA can be incubated with an RNA-binding dye, such as Ribogreen (Molecular Probes) in the presence or absence of a formulation disrupting surfactant, e.g., 0.5% Triton-X100. The total siRNA in the formulation can be determined by the signal from the sample containing the surfactant, relative to a standard curve. The entrapped fraction is determined by subtracting the “free” siRNA content (as measured by the signal in the absence of surfactant) from the total siRNA content. Percent entrapped siRNA is typically >85%. For SNALP formulation, the particle size is at least 30 nm, at least 40 nm, at least 50 nm, at least 60 nm, at least 70 nm, at least 80 nm, at least 90 nm, at least 100 nm, at least 110 nm, and at least 120 nm. The suitable range is typically about at least 50 nm to about at least 110 nm, about at least 60 nm to about at least 100 nm, or about at least 80 nm to about at least 90 nm.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Typical oral formulations are those in which dsRNAs featured in the invention are administered in conjunction with one or more penetration enhancers surfactants and chelators. Typical surfactants include fatty acids and/or esters or salts thereof, bile acids and/or salts thereof. Typical bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Typical fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Combinations of penetration enhancers are also suitable, such as fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the invention may be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Typical complexing agents include chitosan, N-trimethylchitosan, poly-L-lysine, polyhistidine, polyomithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. application. Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999), each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The compositions of the present invention may be prepared and formulated as emulsions. Emulsions are typically heterogenous systems of one liquid dispersed in another in the form of droplets usually exceeding 0.1 .mu.m in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems including two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of dsRNAs and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that the microemulsion compositions and formulations of the present invention will facilitate the increased systemic absorption of dsRNAs and nucleic acids from the gastrointestinal tract, as well as improve the local cellular uptake of dsRNAs and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the dsRNAs and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems including non-ionic surfactant and cholesterol. Non-ionic liposomal formulations including Novasome.TM. I (glyceryl dilaurate/cholesterol/po-lyoxyethylene-10-stearyl ether) and Novasome.TM. II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S. T. P. Pharma. Sci., 1994, 4, 6, 466).

Liposomes also include “sterically stabilized” liposomes, a term which, as used herein, refers to liposomes including one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) includes one or more glycolipids, such as monosialoganglioside G.sub.M1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes including one or more glycolipids are known in the art. Papahadjopoulos et al. (Ann. N.Y. Acad. Sci., 1987, 507, 64) reported the ability of monosialoganglioside G.sub.M1, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes including (1) sphingomyelin and (2) the ganglioside G.sub.M1 or a galactocerebroside sulfate ester. U.S. Pat. No. 5,543,152 (Webb et al.) discloses liposomes including sphingomyelin. Liposomes including 1,2-sn-dimyristoylphosphat-idylcholine are disclosed in WO 97/13499 (Lim et al).

Many liposomes including lipids derivatized with one or more hydrophilic polymers, and methods of preparation thereof, are known in the art. Sunamoto et al. (Bull. Chem. Soc. Jpn., 1980, 53, 2778) described liposomes including a nonionic detergent, 2C.sub.1215G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes including phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes including a number of other lipid-polymer conjugates are disclosed in WO 91/05545 and U.S. Pat. No. 5,225,212 (both to Martin et al.) and in WO 94/20073 (Zalipsky et al.) Liposomes including PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes including nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an dsRNA RNA. U.S. Pat. No. 5,665,710 to Rahman et al. describes certain methods of encapsulating oligodeoxynucleotides in liposomes. WO 97/04787 to Love et al. discloses liposomes including dsRNA dsRNAs targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Penetration Enhancers

In one embodiment, the present invention employs various penetration enhancers to effect the efficient delivery of nucleic acids, particularly dsRNAs, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of dsRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C.sub.1-10 alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts featured in the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of dsRNAs through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents featured in the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of dsRNAs through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of dsRNAs at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments featured herein provide pharmaceutical compositions containing (a) one or more antisense compounds and (b) one or more other therapeutic agents which function by a non-antisense mechanism. For example, the one or more other therapeutic agents can be from the classes of cholinesterase inhibitors, muscarinic agonists, anti-oxidants or anti-inflammatories. Exemplary therapeutics include acetyl-L-carnitine, vinpocetine, huperzine A, alpha lipoic acid, vitamin E, rhodiola, biotin, galantamine (Reminyl), tacrine (Cognex), selegiline, physostigmine, revistigmin, donepezil, (Aricept), rivastigmine (Exelon), metrifonate, milameline, xanomeline, saeluzole, idebenone, ENA-713, mermic, quetiapine, neurestrol, idebenone, propentofylline, and neuromidal.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are particularly useful for the methods featured herein.

The data obtained from cell culture assays and animal studies can be used in formulation a range of dosage for use in humans. The dosage of compositions featured in the invention lies generally within a range of circulating concentrations that include the ED50 with little or no toxicity. The dosage may vary within this range depending upon the dosage form employed and the route of administration utilized. For any compound used in the method described herein, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, the dsRNAs featured in the invention can be administered in combination with other known agents effective in treatment of pathological processes mediated by SNCA expression. In any event, the administering physician can adjust the amount and timing of dsRNA administration on the basis of results observed using standard measures of efficacy known in the art or described herein.

Methods for Treating Diseases Caused by Expression of the SNCA Gene

In one embodiment, the invention provides a method for treating a subject having a pathological condition mediated by the expression of the SNCA gene, such as an neurodegenerative disease, such as a synucleinopathy, such as Parkinson's Disease. In this embodiment, the dsRNA acts as a therapeutic agent for controlling the expression of the SNCA protein. The method includes administering a pharmaceutical composition featured in the invention to the patient (e.g., human), such that expression of the SNCA gene is silenced. Because of their high specificity, the dsRNAs described herein specifically target mRNAs of the SNCA gene.

As used herein, the term “SNCA-mediated condition or disease” and related terms and phrases refer to a condition or disorder characterized by unwanted or inappropriate, e.g., abnormal SNCA activity. Inappropriate SNCA functional activity might arise as the result of SNCA expression in cells which normally do not express SNCA, increased SNCA expression and/or activity (leading to, e.g., neurogenerative disease, or increased susceptibility to disease). A SNCA-mediated condition or disease may be completely or partially mediated by inappropriate SNCA functional activity which may result by way of inappropriate activation of SNCA. Regardless, a SNCA-mediated condition or disease is one in which modulation of SNCA via RNA interference results in some effect on the underlying condition or disorder (e.g., a SNCA inhibitor results in some improvement in patient well-being in at least some patients).

The anti-SNCA compounds of the present invention may be used to treat or diagnose a neurodegenerative disease in a subject. The methods include administering to a subject an anti-SNCA compound featured herein in an amount effective to treat a neurodegenerative disease or disorder.

Pathological processes refer to a category of biological processes that produce a deleterious effect. For example, unregulated expression of SNCA is associated with neurodegenerative disease. A compound featured in the invention can typically modulate a pathological process when the compound reduces the degree or severity of the process. For instance, neurodegeneration, or a synucleinopathy, may be prevented or related pathological processes can be modulated by the administration of compounds that reduce or modulate in some way the expression or at least one activity SNCA.

The dsRNA molecules featured herein may, therefore, be used to treat neurodegenerative diseases, such as Parkinson's disease, Alzheimer's disease, multiple system atrophy, and Lewy body dementia. The dsRNA molecules featured herein are also useful for the treatment of a retinal disorder, e.g., a retinopathy.

The pharmaceutical compositions encompassed by the invention may be administered by any means known in the art including, but not limited to oral or parenteral routes, including intravenous, intramuscular, intraarticular, intraperitoneal, subcutaneous, intravitreal, transdermal, airway (aerosol), nasal, rectal, vaginal and topical (including buccal and sublingual) administration, and epidural administration. In certain embodiments, the pharmaceutical compositions are administered intravenously by infusion or injection.

Methods for Inhibiting Expression of the SNCA Gene

In yet another aspect, the invention provides a method for inhibiting the expression of the SNCA gene in a mammal. The method includes administering a composition featured herein to the mammal such that expression of the target SNCA gene is silenced. Because of their high specificity, the dsRNAs featured in the invention specifically target RNAs (primary or processed) of the target SNCA gene. Compositions and methods for inhibiting the expression of the SNCA gene using dsRNAs can be performed as described elsewhere herein.

In one embodiment, the method includes administering a composition including a dsRNA, wherein the dsRNA includes a nucleotide sequence which is complementary to at least a part of an RNA transcript of the SNCA gene of the mammal to be treated. When the organism to be treated is a mammal such as a human, the composition may be administered by any means known in the art including, but not limited to oral or parenteral routes, including intravenous, intramuscular, intraarticular, intracranial, subcutaneous, intravitreal, transdermal, airway (aerosol), nasal, rectal, vaginal and topical (including buccal and sublingual) administration. In some embodiments, the compositions are administered by intraveneous infusion or injection.

dsRNA Expression Vectors

In another aspect, SNCA specific dsRNA molecules that modulate SNCA gene expression activity are expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In one embodiment, a dsRNA is expressed as an inverted repeat joined by a linker polynucleotide sequence such that the dsRNA has a stem and loop structure.

The recombinant dsRNA expression vectors are generally DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Eglitis, et al., Science (1985) 230:1395-1398; Danos and Mulligan, Proc. NatI. Acad. Sci. USA (1998) 85:6460-6464; Wilson et al., 1988, Proc. NatI. Acad. Sci. USA 85:3014-3018; Armentano et al., 1990, Proc. NatI. Acad. Sci. USA 87:61416145; Huber et al., 1991, Proc. NatI. Acad. Sci. USA 88:8039-8043; Ferry et al., 1991, Proc. NatI. Acad. Sci. USA 88:8377-8381; Chowdhury et al., 1991, Science 254:1802-1805; van Beusechem. et al., 1992, Proc. Nad. Acad. Sci. USA 89:7640-19; Kay et al., 1992, Human Gene Therapy 3:641-647; Dai et al., 1992, Proc. Natl. Acad. Sci. USA 89:10892-10895; Hwu et al., 1993, J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

The promoter driving dsRNA expression in either a DNA plasmid or viral vector may be a eukaryotic RNA polymerase I (e.g. ribosomal RNA promoter), RNA polymerase II (e.g. CMV early promoter or actin promoter or U1 snRNA promoter) or generally RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g. the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Generally, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single SNCA gene or multiple SNCA genes over a period of a week or more are also contemplated by the invention. Successful introduction of the vectors into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The SNCA specific dsRNA molecules can also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Treatment Methods and Routes of Delivery

The following discussion refers to treatment with a dsRNA, e.g., a dsRNA described in Tables 2, 3 or 4. A composition that includes a dsRNA can be delivered to a subject by a variety of routes. Exemplary routes include intrathecal, parenchymal (e.g., in the brain), intravenous, nasal, and ocular delivery. One route of delivery is directly to the brain. The anti-SNCA agents can be incorporated into pharmaceutical compositions suitable for administration. For example, compositions can include one or more species of a dsRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, intranasal, transdermal), oral or parenteral. Parenteral administration includes intravenous drip, subcutaneous, intraperitoneal or intramuscular injection, or intrathecal or intraventricular administration.

The route of delivery can be dependent on the disorder of the patient. For example, a subject diagnosed with PD can be administered an anti-SNCA dsRNA directly to the brain, e.g., directly to the substantia nigra of the brain (e.g., into the striatal dopamine domains within the substantia nigra). A subject diagnosed with multiple system atrophy can be administered a dsRNA directly into the brain, e.g., into the striatum and substantia nigra regions of the brain, and into the spinal cord. A subject diagnosed with Lewy body dementia can be administered a dsRNA directly into the brain, e.g., directly into the cortex of the brain, and administration can be diffuse. In addition to an agent which inhibits SNCA expression, e.g., an anti-SNCA dsRNA, a patient can be administered a second therapy, e.g., a palliative therapy and/or disease-specific therapy. A palliative therapy can be a dopaminergic therapy, for example, such as methyldopa or coenzyme Q10.

In some embodiments, such as for the treatment of Parkinson's Disease, the secondary therapy can be, for example, symptomatic (e.g., for alleviating symptoms), neuroprotective (e.g., for slowing or halting disease progression), or restorative (e.g., for reversing the disease process). Symptomatic therapies include the drugs carbidopa/levodopa, entacapone, tolcapone, pramipexole, ropinerole, pergolide, bromocriptine, selegeline, amantadine, and several anticholingergic agents. Deep brain stimulation surgery as well as stereotactic brain lesioning may also provide symptomatic relief. Neuroprotective therapies include, for example, carbidopa/levodopa, selegeline, vitamin E, amantadine, pramipexole, ropinerole, coenzyme Q10, and GDNF. Restorative therapies can include, for example, surgical transplantation of stem cells.

An anti-SNCA dsRNA can be delivered to neural cells of the brain. Delivery methods that do not require passage of the composition across the blood-brain barrier can be utilized. For example, a pharmaceutical composition containing a dsRNA can be delivered to the patient by injection directly into the area containing the alpha-synuclein aggregates. For example, the pharmaceutical composition can be delivered by injection directly into the brain. The injection can be by stereotactic injection into a particular region of the brain (e.g., the substantia nigra, cortex, hippocampus, or globus pallidus). The dsRNA can be delivered into multiple regions of the central nervous system (e.g., into multiple regions of the brain, and/or into the spinal cord). The dsRNA can be delivered into diffuse regions of the brain (e.g., diffuse delivery to the cortex of the brain).

In one embodiment, the dsRNA can be delivered by way of a cannula or other delivery device having one end implanted in a tissue, e.g., the brain, e.g., the substantia nigra, cortex, hippocampus, or globus pallidus of the brain. The cannula can be connected to a reservoir of dsRNA. The flow or delivery can be mediated by a pump, e.g., an osmotic pump or minipump. In one embodiment, a pump and reservoir are implanted in an area distant from the tissue, e.g., in the abdomen, and delivery is effected by a conduit leading from the pump or reservoir to the site of release. Devices for delivery to the brain are described, for example, in U.S. Pat. Nos. 6,093,180, and 5,814,014.

A dsRNA can be modified such that it is capable of traversing the blood brain barrier. For example, the dsRNA can be conjugated to a molecule that enables the agent to traverse the barrier. Such modified dsRNAs can be administered by any desired method, such as by intraventricular or intramuscular injection, or by pulmonary delivery, for example.

The anti-SNCA dsRNA can be administered ocularly, such as to treat retinal disorder, e.g., a retinopathy. For example, the pharmaceutical compositions can be applied to the surface of the eye or nearby tissue, e.g., the inside of the eyelid. They can be applied topically, e.g., by spraying, in drops, as an eyewash, or an ointment. Ointments or droppable liquids may be delivered by ocular delivery systems known in the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. The pharmaceutical composition can also be administered to the interior of the eye, and can be introduced by a needle or other delivery device which can introduce it to a selected area or structure. The composition containing the dsRNA can also be applied via an ocular patch.

Administration can be provided by the subject or by another person, e.g., a another caregiver. A caregiver can be any entity involved with providing care to the human: for example, a hospital, hospice, doctor's office, outpatient clinic; a healthcare worker such as a doctor, nurse, or other practitioner; or a spouse or guardian, such as a parent. The medication can be provided in measured doses or in a dispenser which delivers a metered dose.

The subject can be monitored for reactions to the treatment, such as edema or hemorrhaging. For example, the patient can be monitored by MRI, such as daily or weekly following injection, and at periodic time intervals following injection.

The subject can also be monitored for an improvement or stabilization of disease symptoms. Such monitoring can be achieved, for example, by serial clinical assessments (e.g., using the United Parkinson's Disease Rating Scale) or functional neuroimaging. Monitoring can also include serial quantitative measures of striatal dopaminergic function (e.g., fluorodopa and positron emission tomography) comparing treated subjects to normative data collected from untreated subjects. Additional outcome measures can include survival and survival free of palliative therapy and nursing home placement. Statistically significant differences in these measurements and outcomes for treated and untreated subjects is evidence of the efficacy of the treatment.

A pharmaceutical composition containing an anti-SNCA dsRNA can be administered to any patient diagnosed as having or at risk for developing a neurodegenerative disorder, such as a synucleinopathy. In one embodiment, the patient is diagnosed as having a neurodegenerative order, and the patient is otherwise in general good health. For example, the patient is not terminally ill, and the patient is likely to live at least 2, 3, 5, or 10 years or longer following diagnosis. The patient can be treated immediately following diagnosis, or treatment can be delayed until the patient is experiencing more debilitating symptoms, such as motor fluctuations and dyskinesis in PD patients. In another embodiment, the patient has not reached an advanced stage of the disease, e.g., the patient has not reached Hoehn and Yahr stage 5 of PD (Hoehn and Yahr, Neurology 17:427-442, 1967). In another embodiment, the patient is not terminally ill. In general, an anti-SNCA dsRNA can be administered by any suitable method. As used herein, topical delivery can refer to the direct application of a dsRNA to any surface of the body, including the eye, a mucous membrane, surfaces of a body cavity, or to any internal surface. Formulations for topical administration may include transdermal patches, ointments, lotions, creams, gels, drops, sprays, and liquids. Conventional pharmaceutical carriers, aqueous, powder or oily bases, thickeners and the like may be necessary or desirable. Topical administration can also be used as a means to selectively deliver the dsRNA to the epidermis or dermis of a subject, or to specific strata thereof, or to an underlying tissue.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

An anti-SNCA dsRNA can be administered to a subject by pulmonary delivery. Pulmonary delivery compositions can be delivered by inhalation by the patient of a dispersion so that the composition, e.g., the dsRNA, within the dispersion can reach the lung where it can be readily absorbed through the alveolar region directly into blood circulation. Pulmonary delivery can be effective both for systemic delivery and for localized delivery to treat diseases of the lungs. In one embodiment, an anti-SNCA dsRNA administered by pulmonary delivery has been modified such that it is capable of traversing the blood brain barrier.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are also suitable for delivery. One of the benefits of using an atomizer or inhaler is that the potential for contamination is minimized because the devices are self contained. Dry powder dispersion devices, for example, deliver drugs that may be readily formulated as dry powders. An iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

An anti-SNCA dsRNA can be administered by an oral and nasal delivery. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily. In one embodiment, an anti-SNCA dsRNA administered by oral or nasal delivery has been modified to be capable of traversing the blood-brain barrier.

In one embodiment, unit doses or measured doses of a composition that include iRNA are dispensed by an implanted device. The device can include a sensor that monitors a parameter within a subject. For example, the device can include a pump, such as an osmotic pump and, optionally, associated electronics.

A dsRNA can be packaged in a viral natural capsid or in a chemically or enzymatically produced artificial capsid or structure derived therefrom.

Dosage. An anti-SNCA dsRNA, e.g., an anti-SNCA dsRNA described in Tables 2, 3, or 4, can be administered at a unit dose less than about 1.4 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×1016 copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA agent per kg of bodyweight. The unit dose, for example, can be administered by injection (e.g., intravenous or intramuscular, intrathecally, or directly into the brain), an inhaled dose, or a topical application. Typical dosages are less than 2, 1, or 0.1 mg/kg of body weight.

Delivery of a dsRNA directly to an organ (e.g., directly to the brain) can be at a dosage on the order of about 0.00001 mg to about 3 mg per organ, e.g., about 0.0001-0.001 mg per organ, about 0.03-3.0 mg per organ, about 0.1-3.0 mg per eye or about 0.3-3.0 mg per organ.

The dosage can be an amount effective to treat or prevent a disease or disorder, e.g., a disease or disorder associated with synucleinopathies.

In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In one embodiment, the effective dose is administered with other traditional therapeutic modalities. In one embodiment, the subject has PD and the modality is a therapeutic agent other than a dsRNA, e.g., other than a double-stranded dsRNA, or sRNA agent. The therapeutic modality can be, for example, levadopa or depronil.

In one embodiment, a subject is administered an initial dose, and one or more maintenance doses of a dsRNA, e.g., a double-stranded dsRNA, or sRNA agent, (e.g., a precursor, e.g., a larger dsRNA which can be processed into an sRNA agent, or a DNA which encodes a dsRNA, e.g., a double-stranded dsRNA, or sRNA agent, or precursor thereof). The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are typically administered no more than once every 5, 10, or 30 days. Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In one embodiment, the dsRNA pharmaceutical composition includes a plurality of dsRNA species. In another embodiment, the dsRNA species has sequences that are non-overlapping and non-adjacent to another species with respect to a naturally occurring target sequence. In another embodiment, the plurality of dsRNA species is specific for different naturally occurring target genes. In another embodiment, the dsRNA is allele specific.

Following successful treatment, it may be desirable to have the patient undergo maintenance therapy to prevent the recurrence of the disease state, wherein the compound featured in the invention is administered in maintenance doses, ranging from 0.01 μg to 100 g per kg of body weight (see U.S. Pat. No. 6,107,094).

The concentration of the dsRNA composition is an amount sufficient to be effective in treating or preventing a disorder or to regulate a physiological condition in humans. The concentration or amount of dsRNA administered will depend on the parameters determined for the agent and the method of administration, e.g. nasal, buccal, or pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a dsRNA, can include a single treatment or a series of treatments. It will also be appreciated that the effective dosage of a dsRNA used for treatment may increase or decrease over the course of a particular treatment. Changes in dosage may result and become apparent from the results of diagnostic assays as described herein. For example, the subject can be monitored after administering a dsRNA composition. Based on information from the monitoring, an additional amount of the dsRNA composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human gene, e.g., a gene that produces a target RNA, e.g., an SNCA RNA. The transgenic animal can be deficient for the corresponding endogenous RNA. In another embodiment, the composition for testing includes a dsRNA that is complementary, at least in an internal region, to a sequence that is conserved between the target RNA in the animal model and the target RNA in a human.

Kits. In certain other aspects, the invention provides kits that include a suitable container containing a pharmaceutical formulation of a dsRNA, e.g., an sRNA agent (e.g., a precursor, e.g., a larger dsRNA which can be processed into a sRNA agent, or a DNA which encodes a dsRNA, e.g., a double-stranded dsRNA, or sRNA agent, or precursor thereof), a dsRNA described in Tables 2, 3, or 4. In certain embodiments the individual components of the pharmaceutical formulation may be provided in one container. Alternatively, it may be desirable to provide the components of the pharmaceutical formulation separately in two or more containers, e.g., one container for a dsRNA preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The invention is further illustrated by the following examples, which should not be construed as further limiting.

### EXAMPLES

### Example 1

Design of dsRNAs targeting SNCA. Double stranded RNAs having the sequences described in Table 1 were synthesized. FIG. 1A shows the sequence of the full-length SNCA mRNA.

### Example 2

### SNCA dsRNAs Decreased Protein Expression In Vitro

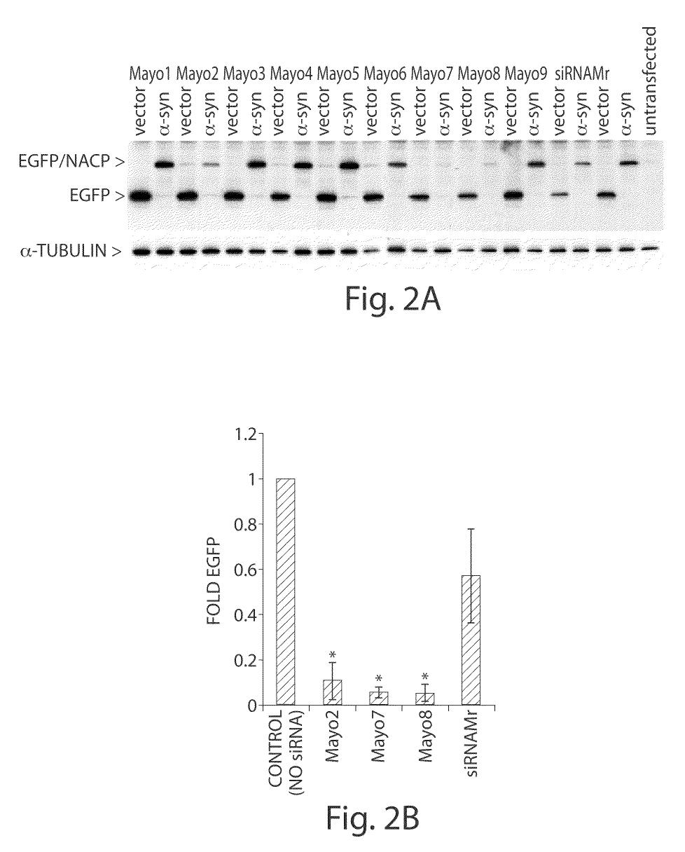

Neuroblastoma cells (BE(2)-M17) were co-transfected with 50 nM dsRNA and a plasmid expressing either EGFP or an alpha-synuclein-EGFP (EGFP/NACP) fusion protein (as used herein NACP is synonymous with the gene product of SNCA). Expression of the EGFP and EGFP/NACP fusion proteins was assayed by Western blot analysis (FIG. 2A).

The in vitro cell-based assay monitors the ability of the test dsRNAs of Table 5 to downregulate expression of an SNCA RNA. The SNCA target RNA in these experiments is fused to an EGFP RNA. Antibodies against EGFP facilitate the detection of an EGFP/NACP fusion protein translated from the RNA.

TABLE 5
dsRNA sequences

| | SEQ | | |
|---|---|---|---|
| | ID | | |
| dsRNA^{a} | NO | Strand | Sequence^{b} |
| SNCA1 | 801 | Sense | 5′-GGUGUGGCAACAGUGGCUGAG-3′ |
| | 802 | Antisense | 3′-UACCACACCGUUGUCACCGACUC-5′ |
| SNCA2 | 803 | Sense | 5′-AACAGUGGCUGAGAAGACCAA-3′ |
| | 804 | Antisense | 3′-CGUUGUCACCGACUCUUCUGGUU-5′ |
| SNCA3 | 805 | Sense | 5′-AUUGCAGCAGCCACUGGCUUU-3′ |
| | 806 | Antisense | 3′-CGUAACGUCGUCGGUGACCGAAA-5′ |
| SNCA4 | 807 | Sense | 5′-AAGUGACAAAUGUUGGAGGAG-3′ |
| | 808 | Antisense | 3′-CGUUCACUGUUUACAACCUCCAC-5′ |
| SNCA5 | 809 | Sense | 5′-GAAGAAGGAGCCCCACAGGAA-3′ |
| | 810 | Antisense | 3′-UACUUCUUCCUCGGGGUGUCCUU-5′ |
| SNCA6 | 811 | Sense | 5′-CGGGUGUGACAGCAGUAGCdTdT-3′ |
| | 812 | Antisense | 3′-dTdTGCCCACACUGUCGUCAUCG-5′ |
| SNCA7 | 813 | Sense | 5′-UCCUGACAAUGAGGCUUAUdTdT-3′ |
| | 814 | Antisense | 3′-dTdTAGGACUGUUACUCCGAAUA-5′ |
| SNCA7s | 815 | Sense | 5′-U*CCUGACAAUGAGGCUUAUdT*dT-3′ |
| | 816 | Antisense | 3′-dT*dTAGGACUGUUACUCCGAAU*A-5′ |
| SNCA8 | 817 | Sense | 5′-CUACGAACCUGAAGCCUAAdTdT-3′ |
| | 818 | Antisense | 3′-dTdTGAUGCUUGGACUUCGGAUU-5′ |
| SNCA8 | 819 | Sense | 5′-C*UACGAACCUGAAGCCUAAdT*dT-3′ |
| s1 | 820 | Antisense | 3′-dT*dTGAUGCUUGGACUUCGGAU*U-5′ |
| SNCA8 | 821 | Sense | 5′-C*UACGAACCUGAAGCCUAAdT*dT-3′ |
| s2 | 822 | Antisense | 3′-dT*dTGAUGCUUGGACUUCGGAU*U-5′ |
| SNCA9 | 823 | Sense | 5′-CUAUUGUAGAGUGGUCUAUdTdT-3′ |
| | 824 | Antisense | 3′-dTdTGAUAACAUCUCACCAGAUA-5′ |
| ALN- | 825 | Sense | 5′-GAACUGUGUGUGAGAGGUCCU-3′-F |
| DP- | 826 | Antisense | 3′-*C*CUUGACACACACUCUCCAGGA-5′ |
| 3000 | | | |
| SiRNA | 827 | Sense | 5′-GACGUAAACGGCCACAAGUUC-3′ |
| Mr | 828 | Antisense | 3′-CGCUGCAUUUGCCGGUGUUCA-5′ |
| SNCA8 | 829 | Sense | 5′-C*UAUGAGCCUGAAGCCUAAdT*dT-3′ |
| s2m | 830 | Antisense | 3′-dT*dTGAUACUCGGACUUCGGAU*U-5′ |

^{a}SNCA name designations are equivalent to Mayo designations (e.g., SNCA1 is equivalent to Mayo1);
^{b}nucleotides marked with * carry a phosphorothioate modification; underlined nucleotides carry a 2′-O-Me modification; “F” indicates a fluorescein conjugate
^{c}nThe target sequences of the dsRNAs are illustrated in FIG. 1A.

Control experiments used in this assay included the use of a dsRNA targeting a RNA (see the lanes marked “siRNA Mr” in FIG. 2A), and cells not transfected with siRNA. Antibodies against alpha tubulin were used as controls to monitor the amount of total protein loaded in each lane. The control experiments indicated that EGFP and the EGFP/NACP proteins are expressed in about equal amounts when in the absence of anti-SNCA dsRNA. The strongest down-regulatory effect of EGFP/NACP expression was observed with Mayo2, Mayo7, and Mayo8 dsRNAs (as used herein, MayoX siRNAs are synonymous with SNCAX dsRNAs). A weaker effect was observed with Mayo1, Mayo6, and Mayo9 dsRNAs. siRNA Mr affected expression of both the vector derived EGFP and the EGFP/NACP conjugate, demonstrating the suitability of the assay.

Densitometry of immunoblots with EGFP and alpha-tubulin antibodies were performed to gain a quantitative measure of protein levels (FIG. 2B). Significant silencing of the EGFP-NACP construct was caused by three of the siRNAs: Mayo2 (89%; p=0.0002), Mayo7 (95%; p=0.0002) and Mayo8 (95%; p=0.0006); Mayo6 was partially active (31%; p=0.21). The EGFP targeted siRNAMr was active against both the vector derived target and the EGFP-NACP target but not to significant levels (e.g. EGFP-NACP silencing was 43%; p=0.21). The SNCA targeted siRNAs had no activity against the vector-derived EGFP. Immunoblots of protein extracts from untransfected cells showed no immunoreactivity to non-specific proteins of the same size as the EGFP and EGFP-NACP targets (27 and 44 kDa respectively).

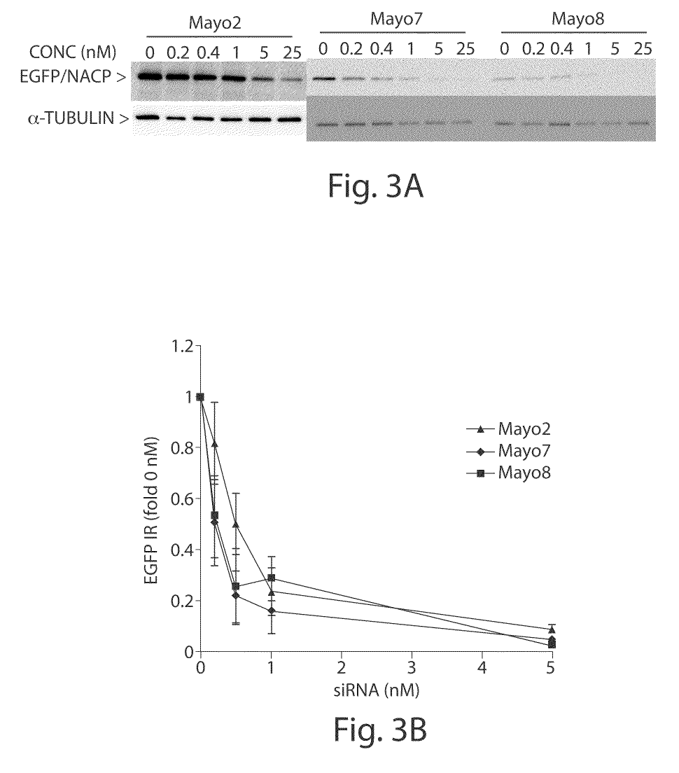

The inhibitory effect of the most effective dsRNAs (Mayo2, Mayo7, and Mayo8) was examined at varying dsRNA concentrations during a 24 h incubation (FIG. 3A). The cells were cotransfected with a vector expressing an EGFP/NACP fusion protein and varying nanmolar concentrations of Mayo2, Mayo7, or Mayo8 siRNA. These siRNAs are equivalent to siRNAs SNCA2, SNCA7, and SNCA8, of Table 5 respectively. The Western blots were stripped of the anti-GFP antibody, and reprobed with anti-tubulin antibody to monitor equivalent loading of protein between samples. The IC₅₀ value was determined from the graphs of densitometry from three independent assays: Mayo2=0.5 nM; Mayo7=0.2 nM; Mayo8=0.2 nM (FIG. 3B).

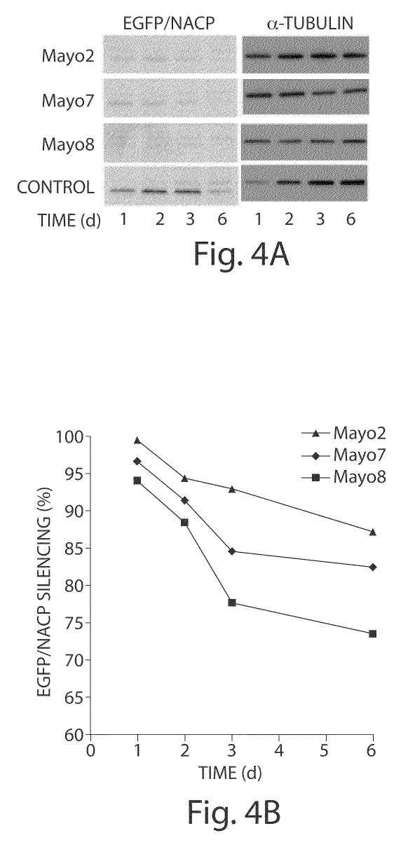

The inhibitory effect of the most effective siRNAs was tested in slowly-dividing neuroblastoma cells in cultures with low levels of serum. BE(2)-M17 cells were transfected with a plasmid expressing the EGFP/NACP fusion protein alone (control) or cotransfected with 50 nM Mayo2, Mayo7, or Mayo8 dsRNAs. Protein samples were harvested at 1, 2, 3 and 6 days post-transfection and assayed by immunoblot (FIGS. 4A and 4B). Fusion protein expression was effectively silenced for at least three days (FIG. 4A). The EGFP-NACP transient expression in the control sample on day 6 was 26% of the day 2 level; this is a reflection of the stability of the plasmid in the cells. Nevertheless, it was possible to measure the silencing up to this point, which steadily decreased: Mayo2-97% to 82%; Mayo7-94% to 73%; Mayo8-99% to 87% (FIG. 4B).

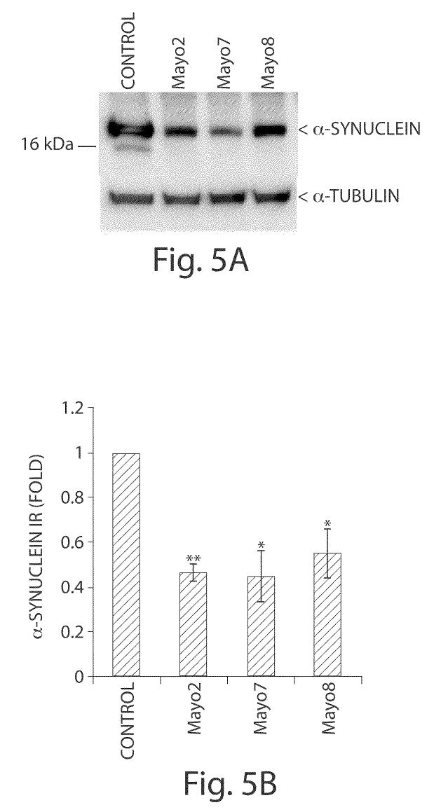

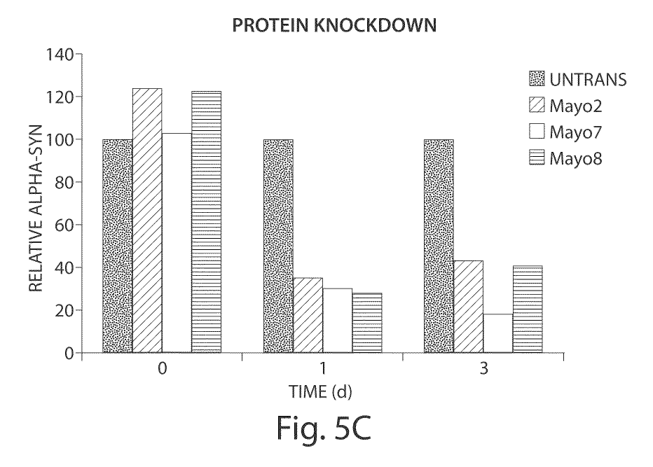

The dsRNAs also inhibited endogenous protein expression in BE(2)-M17 cells. Transfection of 50 nM siRNA (Mayo2, Mayo7, and Mayo8) was performed in the absence of plasmid cotransfection, and the cells were incubated for 24 hours. Endogenous human alpha-synuclein was detected by immunoblot and measured by desitometry following equalization for loading level against alpha-tubulin immunoreactivity. Significant reduction of the alpha-synuclein target was observed with Mayo2 (53%; p=0.0009), Mayo7 (55%; p=0.02) and Mayo8 (45%; p=0.02) (FIG. 5B). A band migrating slightly faster than the main 19 kDa SNCA protein was always present in the control sample and always absent in the siRNA treated samples (FIG. 5A). Only the 19 kDa protein was used for the densitometric analysis. The dsRNAs inhibited endogenous protein expression for at least three days (FIG. 5C).

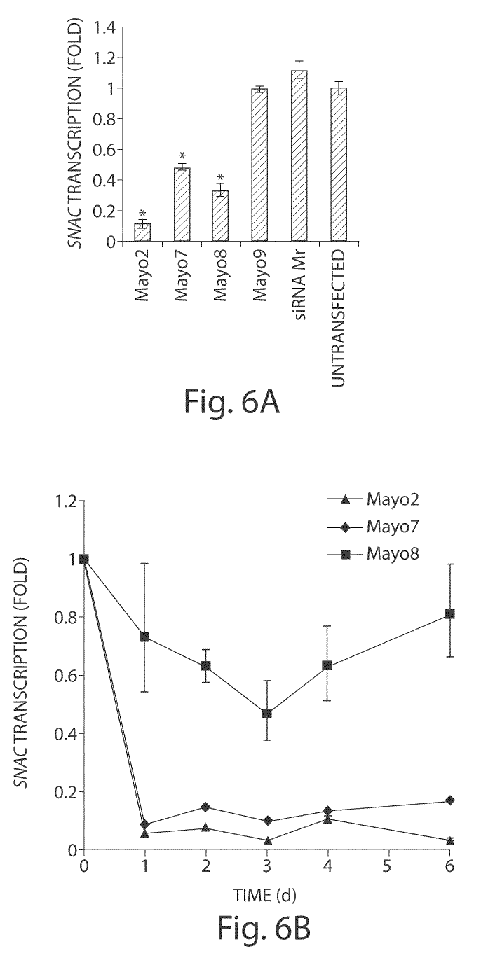

The Mayo2, Mayo7, and Mayo8 siRNAs also inhibited expression of endogenous SNCA RNA in the BE(2)-M17 cells. Cells were treated for 24 h with 50 nM siRNA: siRNAMr, Mayo2, Mayo7, Mayo8 and Mayo9. The latter was included as it targets the 3′-UTR of SNCA, which is not present in the EGFP-NACP conjugate used in the initial screen. An untransfected culture was used as a control. Assays for the human SNCA transcript, equalized against 18S rRNA, were expressed as a proportion of the control. The SNCA transcript was shown to be significantly reduced by Mayo2 (89%; p=0.01), Mayo7 (52%; p=0.04) and Mayo8 (67%; p=0.02), but not by Mayo9 (0.8%; p=0.38) or siRNAMr (−12%; p=0.30) (FIG. 6A). In a time course assay for SNCA transcription after gene silencing, after 6 days, Mayo2 and Mayo8 continued to effectively inhibit endogenous SNCA expression (FIG. 6B). The expression of the target is not transient in this assay, and it was possible to measure stable silencing for Mayo2 (91% to 83%) and Mayo8 (96% to 89%). Silencing by Mayo7 was not sustained well (53% to 19%). Levels of SNCA mRNA in the cells were measured by the Taqman® method of quantitative RT-PCR normalized against 18S rRNA expression levels. Mayo9, which targets the 3′UTR of SNCA, did not inhibit expression of endogenous alpha synuclein RNA.

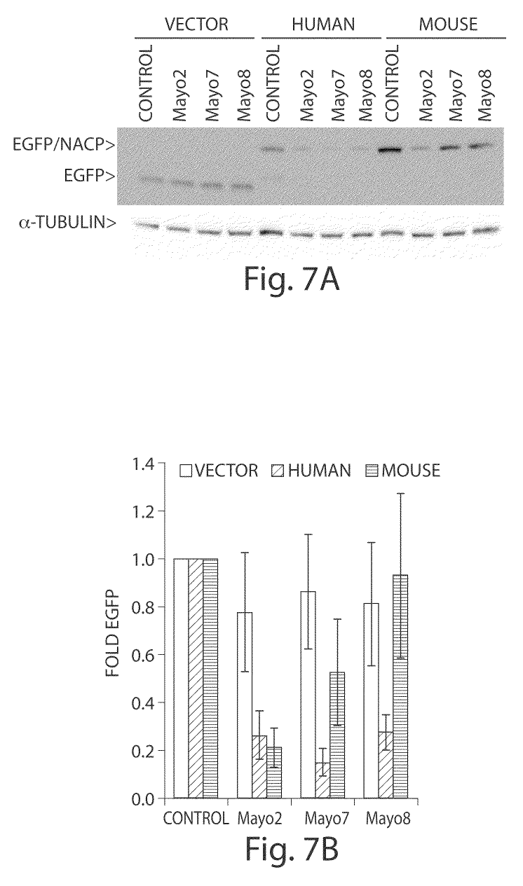

The efficacy of the Mayo2, 7, and 8 dsRNAs were tested against mouse SNCA. BE(2)-M17 cells were cotransfected with a plasmid encoding EGFP (vector) or EGFP conjugated to either human or mouse alpha-synuclein, and Mayo2, Mayo7 or Mayo8. The control was treated with transfection reagent alone. Expression of EGFP and EGFP-NACP was assayed by Western blot (FIG. 7A). While all three dsRNAs inhibited expression of human EGFP-NACP, only Mayo2 inhibited expression in mouse EGFP-NACP. Mayo2 silenced human and mouse SNCA cDNA expression to similar levels (74% and 79% respectively). The mean activities of the other siRNAs were predominantly human SNCA specific: Mayo7-85% human, 47% mouse; Mayo8-73% human, 7% mouse (FIG. 7B). The human and mouse mRNA sequence is identical at the Mayo2 locus, but diverges by two nucleotides at each of the Mayo7 and Mayo8 loci (See Table 6).

TABLE 6

Nucleotide homologies of cDNA at
the loci of the three active SiRNAs^{a}.

SEQ ID NO:

Gene family homology

| | | | |
|---|---|---|---|
| Mayo2 | SNCA | AAcagtggctgaGaaGaccaa | 831 |
| | SNCB | TTcagtggctgaAaaAaccaa | 832 |
| Mayo7 | SNCA | tCCTgacAATGAGGCTTaT | 833 |
| | SNCB | tGAGgacCCACCCCAGGaG | 834 |
| Mayo8 | SNCA | CTACGAACCTGAAGCCTAA | 835 |
| | SNCB | ------------------- | |

Species homology

| | | | |
|---|---|---|---|
| Mayo2 | Human | aacagtggctgagaagaccaa | 836 |
| | Mouse | aacagtggctgagaagaccaa | 837 |
| Mayo7 | Human | tcctgAcaAtgaggcttat | 838 |
| | Mouse | tcctgGcaGtgaggcttat | 839 |
| Mayo8 | Human | ctaCgaAcctgaagcctaa | 840 |
| | Mouse | ctaTgaGcctgaagcctaa | 841 |

^{a}Divergence is highlighted in boldface capitals. Lack of recognizable homology is shown by dashes.

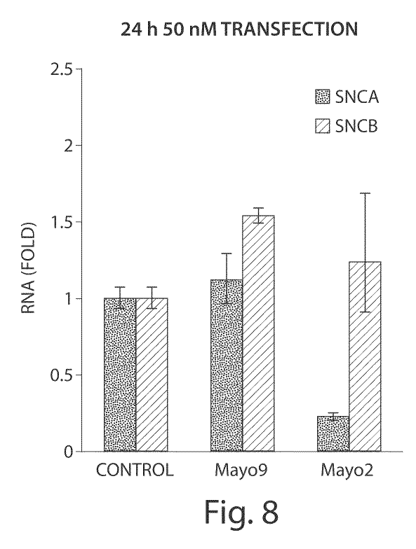

SNCB (beta-synuclein) shares sequence similarity with alpha-synuclein at the Mayo2 locus, but differs in sequence by four nucleotides (Table 6). The efficacy of the Mayo2 was tested against SNCB. BE(2)-M17 cells were transfected with a plasmid expressing the dsRNAs Mayo2 or Mayo9. Expression of endogenous SNCA and SNCB RNA was assayed by Taqman® method quantitative RT-PCR. Mayo2 inhibited expression of SNCA but not expression of SNCB (FIG. 8). As was expected, Mayo9 did not inhibit expression of SNCB or SNCA.

### Example 3

### Stability of SNCA siRNAs

The stability of the sense and antisense strands of the SNCA siRNAs was examined in 90% mouse serum or 90% human serum, and in mouse brain tissue. To perform the stability assays, siRNA was radioactively labeled on the sense or antisense strand (both strands were assayed for stability in the serum and brain tissue). Protein extracts were prepared from mouse brain, and 100 nM siRNA duplex was incubated with the extract at 37° C. At time points over the course of 4-5 hours, sample was removed and analyzed on a polyacrylamide denaturing gel.

The stability of Mayo2, 7, and 8 was tested in mouse serum and brain extract. Further, the cleavage sites of Mayo7 and Mayo8 were mapped by T1 analysis. RNAse T1 cleaves 3′ of G nucleotides, and T1 digestion of an RNA that has a known sequence provides orientation and a basis for comparison to detect non-RNAse T1 cleavage sites. T1 was used to map the cleavage sites of Mayo7 (also called SNCA7, or AL-DUP-1477) and Mayo8 (also called SNCA8, or AL-DUP-1478) siRNAs (See Table 5). Mayo7 and 8 were 5′ end labeled with ³²P on the sense strand, and RNAse T1 digestion was performed for four hours. The samples were analyzed by electrophoresis. Mayo7 was found to be susceptible to endonucleolytic cleavage 3′ of U16 and U17. Mayo8 was found to be susceptible to endonucleolytic cleavage 3′ of U16.

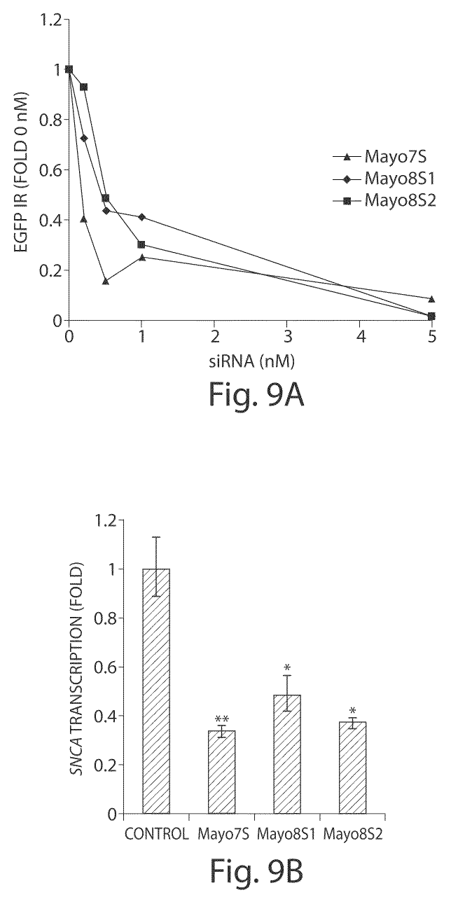

To increase stability of the Mayo7 and Mayo8 siRNAs, nucleotides were modified with a 2′-O-Me group or a phosphorothioate linkage to create Mayo7s, Mayo8s1, and Mayo8s2 (Table 5). The modified siRNAs (50 nM) were cotransfected with an EGFP-NACP vector into cells as described above. Untransfected cells served as a control. A dose response assay of the stabilized siRNAs was performed to ensure that the chemical modifications did not alter their activity. A 24 h co-transfection with pEGFP-NACP was performed using 0, 0.2, 0.4, 1.0, 5.0 and 25.0 nM siRNA. Silencing of SNCA was assayed by immunoblot against EGFP (FIG. 9A). The IC50 of each molecule was interpolated from the graphs of densitometry: Mayo7S=0.2 nM; Mayo8S1=0.4 nM; Mayo8S2=0.5 nM. Further characterization included qRT-PCR assays of RNA prepared from cells transfected with 50 nM stabilized siRNAs for 24 h. Assays for the SNCA transcript were equalized against 18S rRNA as before, and expressed as a proportion of the untreated control (FIG. 9B). The SNCA transcript was shown to be significantly reduced by Mayo7S (66%; p=0.002), Mayo8S1 (52%; p=0.04) and Mayo8S2 (63%; p=0.04).

The modified and unmodified Mayo8 siRNAs were analyzed by Stains-All (cat. #E9379, Sigma, St. Louis, Mo.), which was performed as follows. All solutions were prepared in nuclease-free water (cat. #9930, Ambion, Austin, Tex.), using nuclease-free reagents. A 50 μM stock of dsRNA for use in the stability assays was prepared by mixing 50 μM sense strand RNA and 50 μM antisense strand in 1×PBS. This mixture was incubated at 90° C. for 2 minutes to denature the nucleic acids, then 37° C. for one hour for annealing.

To perform the stability assay, human serum from clotted male whole blood type AB (cat. #H1513, Sigma, St. Louis, Mo.) was used. Serum was thawed on ice, and mixed with dsRNA to a final concentration of about 4.5 μM (i.e., about 4.2 μg, or about 300 pmoles dsRNA). At time point “0,” one control sample was frozen on dry ice immediately following addition of dsRNA to serum, and the sample was stored at −80° C. For other time points (15, 30, 60, 120, and 240 minutes in human serum), the samples were incubated at 37° C. in a Thermomixer (Eppendorf, Hamburg, Germany). At each endpoint, the samples were frozen on dry-ice and stored at −80° C.

To extract the RNA from the serum, samples were thawed on ice, and then 0.5 M NaCl (nuclease free; cat#9760, Ambion, Austin, Tex.) was added to the sample to yield a final concentration of about 0.45 M NaCl. The sample was vortexed briefly (about 5 seconds), and then transferred to a prepared and chilled Phase Lock-Gel-Eppis (Eppendorf, Hamburg, Germany). Five hundred microliters phenol:chloroform:isoamyl alcohol (25:24:1) and 300 μL chloroform were added to the mix. The sample was vortexed briefly for 30 seconds, then centrifuged at 13,200 rpm for 15 minutes at 4° C.

The aqueous phase was transferred to a clean eppendorf tube, and 3M NaOAc, pH 5.2, was added to a final concentration of about 0.1M NaOAc. The solution was vortexed for about 20 seconds and then 1 μL of Glyco Blue (Ambion, Austin, Tex.) was added. The solution was vortexed briefly and gently, then 1 mL ice-cold 100% ethanol was added. The solution was vortexed for about 20 seconds, then stored at −80° C. for one hour, or at −20° C. overnight to precipitate the RNA. Following precipitation, the mixture was centrifuged at 13,200 rpm for 30 min. at 4° C., and the RNA pellet was washed with 500 μL 70% ethanol. The pellet was air-dried, then 30 μL of gel loading buffer (95% formamide, 50 mM EDTA, Xylenecyanol, bromophenol blue) was added to the mix, and the mix vortexed for 2 minutes to resuspend.

The RNA sample was analyzed on a 20 cm×20 cm×0.8 mm (length×width×thickness) 20% polyacrylamide gel. To make the gel, 24 g 8 M Urea, 25 mL 40% (19:1) Acrylamide, and 8 mL formamide was mixed in 1×TBE in a 50 mL solution. Polymerization was activated by 50 uL Temed and 200 uL 10% APS (ammonium persulfate). The gel was run in 1×TBE. The gel was pre-run for 30 minutes at 40 mA. The samples were heated at 100° C. for 5 min. and then immediately chilled on ice. For control experiments, 2 μL of dsRNA was mixed with 8 μL of gel loading buffer. The samples were centrifuged at 13,200 rpm (20 seconds, 4° C.) and 10 μL was loaded onto the gel. The gel was run for about one hour at 40 mA.

To visualize the RNA, the gel was stained with Stains-All solution (cat. #E9379, Sigma, St. Louis, Mo.) (100 mg Stains-All dissolved in 800 mL formamide:water (1:1 v/v)) for 30 minutes. The gel was destained in water for 30-60 minutes as needed. The gel was them imaged on a scanner and analyzed.

For the more heavily modified SNCA8s2, a significant amount of full-length siRNA could be detected following a 24 hour incubation in human serum, whereas no full-length unmodified Mayo8 remained after 30 minutes of serum incubation. Comparison indicated that the unmodified SNCA8 dsRNA is rapidly degraded, the partially modified dsRNA (SNCA8 μl) is partially stabilized, and the further modified (SNCA8s2) is the most stable of the three duplexes.

### Example 4

### Knockdown of Species-Specific SNCA

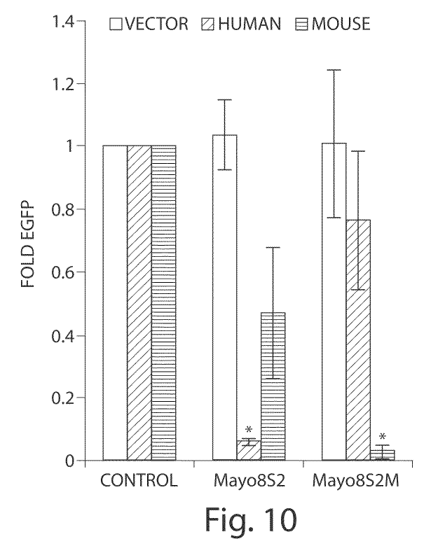

A duplex identical to Mayo8s2 but containing nucleotide modifications to complement the mouse mRNA was constructed to test the activity of these dsRNA in a mouse model system. The murine SNCA siRNA (Mayo8s2M) was compared against the human SNCA siRNA (Mayo8s2) in a co-transfection assay using the pEGFP-Cl (vector), pEGFP-MusNACP (mouse), pEGFP-NACP (human) or plasmid derived targets. Immunoblots of the total protein extracts harvested 24 h post transfection demonstrate that the nucleotide modifications confer species specificity at significant levels: Mayo8s2 silenced human cDNA expression by 94% (p=0.003) and mouse cDNA expression by 53% (p=0.12); Mayo8s2M silenced human cDNA expression by 23% (p=0.24) and mouse cDNA expression by 97% (p=0.007) (FIG. 10).

### Example 5

### Silencing of Endogenous Alpha-Synuclein by Intraparenchymal Infusion of siRNA

The hippocampus and cortex were identified as having the highest expression of SNCA in the murine brain. The following experiments were designed to target SNCA expression in the hippocampus. Using stereotactic surgery, infusion cannulae were implanted into the hippocampus of eight-week old, female B6 mice (coordinates from bregma: x=(−)2.0, y=(−)1.5, z=2.0 calculated from Paxinos and Franklin, The Mouse Brain in Stereotaxic Coordinates). Cannulae were implanted into the right hemisphere of the brain. The cannulae were connected via catheters to osmotic mini-pumps (Alzet model 1002) containing approximately one hundred microliters of 2.1 mM siRNA solution in Phosphate Buffered Saline (PBS). The pumps were implanted subcutaneously. The infusion rate of 0.25 microliters per hour resulted in a dose of approximately 180 micrograms of siRNA per day. Infusion continued for a period of fifteen days. Treatment groups were: PBS (n=10), alpha-synuclein duplex (SNCA siRNA; n=8), cholesterol conjugated alpha-synuclein duplex (SNCA siRNA-chol; n=8), luciferase control duplex (n=8), cholesterol conjugated luciferase control duplex (n=10). The sequences of the duplexes, as well as chemical modifications are shown below in Table 7.

TABLE 7
dsRNA sequences tested in murine brain

| | | |
|---|---|---|
| | | SEQ |
| | | ID |
| siRNA | Sequence | NO: |

| | | | |
|---|---|---|---|
| Luc | S | 5′ cuuAcGcuGAGuAcuucGATsT 3′ | 842 |
| control | AS | 5′ UCGAAGuACUcAGCGuAAGTsT 3′ | 843 |
| Luc | S | 5′ cuuAcGcuGAGuAcuucGATsTs-chol 3′ | 844 |
| control | AS | 5′ UCGAAGuACUcAGCGuAAGTsT 3′ | 845 |
| chol | | | |
| SNCA | S | 5′ CsuAUGAGCCUGAAGCcuaATsT 3′ | 846 |
| | AS | 5′ usuAGGCUUCAGGCUCAuAGTsT 3′ | 847 |
| SNCA | S | 5′ CsuAUGAGCCUGAAGCcuaATsT-chol 3′ | 848 |
| chol | AS | 5′ usuAGGCUUCAGGCUCAuAGTsT 3′ | 849 |

Key
A, C, G, U-ribonucleotides
c, u-2′-OMe nucleotides
s-phosphorothioate linkage
T-thymidine

Following the infusion period, brains were collected and the regions corresponding to the hippocampus were dissected from each hemisphere. Total RNA was isolated and used to prepare cDNA by random hexamer priming. Relative levels of alpha-synuclein were measured by TaqMan® quantitative PCR using gene expression MGB probes (SNCA Mm0044733_ml, GAPDH Mm99999915_gl, HPRT Mm00446968_ml, Tau Mm00521988_ml; Applied Biosystems). For more accurate normalization among tissues, levels of GAPDH, HPRT and tau were measured and used to determine a normalization factor. Relative levels of alpha-synuclein were calculated for the right and left hemispheres from each animal, and group means and standard deviations were calculated.

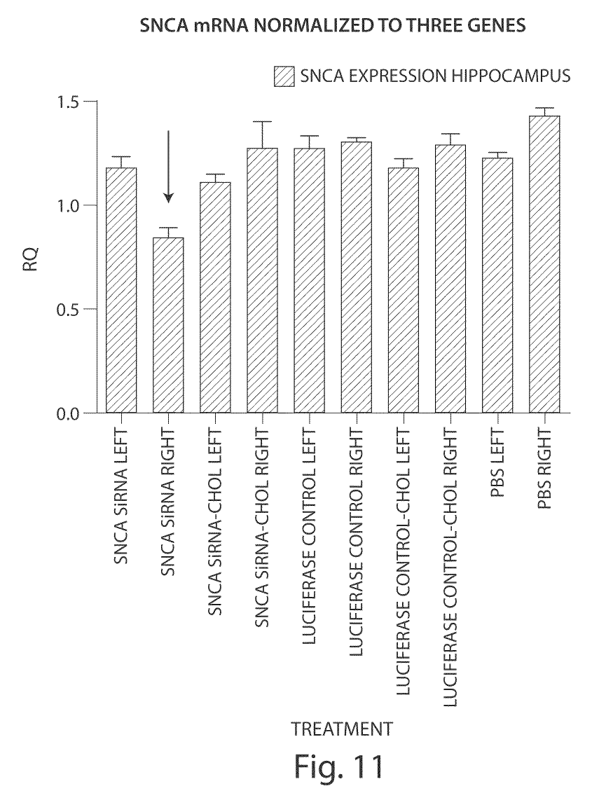

A decrease of alpha-synuclein expression of approximately 30% (right vs left side) was measured in the animals infused with the SNCA siRNA. Statistical significance (p=0.036) was determined by T-test (FIG. 11).

In other experiments, SNCA siRNA (siRNA), luciferase siRNA (luc), SNCA siRNA conjugated to cholesterol (siRNA-c), luciferase siRNA conjugated to cholesterol (luc-c), or PBS was infused into the right CA1 of the hippocampus of wildtype C57BL6 female mice. Continuous infusion of the siRNA or PBS solution was performed for a period of 15 days with Alzet mini pumps connected to cannulae which were surgically implanted into the right CA1. The left CA1 was injected and utilized for an additional control.

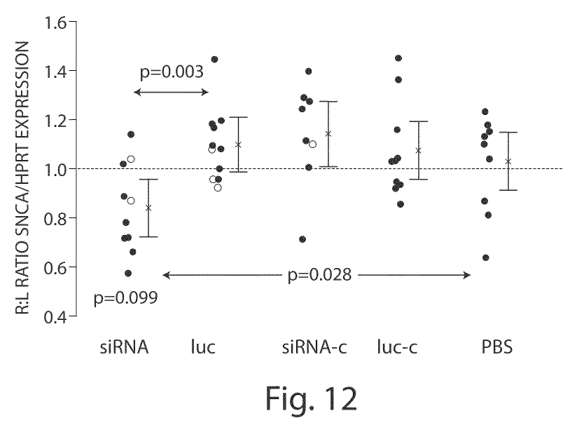

Hippocampal infusion of the Mayo8s2M RNA resulted in significant knockdown of SNCA RNA when assessed by Taqman® quantitative real-time PCR. Normalization was performed against HPRT and GAPDH as endogenous controls. Quantitative RT-PCR analysis demonstrated that SNCA expression was significantly decreased in the right (treated) hippocampus of animals which had received SNCA siRNA compared to luciferase siRNA (p=0.003) or PBS (p=0.028) (FIG. 12). Given the considerable impact that the SNCA-specific siRNA had on SNCA expression when compared to luciferase and PBS treated control mice, SNCA-siRNA treated versus the untreated contralateral sides showed only an 18% knockdown of SNCA expression (p=0.1) (FIG. 12). This effect may have been due to partial diffusion of the SNCA-siRNA into the contralateral hippocampus.

### Example 6

### In Situ Hybridization Showing Silencing of Endogenous Alpha-Synuclein by Intraparenchymal Infusion of siRNA

Infusion cannulae were implanted into the hippocampus of eight-week old, female B6 mice (coordinates from bregma: x=(−)2.0, y=(−)1.5, z=2.0 calculated from Paxinos and Franklin, The Mouse Brain in Stereotaxic Coordinates). The cannulae were connected via catheters to osmotic mini-pumps (Alzet model 1002) containing approximately one hundred microliters of 2.1 mM siRNA solution in Phosphate Buffered Saline (PBS). The pumps were implanted subcutaneously. The infusion rate of 0.25 microliters per hour resulted in a dose of approximately 180 micrograms of siRNA per day. Infusion continued for a period of fifteen days. Treatment groups were: PBS (n=10), alpha-synuclein duplex (SNCA siRNA; n=9), luciferase control duplex (n=10). The sequences of the duplexes, as well as chemical modifications are shown below (Table 8).

TABLE 8
dsRNA tested in mouse hippocampus.

| | | |
|---|---|---|
| | | SEQ ID |
| siRNA | Sequence | NO: |

| | | | |
|---|---|---|---|
| Luc control | S | 5′ cuuAcGcuGAGuAcuucGATsT 3′ | 850 |
| | AS | 5′ UCGAAGuACUcAGCGuAAGTsT 3′ | 851 |
| SNCA | S | 5′ CsuAUGAGCCUGAAGCcuaATsT 3′ | 852 |
| | AS | 5′ usuAGGCUUCAGGCUCAuAGTsT 3′ | 853 |

Key
A, C, G, U-ribonucleotides
c, u-2′-OMe nucleotides
s-phosphorothioate linkage
T-thymidine

Following the infusion period, brains were dissected rapidly. To ensure sampling consistency, the brain was placed in a tissue matrix and the region anterior and posterior to the hippocampus was removed using a flat blade. The resulting three brain segments were snap frozen on dry ice and stored at −80° C. until use. Frozen sections were cut at 15 μm on a cryostat at −18° C. throughout the entire hippocampus and air dried for 20 minutes before freezing at −80° C. On the day of the experiment, frozen sections were removed on dry ice and dried quickly on a slide warmer at 55° C., then fixed in 4% paraformaldehyde in 0.1M Sorensen's Phosphate buffer for 20 minutes, washed twice in PBS and then dehydrated in ascending alcohols. Hybridization was then performed at 37° C. overnight, in a moist chamber, with approximately 0.02 ng of [α-³³P] dATP 3′ end labeled probe per 1 μl of hybridization buffer (4×SSC, 1×Denhardt's solution, 50% (w/v) de-ionised formamide, 10% (w/v) dextran sulphate, 200 mg/μl herring sperm DNA). The probe (5′GGTCTTCTCAGCCACTGTTGTCACTCCATGAACCAC′3) was designed to exon 3 on mouse SNCA. Specific activity of the probe was >1×10⁸ cpm/μg and after dilution in hybridization buffer corresponded to ˜1×10⁴ cpm/μl. Control hybridizations were also set up that contained a 50-fold molar excess of unlabelled probe to determine non-specific signal. Slides were washed in 1×SSC at room temperature (RT) to remove excess hybridization buffer; three times, each for 30 minutes, at 55° and at RT for 60 minutes. Slides were then dipped for 30 seconds in 70% (v/v) ethanol/300 mM ammonium acetate, then for 30 seconds in absolute alcohol, air dried and co-exposed with ¹⁴C microscale standards (Amersham) to Biomax MS film (Kodak) for 7-10 days.

The Metamorph software (Universal imaging) was used to perform densitometry. Specifically, optical density of mRNA labeled with the SNCA specific probe was measured in a standard square with and area of 240 pixels² in the cortex. Optical density was measured and values were compared to optical density of the known ¹⁴C standards. From these values and a graph was constructed and concentration of radioactivity in nCi/g in each sample was extrapolated. A t-test was used to determine if there was difference between groups.

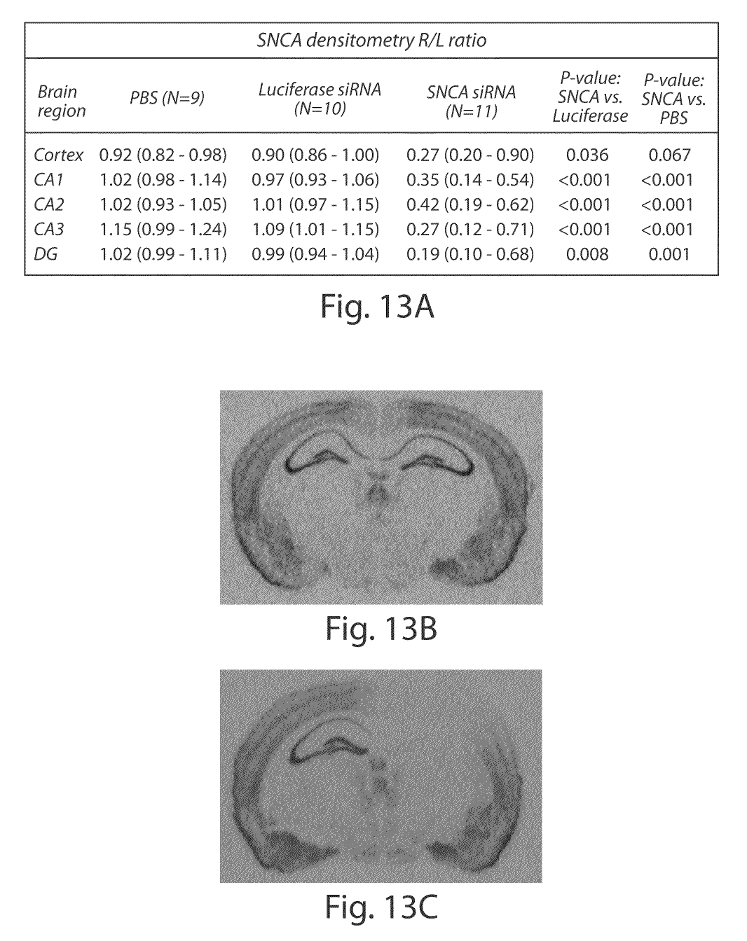

The right CA1 was infused with PBS, siRNA to luciferase, or siRNA against a SNCA target. Densitometry was used to determine efficacy of SNCA siRNA, and SNCA expression in the right side was compared to the uninjected left side and compared across treatment groups. Ratios were calculated for each animal between the injected side and the uninjected side (FIG. 13A). The least reduction in SNCA levels, shown as a reduction in R:L (Right:Left) ratio, was observed in the cortex (−71% between PBS and SNCA siRNA treated animals, p=0.067), likely reflecting the fact that the cortex spans from regions adjacent to the infusion site to regions quite distant from the infusion site and thus less likely to be affected by the siRNA. Significant reductions in SNCA levels were observed in the CA1 (−66%, p<0.001), CA2 (−59%, p<0.001), CA3 (−77%, p<0.001), and dentate gyrus (−81%, p=0.001) when SNCA siRNA treated animals were compared to PBS treated animals (FIGS. 13B and 13C). Similar results were obtained when SNCA siRNA treated animals were compared to luc siRNA treated control mice. Reduction in SNCA levels was confirmed by immunostaining for murine α-synuclein protein. Notably, alpha-synuclein levels in the cell bodies of the hippocampus were decreased, while α-synuclein in projections from distal regions persisted. Decreased SNCA levels in the mice treated with SNCA siRNA was not due to neuronal loss, as assessed by Toluidine Blue staining of in situ sections. Additionally, activation of microglia was not observed with Iba1 immunostaining, suggesting that SNCA siRNA infusion is not associated with gross toxicity or nonspecific inflammatory changes.

### Example 7

### Resilience of SNCA Knockdown in Mice

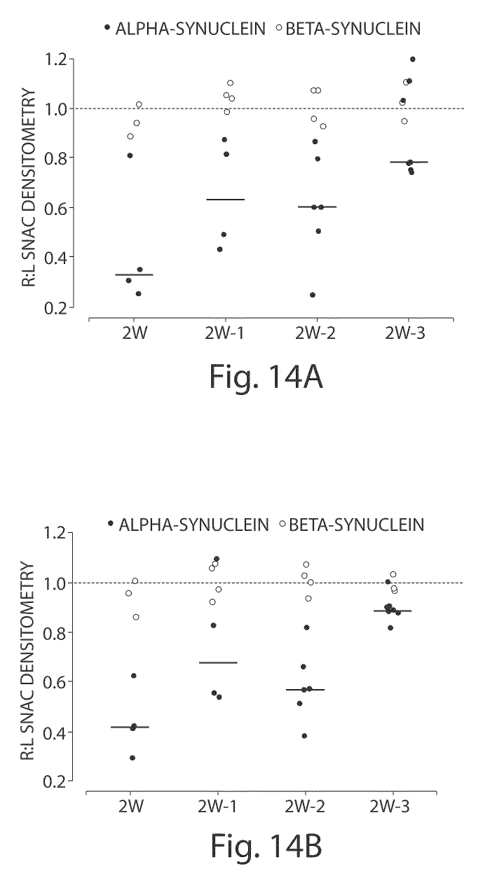

SNCA siRNA was infused into the right CA1 of four cohorts in order to determine the length of time SNCA expression can be repressed following siRNA treatment. Following 15 days infusion, the first cohort (2 W) was harvested as above, while the cannulaes were removed from the remaining cohorts which were then allowed to age for 1 week (2 W-1 W), 2 weeks (2 W-2 W), or three weeks (2 W-3 W) post-infusion. Following in situ for SNCA (FIGS. 14A and 14B), we observed approximately 50% knockdown in SNCA expression in the right cortex, CA1, CA2, CA3, and dentate gyrus which replicated our previous experiments. SNCA levels remained qualitatively reduced 1 week post-infusion in the dentate gyrus and 2 weeks post-infusion in the CA1, CA2, CA3, and cortex. By three weeks post-infusion, SNCA levels in the cortex, CA2, CA3, or dentate gyrus of the siRNA infused side neared control levels. SNCA levels in the right CA1, the site of injection, remained noticeably reduced when compared to the uninjected control side through three weeks post-infusion. As in the earlier studies, we saw no impact of SNCA siRNA on the levels of SNCB at any timepoint (FIGS. 14A and 14B).

### Example 8

### Method of Treating a Patient Diagnosed with a Synucleinopathy

A patient diagnosed with a synucleinopathy can be administered a pharmaceutical composition containing a dsRNA that targets the SNCA gene. The composition can be delivered directly to the brain by a device that includes an osmotic pump and mini-cannula and is bilaterally implanted into the patient.

Prior to implantation of the device, the patient receives an MRI with stereotactic frame. A computer-guided trajectory is used for delivery of the cannula to the brain. The mini-pump device is implanted into the abdomen, and then the patient is hospitalized for 2-3 days to monitor for hemorrhaging.

Approximately two weeks post-implantation of the pump, the patient can receive an MRI to check the implanted device. If the human is healing well, and no complications have occurred as a result of implanting the device, then the anti-SNCA composition can be infused into the pump, and into the cannula. A test dose of the anti-SNCA agent can be administered prior to the initiation of the therapeutic regimen.

MRIs taken at 3 months, six months, and one year following the initial treatment can be used to monitor the condition of the device, and the reaction of the patient to the device and treatment with the dsRNA. Clinicians should watch for the development of edema and an inflammatory response. Following the one-year anniversary of the initiation of the treatment, MRIs can be performed as needed.

The patient can be monitored for an improvement or stabilization in disease symptoms throughout the course of the therapy. Monitoring can include serial clinical assessments and functional neuroimaging, e.g., by MRI.

### Other Embodiments

A number of embodiments featured in the invention have been described. Nevertheless, it will be understood that various modifications may be made without departing from the spirit and scope of the invention. Accordingly, other embodiments are within the scope of the following claims.

What is claimed is:

1. A double-stranded ribonucleic acid (dsRNA), wherein said dsRNA comprises at least two sequences that are substantially complementary to each other and wherein a sense strand of the dsRNA comprises a first sequence and an antisense strand of the dsRNA comprises a second sequence comprising a region that is substantially complementary to the corresponding region of an mRNA encoding SNCA, wherein said region is less than 30 nucleotides in length, and wherein said first sequence is selected from the group consisting of said sense strand sequences in Tables 2, 3, and 4, and wherein said second sequence is selected from the group consisting of said antisense strand sequences in Tables 2, 3, and 4.

2. The dsRNA of claim 1, wherein the dsRNA comprises a dublex region 18-25 nucleotides in length.

3. The dsRNA of claim 1, wherein the dsRNA comprises a nucleotide overhang having 1 to 4 nucleotides.

4. The dsRNA of claim 1, wherein said dsRNA comprises at least one modified nucleotide.

5. The dsRNA of claim 4, wherein said modified nucleotide is chosen from the group consisting of: a 2′-O-methyl modified nucleotide, a nucleotide comprising a 5′-phosphorothioate group, and a terminal nucleotide linked to a cholesteryl derivative or dodecanoic acid bisdecylamide group.

6. The dsRNA of claim 4, wherein said modified nucleotide is chosen from the group consisting of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

7. A cell comprising the dsRNA of claim 1.

8. A pharmaceutical composition, comprising a dsRNA of claim 1 and a pharmaceutically acceptable carrier.

9. A method for inhibiting the expression of an alpha-synuclein gene in a cell, the method comprising:
(a) introducing into the cell a double-stranded ribonucleic acid (dsRNA) of claim 1; and
(b) maintaining the cell produced in step (a) for a time sufficient to obtain degradation of an mRNA transcript of the alpha-synuclein gene, thereby inhibiting expression of the alpha-synuclein gene in the cell.

10. A method of treating, preventing or managing a neurodegenerative disorder comprising administering to a patient in need of such treatment, prevention or management a therapeutically or prophylactically effective amount of a dsRNA of claim 1.

11. The method of claim 10, wherein the neurodegenerative disorder is a synucleinopathy.

12. The method of claim 10, wherein the neurodegenerative disorder is Parkinson's disease.

13. The method of claim 10, wherein the neurodegenerative disorder is Alzheimer's disease, multiple system atrophy, or Lewy body dementia.

14. A method of treating a human comprising:
identifying a human diagnosed as having or at risk for developing a neurodegenerative disorder, and
administering a dsRNA of claim 1.

15. The method of claim 14, wherein the dsRNA comprises a modification that causes the dsRNA to have increased stability in a biological sample.

16. The method of claim 14, wherein the dsRNA comprises a phosphorothioate or a 2′-OMe modification.

17. The method of claim 14, wherein the neurodegenerative disorder is a synucleinopathy.

18. The method of claim 14, wherein the neurodegenerative disorder is Parkinson's disease.

19. The method of claim 14, wherein the neurodegenerative disorder is Alzheimer's disease, multiple system atrophy, or Lewy body dementia.

20. The method of claim 14, wherein the duplex region of the dsRNA is 18-25 nucleotides in length.

21. The method of claim 14, wherein the dsRNA comprises a nucleotide overhang having 1 to 4 unpaired nucleotides.

22. A vector for inhibiting the expression of an alpha-synuclein gene in a cell, said vector comprising a regulatory sequence operably linked to a nucleotide sequence that encodes at least one strand of a dsRNA of claim 1.

23. A cell comprising the vector of claim 22.
